(19) United States
(12) Patent Application Publication
(10) Pub. No.: US 2018/0042873 A1
(43) Pub. Date: Feb. 15, 2018

(54) NOVEL METHODS

(71) Applicant: Aeromics, Inc., Cleveland, OH (US)

(72) Inventors: Marc F. PELLETIER, Shaker Heights, OH (US); George William FARR, Rocky River, OH (US); Paul Robert MCGUIRK, Spring Hill, FL (US)

(73) Assignee: Aeromics, Inc., Cleveland, OH (US)

(21) Appl. No.: 15/526,706

(22) PCT Filed: Nov. 13, 2015
(86) PCT No.: PCT/US2015/060731

§ 371 Date: May 12, 2017

Related U.S. Application Data
(60) Provisional application No. 62/080,241, filed on Nov. 14, 2014.
(60) Provisional application No. 62/079,541, filed on Nov. 13, 2014.

(51) Int. Cl. A61K 31/167; A61K 31/661; A61K 31/6615; A61K 45/06; C07C 235/64; C07F 9/145; A61K 9/08; A61K 9/00; A61K 31/222; A01N 1/02

(52) U.S. Cl. CPC A61K 31/167; A61K 31/222; A61K 31/661; A61K 31/6615; A01N 1/0226; C07C 235/64; C07F 9/145; A61K 9/08; A61K 9/0053; A61K 9/0019; A61K 45/06

(57) ABSTRACT

Provided are uses of selective aquaporin inhibitors, e.g., of aquaporin-4 or aquaporin-2, e.g., certain phenylbenzamide compounds, for the treatment or prophylaxis of transplant rejection and the protection of the heart during heart surgery. Provided is the use of selective aquaporin inhibitors, e.g., of aquaporin-4 (AQP4) or aquaporin-2 (AQP2) for the treatment or prophylaxis of transplant rejection and for the protection of the heart during heart surgery.

This application claims priority to and the benefit of U.S. Provisional Applications No. 62/079,541, filed Nov. 13, 2014, and 62/080,241, filed Nov. 14, 2014, the contents of each of which are incorporated herein by reference in their entireties.

### FIELD

Provided are uses of selective aquaporin inhibitors, e.g., of aquaporin-4 or aquaporin-2, e.g., certain phenylbenzamide compounds, for the treatment or prophylaxis of transplant rejection.

### BACKGROUND

Aquaporins are cell membrane proteins that act as molecular water channels to mediate the flow of water in and out of the cells. While there is some degree of passive diffusion or osmosis of water across cell membranes, the rapid and selective transport of water in and out of cells involves aquaporins. These water channels selectively conduct water molecules in and out of the cell, while blocking the passage of ions and other solutes, thereby preserving the membrane potential of the cell. Aquaporins are found in virtually all life forms, from bacteria to plants to animals. In humans, they are found in cells throughout the body.

Transplantation is the transfer (engraftment) of cells, tissues, or organs from a donor to a recipient. Transplant recipients face a lifetime of immunosuppressive therapy and the risk of losing the new organ due to rejection. In addition, immunosuppressants suppress all immune responses and contribute to post-transplantation complications, including death due to overwhelming infection. Although improvements have been made in the transplantation process, rejection remains a common complication following transplantation. Transplant rejection occurs when the immune system of the transplant recipient attacks the transplanted organ or tissue.

Organ preservation, for example during storage and transport, is a major determinant of graft outcome after revascularization. Organ transplants have a higher frequency of success when performed immediately after excision from the donor.

There remains a need for methods to promote cell, tissue, and organ transplantation tolerance in patients. In addition, there remains a need for methods of tissue and organ preservation.

### BRIEF SUMMARY

Provided is the use of selective aquaporin inhibitors, e.g., of aquaporin-4 (AQP4) or aquaporin-2 (AQP2) for the treatment or prophylaxis of transplant rejection and for the protection of the heart during heart surgery.

Further provided are methods for the treatment or prophylaxis of transplant rejection, and for the protection of the heart during heart surgery, comprising administering to a patient in need thereof an effective amount of an aquaporin inhibitor, e.g., an inhibitor of AQP2 or AQP4, for example a phenylbenzamide, e.g., a compound of Formula I:

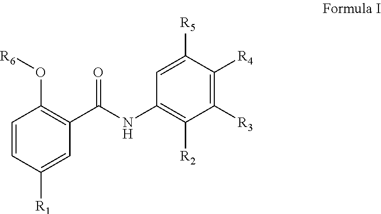

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof.

Further provided is the use of selective aquaporin inhibitors, e.g., of aquaporin-4 or aquaporin-2, for cell, tissue, or organ preservation.

Further provided are methods of cell, tissue, or organ preservation comprising contacting the cell, tissue, or organ with an aquaporin inhibitor, e.g., an inhibitor of AQP2 or AQP4, for example a phenylbenzamide, e.g., a compound of Formula I:

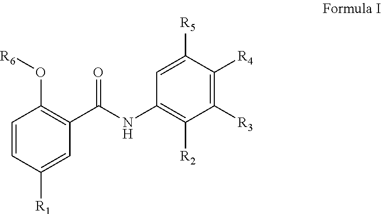

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

FIG. 1 depicts results of a cardiac allograft rejection model.

FIG. 2 shows apoptotic cells in hearts maintained in Ringer's solution for 8 hours in cold storage compared to apoptotic cells in hearts maintained in an APQ4-inhibitor supplemented Ringer's solution.

FIG. 3 shows a heart maintained in un-supplemented Ringer's solution compared to a heart maintained in supplemented Ringer's solution.

### DETAILED DESCRIPTION

The following description of the preferred embodiments is merely exemplary in nature and is in no way intended to limit the invention, its application, or uses.

Aquaporin-4 (AQP4) is reportedly found in mammalian hearts at both the mRNA and protein level. Aquaporin-2 (AQP2) is the primary route of water movement at the collecting duct in the kidney. Several AQPs are reportedly expressed in the lung and airways: AQP1 in microvascular endothelia, AQP3 and AQP4 in airway epithelia, and AQP5 in type I alveolar epithelial cells, submucosal gland acini, and a subset of airway epithelial cells. AQP0, AQP1, AQP4, AQP8, and AQP9 are reportedly expressed in liver cells at both the mRNA and protein level.

Certain aquaporin inhibitors are described in International Application No. PCT/US2013/040194, which is incorporated herein by reference in entirety.

Damage to organs during transplantation occurs in two phases.

The first, the warm ischemic phase, includes the time from the interruption of circulation to the donor organ to the time the organ is flushed with hypothermic preservation solution. The second, the cold ischemic phase, occurs when the organ is preserved in a hypothermic state prior to transplantation into the recipient.

The stability of the cell membrane to chemical and water permeability depends on the integrity of the lipid bilayer and on control of temperature, pH, and osmolarity. Organ ischemia and preservation may disrupt these relations. Lowering the temperature may cause changes in membrane stability and may alter the function of membrane-bound enzymes. Hypothermia-induced structural changes in the membrane may increase permeability, which contributes to cell swelling. Hypertonic organ-preservation solutions may minimize those alterations.

The sodium-potassium adenosine triphosphatase (Na—K ATPase) maintains the ionic composition of the cell. The pump may be disrupted because of the lack of adenosine triphosphate (ATP) production and by excessive production of hydrogen ions because of anaerobic metabolism during ischemia. When the sodium-potassium ATPase pump is paralyzed, potassium moves out of the cell and diffuses down its concentration gradient to the extracellular space, whereas sodium, which is normally kept at a low concentration in the cell, enters. This ionic shift may cause cell swelling and disruption of the cell if unchecked. Preservation solutions with electrolyte compositions similar to the milieu inside the cell may minimize the osmotic gradients.

Transplants may be the patient's own tissue (autografts, e.g., bone, bone marrow, and skin grafts), genetically identical (syngeneic [between monozygotic twins]) donor tissue (isografts), genetically dissimilar donor tissue (allografts or homografts), or grafts from different species (xenografts or heterografts). Transplanted tissue may be cells (as for hematopoietic stem cell [HSC], lymphocyte, and pancreatic islet cell transplants), parts or segments of an organ (as for hepatic or pulmonary lobar transplants and skin grafts), or entire organs (as for heart or kidney transplants).

Allograft transplant recipients are at risk of graft rejection; the recipient's immune system recognizes the graft as foreign and seeks to destroy it. Rejection may be hyperacute, accelerated, acute, and/or chronic.

Hyperacute rejection includes rejection that occurs within 48 hours of transplantation and may be caused by preexisting complement-fixing antibodies to graft antigens (presensitization), for example in the case of xenografts. Hyperacute rejection may be characterized by small-vessel thrombosis and graft infarction.

Accelerated rejection includes rejection that occurs 3 to 5 days after transplantation and is caused by preexisting noncomplement-fixing antibodies to graft antigens. Accelerated rejection may be characterized histopathologically by cellular infiltrate with or without vascular changes.

Acute rejection includes graft destruction after transplantation, which may be caused by a T cell-mediated delayed hypersensitivity reaction to allograft histocompatibility antigens. Acute rejection may be mediated by a de novo anti-graft T-cell response, not by preexisting antibodies. Acute rejection may occur about 5 days after transplantation. Acute rejection may be characterized by mononuclear cellular infiltration, with varying degrees of hemorrhage, edema, and necrosis. Vascular integrity may be maintained, although vascular endothelium may be a primary target.

Chronic rejection includes graft dysfunction, often without fever, typically occurring months to years after transplantation but sometimes within weeks. There may be multiple causes, including early antibody-mediated rejection, periprocedural ischemia and reperfusion injury, drug toxicity, infection, and vascular factors (e.g., hypertension, hyperlipidemia). Proliferation of neointima consisting of smooth muscle cells and extracellular matrix (transplantation atherosclerosis) may gradually and eventually occlude vessel lumina, resulting in patchy ischemia and fibrosis of the graft.

Without intending to be bound by theory, it is hypothesized that if damage to the transplanted organ can be minimized at the time of transplant, this reduces the risk of subsequent rejection, including acute and/or chronic rejection. Thus, prophylaxis of acute or chronic rejection would include actions taken around the time of the transplant, as well as administration of immunosuppressive and/or anti-inflammatory agents administered later for specific control of lymphocyte response.

Heart surgery, e.g., open heart surgery, is another situation wherein a tissue is subjected to the risk of hypoxia and ischemia. The typical open heart surgical procedure involves a prolonged stoppage of the heart and the connection of the patient to a heart-lung machine to provide artificial pumping and gas exchange for the blood. In addition, the surgical procedure itself may also temporarily disrupt the flow of blood in the small vessels that supply the cardiac muscle (e.g., the coronary arteries) with oxygen. As a result, the cardiac muscle is susceptible to hypoxic damage during open heart surgery. Such damage may affect the patient's chances for full recovery. Without being bound by theory, it is hypothesized that minimization of ischemic damage during open heart surgery will improve patient outcomes and survival. Thus, the use of an aquaporin inhibitor, for example a phenylbenzamide of Formula I, is believed to improve the survival of patients undergoing heart surgery.

Edema is the accumulation of excess fluid in a fluid compartment. The accumulation may occur in cells (cellular edema), intercellular spaces within tissues (interstitial edema), or in potential spaces within the body. Cellular edema may be caused by the entry of water into the cells, causing them to swell. It may occur because of decreased osmolality of the fluid surrounding the cells, as in hypotonic fluid overload, or increased osmolality of the intracellular fluid, as in conditions that decrease the activity of the sodium pump of the cell membrane, allowing the concentration of sodium ions within the cell to increase. In particular embodiments of the methods disclosed herein, edema refers to cellular edema.

In particular embodiments of the methods disclosed herein, “amount effective to inhibit an aquaporin” or “amount effective to inhibit the aquaporin” is not an amount that has inhibitory action against NF-κB activation.

As used herein, “concurrently” means the agents are administered simultaneously or within the same composition. In some embodiments, the compounds are administered simultaneously. In some embodiments, the compounds are administered within the same composition.

In one embodiment, provided is a method (Method 1) for treatment or prophylaxis of transplant rejection, inhibiting rejection of transplanted biological material, or prophylaxis, treatment, or control of edema consequent to a transplant, comprising administering to a patient in need thereof, before and/or after the transplant, an effective amount of a phenylbenzamide, e.g., an effective amount of a compound of Formula I:

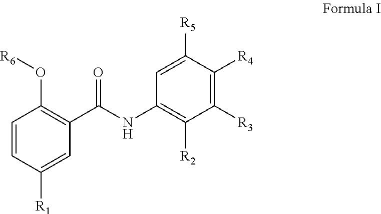

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof,
for example,
1.1. Method 1 comprising treatment or prophylaxis of transplant rejection.
1.2. Method 1 comprising inhibiting rejection of transplanted biological material.
1.3. Method 1 comprising prophylaxis, treatment, or control of edema consequent to a transplant.
1.4. Any of Method 1 et seq. wherein the phenylbenzamide is as described in U.S. Patent Publication No. 2010/0274051, which is incorporated herein by reference in entirety.
1.5. Any of Method 1 et seq. wherein the phenylbenzamide is as described in U.S. Pat. Nos. 7,626,042 or 7,700,655, both of which are incorporated herein by reference in entireties.
1.6. Any of Method 1 or 1.1-1.3 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
1.7. Method 1.6 wherein R₁ is selected from chloro and bromo; R₃ and R₅ are both trifluoromethyl; and R₂, R₄ and R₆ are all H, e.g., wherein the compound of Formula I is selected from:

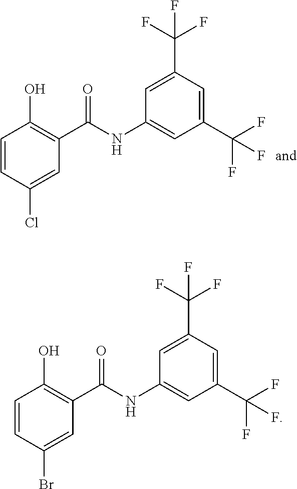 [SMILES: O=C(Nc1cc(C(F)(F)F)cc(C(F)(F)F)c1)c1cc(Cl)ccc1O ; "and" ; O=C(Nc1cc(C(F)(F)F)cc(C(F)(F)F)c1)c1cc(Br)ccc1O]

1.8. Any of Method 1, 1.1-1.3, or 1.6 wherein R₆ is H or acetyl, e.g., H, e.g., acetyl.
1.9. Any of Method 1, 1.1-1.3, or 1.6 wherein R₁ is selected from chloro and bromo; R₃ and R₅ are both trifluoromethyl; and R₂ and R₄ are H and R₆ is acetyl, e.g., wherein the compound of Formula I is selected from:

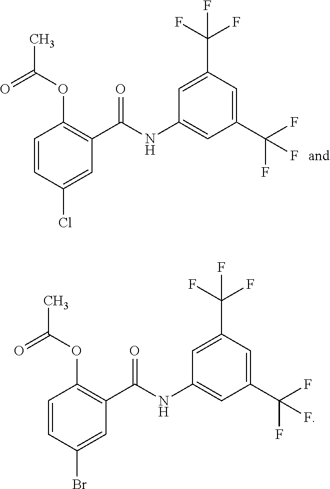 [SMILES: CC(=O)Oc1ccc(Cl)cc1C(=O)Nc1cc(C(F)(F)F)cc(C(F)(F)F)c1 ; "and" ; CC(=O)Oc1ccc(Br)cc1C(=O)Nc1cc(C(F)(F)F)cc(C(F)(F)F)c1]

1.10. Method 1.7 wherein the compound of Formula I is:

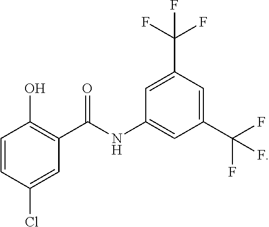 [SMILES: O=C(Nc1cc(C(F)(F)F)cc(C(F)(F)F)c1)c1cc(Cl)ccc1O]

1.11. Method 1.6 wherein R₁, R₃ and R₅ are each chloro, and R₂, R₄ and R₆ are each H.
1.12. Method 1.6 wherein R₁, R₃ and R₅ are each trifluoromethyl, and R₂, R₄ and R₆ are each H.
1.13. Any of Method 1, 1.1-1.3, or 1.6 wherein R₆ is C₁₋₄ acyl (e.g. acetyl).
1.14. Any of Method 1, 1.1-1.3, or 1.6 wherein R₆ is the residue of an amino acid.
1.15. Any of Method 1, 1.1-1.3, or 1.6 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
1.16. Any of Method 1, 1.1-1.3, or 1.6 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
1.17. Any of Method 1, 1.1-1.3, or 1.6 wherein R₆ is a (morpholin-4-yl)carbonyl group.
1.18. Any of Method 1, 1.1-1.3, or 1.6 wherein R₆ is a phosphono (—PO₃), which may be substituted, e.g. dibenzylphosphono (—P(═O)(OCH₂C₆H₅)₂), or unsubstituted (—P(═O)(OH)₂).
1.19. Method 1.18 wherein R₆ is —P(═O)(OH)₂.
1.20. Method 1.19 wherein the prodrug of Formula I is 2-((3,5-bis(trifluoromethyl)phenyl)carbamoyl)-4-chlorophenyl dihydrogen phosphate:

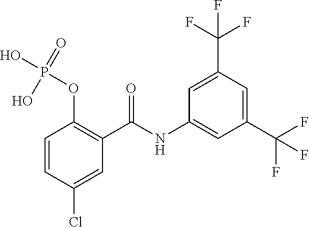 [SMILES: O=C(Nc1cc(C(F)(F)F)cc(C(F)(F)F)c1)c1cc(Cl)ccc1OP(=O)(O)O]

or a pharmaceutically acceptable salt thereof.
1.21. Method 1.20 comprising administering a pharmaceutically acceptable solution comprising a pharmaceutically acceptable salt of 2-((3,5-bis(trifluoromethyl)phenyl)carbamoyl)-4-chlorophenyl dihydrogen phosphate dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
1.22. Method 1.18 wherein the prodrug of Formula I is:

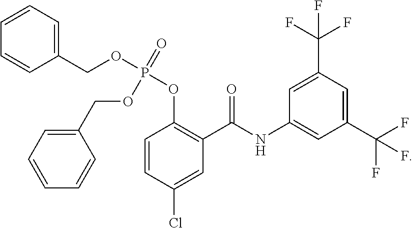 [SMILES: O=C(Nc1cc(C(F)(F)F)cc(C(F)(F)F)c1)c1cc(Cl)ccc1OP(=O)(OCc1ccccc1)OCc1ccccc1]

1.23. Method 1.18 wherein the pharmaceutically acceptable salt prodrug of Formula I is a compound of Formula Ia:

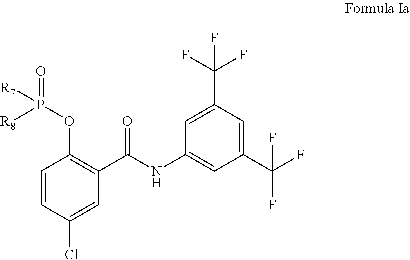

wherein one of R₇ and R₈ is OH and the other is O⁻Q⁺ or both R₇ and R₈ are O⁻Q⁺ wherein each Q⁺ is independently a pharmaceutically acceptable cation.
1.24. Method 1.23 wherein one of R₇ and R₈ is OH and the other is O⁻Q⁺.
1.25. Method 1.23 wherein both R₇ and R₈ are O⁻Q⁺.
1.26. Any of Method 1.23-1.25 wherein each Q⁺ is independently Na⁺ or K⁺.
1.27. Method 1.26 wherein each Q⁺ is N^{a}.
1.28. Method 1.27 wherein the pharmaceutically acceptable salt prodrug of Formula Ia is:

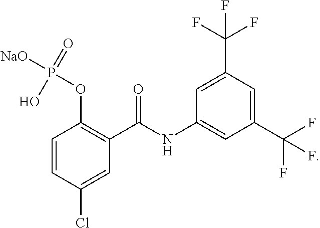

1.29. Method 1.27 wherein the pharmaceutically acceptable salt prodrug of Formula Ia is:

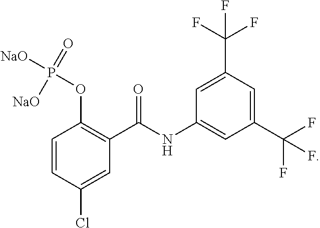

1.30. Any of Method 1.23-1.25 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂⁺, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃, ,,e.g., one R₉ is ,,CH₃ and another R₉ is —(CH₂)₆—) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
1.31. Method 1.30 wherein each Q⁺ is protonated tris(hydroxymethyl)aminomethane.
1.32. Any of Method 1.23-1.31 comprising administering a pharmaceutically acceptable solution comprising Formula Ia dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
1.33. Method 1.21 or 1.32 wherein the concentration of

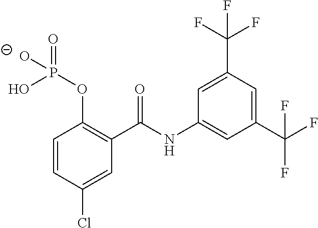 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
1.34. Method 1.21 or 1.32 wherein the concentration of

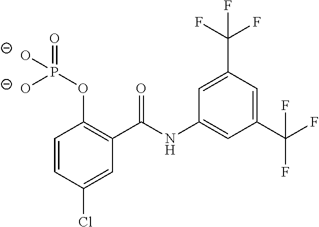 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
1.35. Any of Method 1 or 1.1-1.3 wherein the compound of Formula I is:

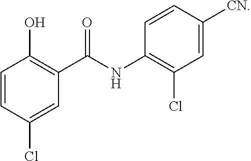 [SMILES: N#Cc1ccc(NC(=O)c2cc(Cl)ccc2O)c(Cl)c1]

1.36. Any of Method 1 or 1.1-1.3 wherein the phenylbenzamide is:

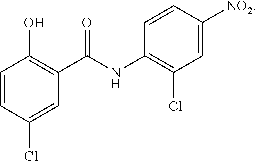 [SMILES: O=C(Nc1ccc([N+](=O)[O-])cc1Cl)c1cc(Cl)ccc1O]

1.37. Any of Method 1 et seq. comprising administering 0.1 or 0.25 mg to 2.0 g of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug thereof in an amount sufficient to provide 0.1 or 0.25 mg to 2.0 g of the phenylbenzamide, e.g., the compound of Formula I, e.g., N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
1.38. Any of Method 1 et seq. comprising administering 0.1 or 0.25 mg to 2.0 g of N-[3,5-bis(trifluoromethyl)phenyl]-5-chloro-2-hydroxybenzamide or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
1.39. Any of Method 1 et seq. comprising administering 0.1 or 0.25 mg to 2.0 g of

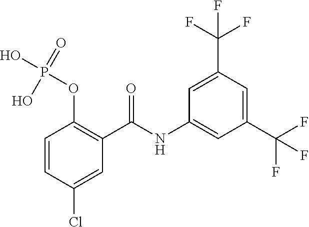 [SMILES: O=C(Nc1cc(C(F)(F)F)cc(C(F)(F)F)c1)c1cc(Cl)ccc1OP(=O)(O)O]

or a pharmaceutically acceptable salt thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering

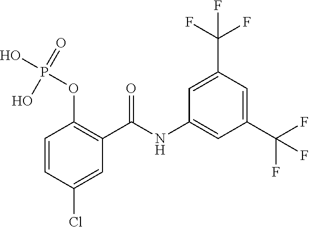 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
1.40. Any of Method 1 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising

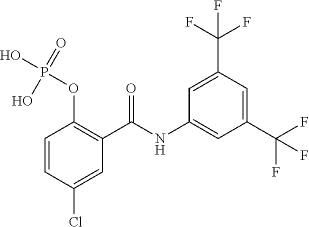 [SMILES: O=C(Nc1cc(C(F)(F)F)cc(C(F)(F)F)c1)c1cc(Cl)ccc1OP(=O)(O)O]

or a pharmaceutically acceptable salt thereof dissolved therein.
1.41. Any of Method 1 et seq. comprising administering 0.1 or 0.25 mg to 2.0 g of the compound of Formula Ia

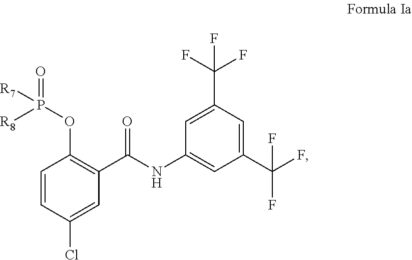

as described in any of Method 1.23-1.31, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the compound of Formula Ia

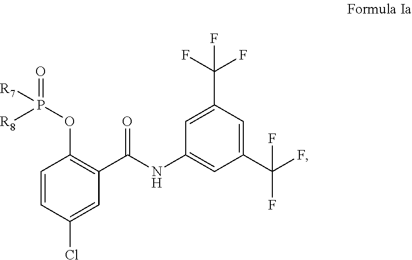

as described in any of Method 1.23-1.31, in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
1.42. Any of Method 1 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising Formula Ia

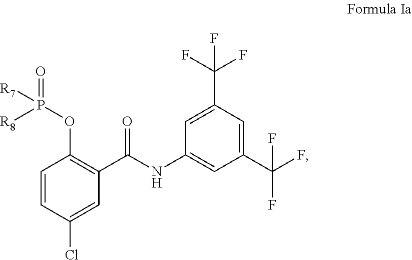

as described in any of Method 1.23-1.31, dissolved therein.
1.43. Any of Method 1.39-1.42 wherein the concentration of

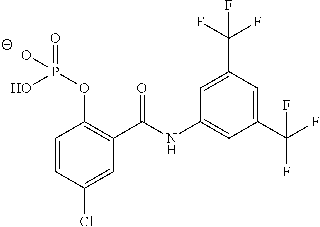 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
1.44. Any of Method 1.39-1.42 wherein the concentration of

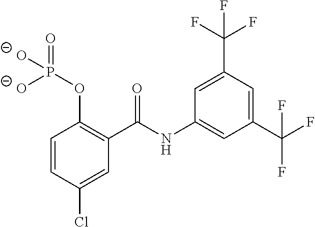 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
1.45. Any of Method 1 et seq. comprising administering a dose of 0.01 or 0.1 or 0.5 mg/kg to 1 or 5 or 10 or 15 mg/kg of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
1.46. Any of Method 1 et seq. comprising administering the pharmaceutically acceptable salt, prodrug, or pharmaceutically acceptable salt prodrug of Formula I, e.g., of N-[3,5-bis(trifluoromethyl)phenyl]-5-chloro-2-hydroxybenzamide, in an amount sufficient to provide a dose of 0.01 or 0.1 or 0.5 mg/kg to 1 or 5 or 10 or 15 mg/kg of the compound of Formula I, e.g., N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
1.47. Any of Method 1 et seq. comprising administering a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide or a pharmaceutically acceptable salt, prodrug (e.g., 2-((3,5-bis(trifluoromethyl)phenyl)carbamoyl)-4-chlorophenyl dihydrogen phosphate), or pharmaceutically acceptable salt prodrug thereof (e.g., Formula Ia as described in any of Method 1.23-1.31), e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
1.48. Any of Method 1 et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 1.23-1.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
1.49. Any of Method 1 et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 1.23-1.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
1.50. Any of Method 1 et seq. comprising administering

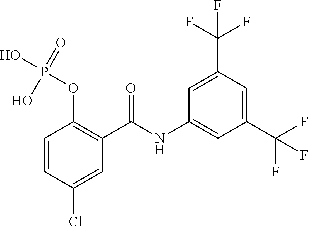 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
1.51. Any of Method 1 et seq. comprising administering

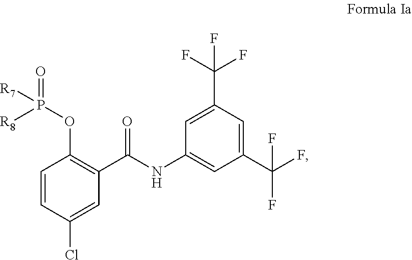

as described in any of Method 1.23-1.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
1.52. Any of Method 1 et seq. wherein the rejection or edema, e.g., transplant rejection, e.g., edema, is consequent to an autograft.
1.53. Any of Method 1 or 1.1-1.51 wherein the rejection or edema, e.g., transplant rejection, e.g., edema, is consequent to a syngeneic graft.
1.54. Any of Method 1 or 1.1-1.51 wherein the rejection or edema, e.g., transplant rejection, e.g., edema, is consequent to an isograft.
1.55. Any of Method 1 or 1.1-1.51 wherein the rejection or edema, e.g., transplant rejection, e.g., edema, is consequent to an allograft.
1.56. Any of Method 1 or 1.1-1.51 wherein the rejection or edema, e.g., transplant rejection, e.g., edema, is consequent to a xenograft.
1.57. Any of Method 1 et seq. wherein the rejection or edema, e.g., transplant rejection, e.g., edema, is consequent to a cell transplant, e.g., hematopoietic stem cell transplant, lymphocyte transplant, or pancreatic islet cell transplant, e.g., hematopoietic stem cell transplant, e.g., lymphocyte transplant, e.g., pancreatic islet cell transplant.
1.58. Any of Method 1 or 1.1-1.56 wherein the rejection or edema, e.g., transplant rejection, e.g., edema, is consequent to a tissue transplant.
1.59. Method 1.58 wherein the tissue is bone, tendon, cartilage, connective tissue, skin, cornea, sclera, heart valve, nerve, or vessel.
1.60. Any of Method 1 or 1.1-1.56 wherein the rejection or edema, e.g., transplant rejection, e.g., edema, is consequent to transplant of an organ or a portion thereof.
1.61. Method 1.60 wherein the organ is a kidney.
1.62. Method 1.60 wherein the organ is the liver.
1.63. Method 1.60 wherein the organ is the pancreas.
1.64. Method 1.60 wherein the organ is a lung.
1.65. Method 1.60 wherein the organ is the heart.
1.66. Method 1.60 wherein the organ is the thymus.
1.67. Method 1.60 wherein the organ is the intestine.
1.68. Method 1.60 wherein the organ is the uterus.
1.69. Any of Method 1 or 1.1-1.56 wherein the rejection or edema, e.g., transplant rejection, e.g., edema, is consequent to a face, limb (e.g., hand), eye, trachea, muscle, or esophagus transplant.
1.70. Any of Method 1 et seq. wherein the transplant rejection is hyperacute or accelerated rejection, e.g., hyperacute rejection, e.g., accelerated rejection.
1.71. Any of Method 1 or 1.1-1.69 wherein the transplant rejection is acute rejection.
1.72. Any of Method 1 or 1.1-1.69 wherein the transplant rejection is chronic rejection.
1.73. Any of Method 1 et seq. wherein the aquaporin is AQP4.
1.74. Any of Method 1 et seq. wherein the aquaporin is AQP2.
1.75. Any of Method 1 et seq. wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered orally, e.g., tablet, capsule, solution, suspension, or the like.
1.76. Method 1.75 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method
1.20, 1.21, or 1.23-1.34, is administered orally.
1.77. Any of Method 1 or 1.1-1.74 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered parenterally.
1.78. Method 1.77 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method
1.20, 1.21, or 1.23-1.34, is administered parenterally.
1.79. Method 1.77 or 1.78 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
1.80. Any of Method 1.77-1.79 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method
1.20, 1.21, or 1.23-1.34, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
1.81. Any of Method 1.77-1.80 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intravenously, e.g., IV bolus and/or IV infusion, e.g., IV bolus followed by IV infusion.
1.82. Any of Method 1.77-1.81 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method
1.20, 1.21, or 1.23-1.34, is administered intravenously, e.g., IV bolus and/or IV infusion, e.g., IV bolus followed by IV infusion.
1.83. Any of Method 1.77-1.80 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intramuscularly, e.g., IM bolus and/or IM infusion, e.g., IM bolus followed by IM infusion.
1.84. Any of Method 1.77-1.80 or 1.83 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method
1.20, 1.21, or 1.23-1.34, is administered intramuscularly, e.g., IM bolus and/or IM infusion, e.g., IM bolus followed by IM infusion.
1.85. Any of Method 1.77-1.84 wherein the infusion, e.g., IV or IM, is administered over 10 or 30 minutes to 72 hours, e.g., 30 minutes to 24 hours, e.g., 30 minutes to 12 hours, e.g., 30 minutes to 8 hours, e.g., 30 minutes to 6 hours, e.g., 30 minutes to 4 hours, e.g., 30 minutes to 2 hours, e.g., 30 minutes to 1 hour.
1.86. Any of Method 1 et seq. comprising concurrently or sequentially administering another treatment for transplant rejection.
1.87. Any of Method 1 et seq. comprising concurrently or sequentially administering an immunosuppressant (e.g., a corticosteroid (e.g., prednisone, prednisolone, methylprednisolone, hydrocortisone, dexamethasone), a calcineurin inhibitor (e.g., cyclosporine, tacrolimus), a purine metabolism inhibitor (e.g., azathioprine, mycophenolate mofetil), a rapamycin (e.g., sirolimus, everolimus), an immunosuppressive Ig (e.g., antilymphocyte globulin, antithymocyte globulin, anti-Tac antibody), a monoclonal antibody (mAb) (e.g., OKT3, an anti-IL-2 receptor monoclonal antibody (e.g., basiliximab, daclizumab)), or an agent that inhibits T-cell costimulatory pathways (e.g., a cytotoxic T lymphocyte-associated antigen 4 (CTLA-4)-IgG1 fusion protein, belatacept), or a combination thereof.
1.88. Any of Method 1 et seq. further comprising induction of chimerism using nonmyeloablative pretransplantation treatment (e.g., with cyclophosphamide, thymic irradiation, antithymocte globulin, or cyclosporin, or a combination thereof).
1.89. Any of Method 1 et seq. further comprising total body irradiation.
1.90. Any of Method 1 et seq. wherein the patient is human.
1.91. Any of Method 1 et seq. wherein the onset of action of any of the compounds identified in any of Methods 1, 1.6-1.20, or 1.22-1.31, is fairly rapid.
1.92. Any of Method 1 et seq. comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 1.20, 1.21, or 1.23-1.34, prior to transplantation, e.g., 12 hours or less, e.g. 8 hours or less, e.g., 6 hours or less, e.g., 3 hours or less, e.g., 2 hours or less, e.g., 1 hour or less, e.g., 30 minutes or less, e.g., 10 or 5 minutes or less, prior to transplantation.
1.93. Any of Method 1 et seq. comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 1.20, 1.21, or 1.23-1.34, contemporaneously with transplantation.
1.94. Any of Method 1 et seq. comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 1.20, 1.21, or 1.23-1.34, after transplantation.
1.95. Method 1.94 wherein the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 1.20, 1.21, or 1.23-1.34, e.g., comprising administering a pharmaceutically acceptable solution prepared by dissolving 2-((3,5-bis(trifluoromethyl)phenyl)carbamoyl)-4-chlorophenyl dihydrogen phosphate and tris(hydroxymethyl)aminomethane, is administered for 6 months or less after the transplant, e.g., 5 months or less, e.g., 4 months or less, e.g., 3 months or less, e.g., 2 months or less, e.g., 1 month or less, e.g., 3 weeks or less, e.g., 2 weeks or less, e.g., 1 week or less.
1.96. Any of Method 1 et seq. wherein the patient is a transplant donor.
1.97. Any of Method 1 or 1.1-1.95 wherein the patient is a transplant recipient.

In another embodiment, provided is a method (Method 2) for treatment or prophylaxis of transplant rejection, inhibiting rejection of transplanted biological material, or prophylaxis, treatment, or control of edema consequent to a transplant, comprising administering to a patient in need thereof an effective amount of an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I:

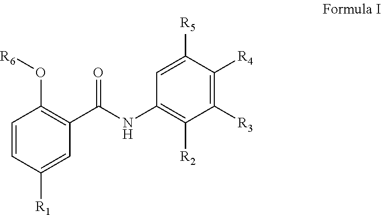

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
2.1. Method 2 comprising treatment or prophylaxis of transplant rejection.
2.2. Method 2 comprising inhibiting rejection of transplanted biological material.
2.3. Method 2 comprising prophylaxis, treatment, or control of edema consequent to a transplant.
2.4. Any of Method 2 et seq. wherein the phenylbenzamide is as described in U.S. Patent Publication No. 2010/0274051.
2.5. Any of Method 2 et seq. wherein the phenylbenzamide is as described in U.S. Pat. Nos. 7,626,042 or 7,700,655.
2.6. Any of Method 2 or 2.1-2.3 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
2.7. Method 2.6 wherein R₁ is selected from chloro and bromo; R₃ and R₅ are both trifluoromethyl; and R₂, R₄ and R₆ are all H, e.g., wherein the compound of Formula I is selected from:

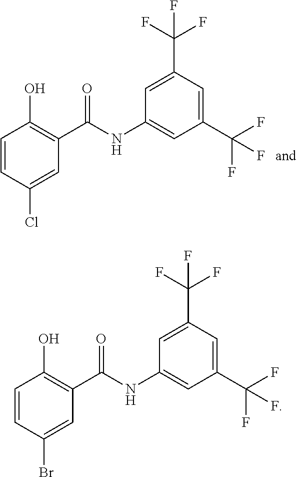 [SMILES: O=C(Nc1cc(C(F)(F)F)cc(C(F)(F)F)c1)c1cc(Cl)ccc1O ; "and" ; O=C(Nc1cc(C(F)(F)F)cc(C(F)(F)F)c1)c1cc(Br)ccc1O]

2.8. Any of Method 2, 2.1-2.3, or 2.6 wherein R₆ is H or acetyl, e.g., H, e.g., acetyl.
2.9. Any of Method 2, 2.1-2.3, or 2.6 wherein R₁ is selected from chloro and bromo; R₃ and R₅ are both trifluoromethyl; and R₂ and R₄ are H and R₆ is acetyl, e.g., wherein the compound of Formula I is selected from:

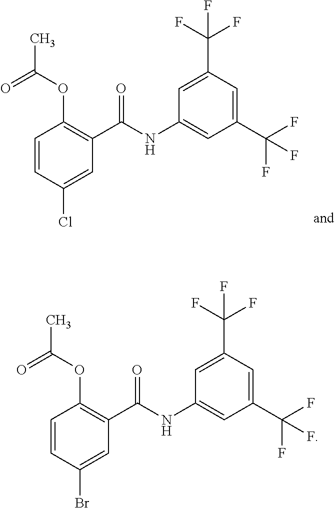 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

2.10. Method 2.7 wherein the compound of Formula I is:

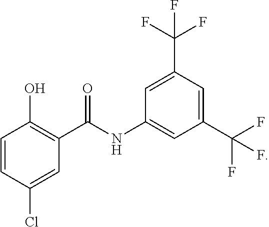 [SMILES: O=C(Nc1cc(C(F)(F)F)cc(C(F)(F)F)c1)c1cc(Cl)ccc1O]

2.11. Method 2.6 wherein R₁, R₃ and R₅ are each chloro, and R₂, R₄ and R₆ are each H.
2.12. Method 2.6 wherein R₁, R₃ and R₅ are each trifluoromethyl, and R₂, R₄ and R₆ are each H.
2.13. Any of Method 2, 2.1-2.3, or 2.6 wherein R₆ is C₁₋₄ acyl (e.g. acetyl).
2.14. Any of Method 2, 2.1-2.3, or 2.6 wherein R₆ is the residue of an amino acid.
2.15. Any of Method 2, 2.1-2.3, or 2.6 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
2.16. Any of Method 2, 2.1-2.3, or 2.6 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
2.17. Any of Method 2, 2.1-2.3, or 2.6 wherein R₆ is a (morpholin-4-yl)carbonyl group.
2.18. Any of Method 2, 2.1-2.3, or 2.6 wherein R₆ is a phosphono (—PO₃), which may be substituted, e.g. dibenzylphosphono (—P(═O)(OCH₂C₆H₅)₂), or unsubstituted (—P(═O)(OH)₂).
2.19. Method 2.18 wherein R₆ is ,,P(═O)(OH)₂.
2.20. Method 2.19 wherein the prodrug of Formula I is 2-((3,5-bis(trifluoromethyl)phenyl)carbamoyl)-4-chlorophenyl dihydrogen phosphate:

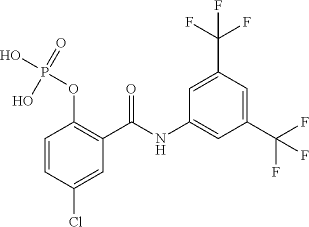 [SMILES: O=C(Nc1cc(C(F)(F)F)cc(C(F)(F)F)c1)c1cc(Cl)ccc1OP(=O)(O)O]

or a pharmaceutically acceptable salt thereof.
2.21. Method 2.20 comprising administering a pharmaceutically acceptable solution comprising a pharmaceutically acceptable salt of 2-((3,5-bis(trifluoromethyl)phenyl)carbamoyl)-4-chlorophenyl dihydrogen phosphate dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
2.22. Method 2.18 wherein the prodrug of Formula I is:

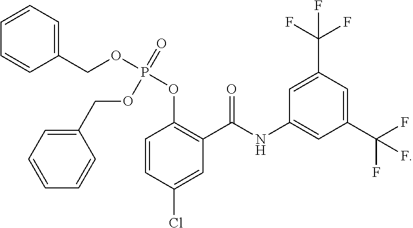 [SMILES: O=C(Nc1cc(C(F)(F)F)cc(C(F)(F)F)c1)c1cc(Cl)ccc1OP(=O)(OCc1ccccc1)OCc1ccccc1]

2.23. Method 2.18 wherein the pharmaceutically acceptable salt prodrug of Formula I is a compound of Formula Ia:

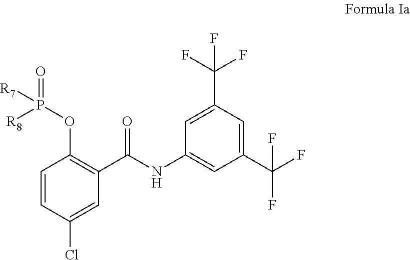

wherein one of R₇ and R₈ is OH and the other is O⁻Q⁺ or both R₇ and R₈ are O⁻Q⁺ wherein each Q⁺ is independently a pharmaceutically acceptable cation.
2.24. Method 2.23 wherein one of R₇ and R₈ is OH and the other is O⁻Q⁺.
2.25. Method 2.23 wherein both R₇ and R₈ are O⁻Q⁺.
2.26. Any of Method 2.23-2.25 wherein each Q⁺ is independently Na⁺ or K⁺.
2.27. Method 2.26 wherein each Q⁺ is Na⁺.
2.28. Method 2.27 wherein the pharmaceutically acceptable salt prodrug of Formula Ia is:

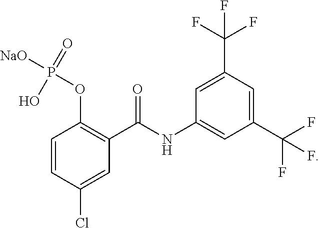

2.29. Method 2.27 wherein the pharmaceutically acceptable salt prodrug of Formula Ia is:

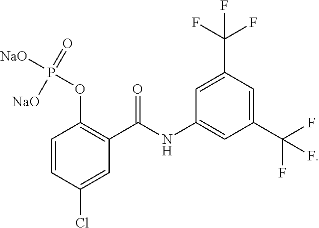

2.30. Any of Method 2.23-2.25 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂⁺, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,, CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
2.31. Method 2.30 wherein each Q⁺ is protonated tris(hydroxymethyl)aminomethane.
2.32. Any of Method 2.23-2.31 comprising administering a pharmaceutically acceptable solution comprising Formula Ia dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
2.33. Method 2.21 or 2.32 wherein the concentration of

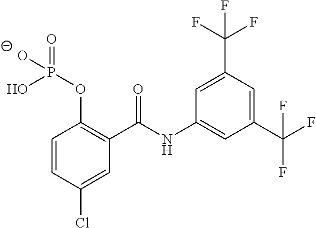 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
2.34. Method 2.21 or 2.32 wherein the concentration of

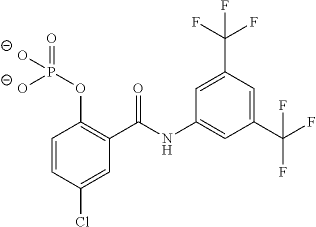 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
2.35. Any of Method 2 or 2.1-2.3 wherein the compound of Formula I is:

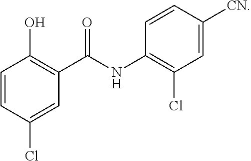 [SMILES: N#Cc1ccc(NC(=O)c2cc(Cl)ccc2O)c(Cl)c1]

2.36. Any of Method 2 or 2.1-2.3 wherein the phenylbenzamide is:

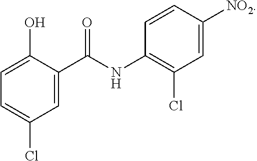 [SMILES: O=C(Nc1ccc([N+](=O)[O-])cc1Cl)c1cc(Cl)ccc1O]

2.37. Any of Method 2 et seq. comprising administering 0.1 or 0.25 mg to 2.0 g of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug thereof in an amount sufficient to provide 0.1 or 0.25 mg to 2.0 g of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
2.38. Any of Method 2 et seq. comprising administering 0.1 or 0.25 mg to 2.0 g of N-[3,5-bis(trifluoromethyl)phenyl]-5-chloro-2-hydroxybenzamide or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
2.39. Any of Method 2 et seq. comprising administering 0.1 or 0.25 mg to 2.0 g of

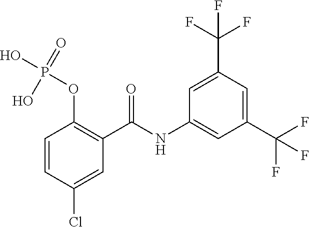 [SMILES: O=C(Nc1cc(C(F)(F)F)cc(C(F)(F)F)c1)c1cc(Cl)ccc1OP(=O)(O)O]

or a pharmaceutically acceptable salt thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering

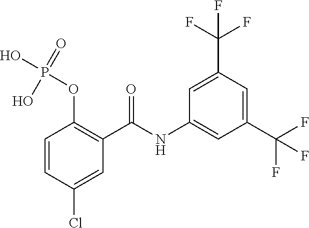 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
2.40. Any of Method 2 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising

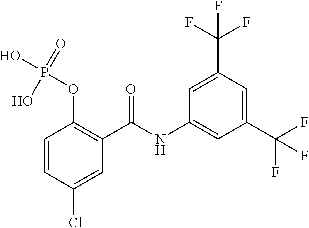 [SMILES: O=C(Nc1cc(C(F)(F)F)cc(C(F)(F)F)c1)c1cc(Cl)ccc1OP(=O)(O)O]

or a pharmaceutically acceptable salt thereof dissolved therein.
2.41. Any of Method 2 et seq. comprising administering 0.1 or 0.25 mg to 2.0 g of the compound of Formula Ia

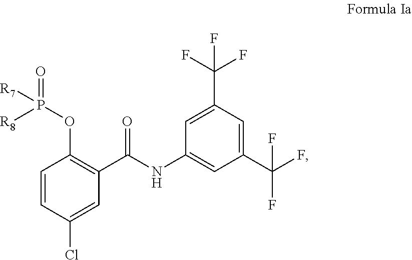

as described in any of Method 2.23-2.31, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the compound of Formula Ia

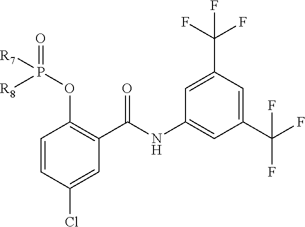

as described in any of Method 2.23-2.31, in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
2.42. Any of Method 2 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising Formula Ia

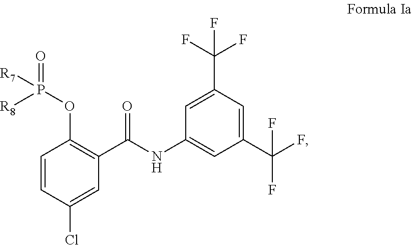

as described in any of Method 2.23-2.31, dissolved therein.
2.43. Any of Method 2.39-2.42 wherein the concentration of

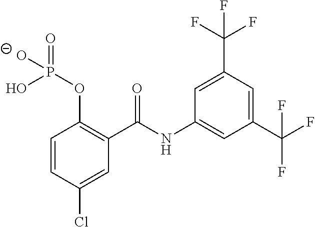 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
2.44. Any of Method 2.39-2.42 wherein the concentration of

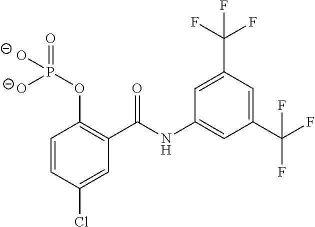 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
2.45. Any of Method 2 et seq. comprising administering a dose of 0.01 or 0.1 or 0.5 mg/kg to 1 or 5 or 10 or 15 mg/kg of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
2.46. Any of Method 2 et seq. comprising administering the pharmaceutically acceptable salt, prodrug, or pharmaceutically acceptable salt prodrug of Formula I, e.g., of N-[3,5-bis(trifluoromethyl)phenyl]-5-chloro-2-hydroxybenzamide, in an amount sufficient to provide a dose of 0.01 or 0.1 or 0.5 mg/kg to 1 or 5 or 10 or 15 mg/kg of the compound of Formula I, e.g., N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
2.47. Any of Method 2 et seq. comprising administering a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide or a pharmaceutically acceptable salt, prodrug (e.g., 2-((3,5-bis(trifluoromethyl)phenyl)carbamoyl)-4-chlorophenyl dihydrogen phosphate), or pharmaceutically acceptable salt prodrug thereof (e.g., Formula Ia as described in any of Method 2.23-2.31), e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
2.48. Any of Method 2 et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 2.23-2.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
2.49. Any of Method 2 et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 2.23-2.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
2.50. Any of Method 2 et seq. comprising administering

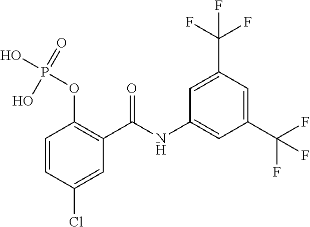 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
2.51. Any of Method 2 et seq. comprising administering

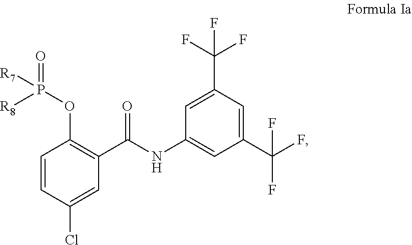

as described in any of Method 2.23-2.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
2.52. Any of Method 2 et seq. wherein the rejection or edema, e.g., transplant rejection, e.g., edema, is consequent to an autograft.
2.53. Any of Method 2 or 2.1-2.51 wherein the rejection or edema, e.g., transplant rejection, e.g., edema, is consequent to a syngeneic graft.
2.54. Any of Method 2 or 2.1-2.51 wherein the rejection or edema, e.g., transplant rejection, e.g., edema, is consequent to an isograft.
2.55. Any of Method 2 or 2.1-2.51 wherein the rejection or edema, e.g., transplant rejection, e.g., edema, is consequent to an allograft.
2.56. Any of Method 2 or 2.1-2.51 wherein the rejection or edema, e.g., transplant rejection, e.g., edema, is consequent to a xenograft.
2.57. Any of Method 2 et seq. wherein the rejection or edema, e.g., transplant rejection, e.g., edema, is consequent to a cell transplant, e.g., hematopoietic stem cell transplant, lymphocyte transplant, or pancreatic islet cell transplant, e.g., hematopoietic stem cell transplant, e.g., lymphocyte transplant, e.g., pancreatic islet cell transplant.
2.58. Any of Method 2 or 2.1-2.56 wherein the rejection or edema, e.g., transplant rejection, e.g., edema, is consequent to a tissue transplant.
2.59. Method 2.58 wherein the tissue is bone, tendon, cartilage, connective tissue, skin, cornea, sclera, heart valve, nerve, or vessel.
2.60. Any of Method 2 or 2.1-2.56 wherein the rejection or edema, e.g., transplant rejection, e.g., edema, is consequent to transplant of an organ or a portion thereof.
2.61. Method 2.60 wherein the organ is a kidney.
2.62. Method 2.60 wherein the organ is the liver.
2.63. Method 2.60 wherein the organ is the pancreas.
2.64. Method 2.60 wherein the organ is a lung.
2.65. Method 2.60 wherein the organ is the heart.
2.66. Method 2.60 wherein the organ is the thymus.
2.67. Method 2.60 wherein the organ is the intestine.
2.68. Method 2.60 wherein the organ is the uterus.
2.69. Any of Method 2 or 2.1-2.56 wherein the rejection or edema, e.g., transplant rejection, e.g., edema, is consequent to a face, limb (e.g., hand), eye, trachea, muscle, or esophagus transplant.
2.70. Any of Method 2 et seq. wherein the transplant rejection is hyperacute or accelerated rejection, e.g., hyperacute rejection, e.g., accelerated rejection.
2.71. Any of Method 2 or 2.1-2.69 wherein the transplant rejection is acute rejection.
2.72. Any of Method 2 or 2.1-2.69 wherein the transplant rejection is chronic rejection.
2.73. Any of Method 2 et seq. wherein the aquaporin is AQP4.
2.74. Any of Method 2 et seq. wherein the aquaporin is AQP2.
2.75. Any of Method 2 et seq. wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered orally, e.g., tablet, capsule, solution, suspension, or the like.
2.76. Method 2.75 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 2.20, 2.21, or 2.23-2.34, is administered orally.
2.77. Any of Method 2 or 2.1-2.74 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered parenterally.
2.78. Method 2.77 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 2.20, 2.21, or 2.23-2.34, is administered parenterally.
2.79. Method 2.77 or 2.78 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
2.80. Any of Method 2.77-2.79 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 2.20, 2.21, or 2.23-2.34, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
2.81. Any of Method 2.77-2.80 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intravenously, e.g., IV bolus and/or IV infusion, e.g., IV bolus followed by IV infusion.
2.82. Any of Method 2.77-2.81 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 2.20, 2.21, or 2.23-2.34, is administered intravenously, e.g., IV bolus and/or IV infusion, e.g., IV bolus followed by IV infusion.
2.83. Any of Method 2.77-2.80 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intramuscularly, e.g., IM bolus and/or IM infusion, e.g., IM bolus followed by IM infusion.
2.84. Any of Method 2.77-2.81 or 2.83 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 2.20, 2.21, or 2.23-2.34, is administered intramuscularly, e.g., IM bolus and/or IM infusion, e.g., IM bolus followed by IM infusion.
2.85. Any of Method 2.77-2.84 wherein the infusion, e.g., IV or IM, is administered over 10 or 30 minutes to 72 hours, e.g., 30 minutes to 24 hours, e.g., 30 minutes to 12 hours, e.g., 30 minutes to 8 hours, e.g., 30 minutes to 6 hours, e.g., 30 minutes to 4 hours, e.g., 30 minutes to 2 hours, e.g., 30 minutes to 1 hour.
2.86. Any of Method 2 et seq. comprising concurrently or sequentially administering another treatment for transplant rejection.
2.87. Any of Method 2 et seq. comprising concurrently or sequentially administering an immunosuppressant (e.g., a corticosteroid (e.g., prednisone, prednisolone, methylprednisolone, hydrocortisone, dexamethasone), a calcineurin inhibitor (e.g., cyclosporine, tacrolimus), a purine metabolism inhibitor (e.g., azathioprine, mycophenolate mofetil), a rapamycin (e.g., sirolimus, everolimus), an immunosuppressive Ig (e.g., antilymphocyte globulin, antithymocyte globulin, anti-Tac antibody), a monoclonal antibody (mAb) (e.g., OKT3, an anti-IL-2 receptor monoclonal antibody (e.g., basiliximab, daclizumab)), or an agent that inhibits T-cell costimulatory pathways (e.g., a cytotoxic T lymphocyte-associated antigen 4 (CTLA-4)-IgG1 fusion protein, belatacept), or a combination thereof.
2.88. Any of Method 2 et seq. further comprising induction of chimerism using nonmyeloablative pretransplantation treatment (e.g., with cyclophosphamide, thymic irradiation, antithymocte globulin, or cyclosporin, or a combination thereof).
2.89. Any of Method 2 et seq. further comprising total body irradiation.
2.90. Any of Method 2 et seq. wherein the patient is human.
2.91. Any of Method 2 et seq. wherein the onset of action of any of the compounds identified in any of Methods 2, 2.6-2.20, or 2.22-2.31, is fairly rapid.
2.92. Any of Method 2 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 2.20, 2.21, or 2.23-2.34, prior to transplantation, e.g., 12 hours or less, e.g. 8 hours or less, e.g., 6 hours or less, e.g., 3 hours or less, e.g., 2 hours or less, e.g., 1 hour or less, e.g., 30 minutes or less, e.g., 10 or 5 minutes or less, prior to transplantation.
2.93. Any of Method 2 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 2.20, 2.21, or 2.23-2.34, contemporaneously with transplantation.
2.94. Any of Method 2 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 2.20, 2.21, or 2.23-2.34, after transplantation.
2.95. Method 2.94 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 2.20, 2.21, or 2.23-2.34, e.g., comprising administering a pharmaceutically acceptable solution prepared by dissolving 2-((3,5-bis(trifluoromethyl)phenyl)carbamoyl)-4-chlorophenyl dihydrogen phosphate and tris(hydroxymethyl)aminomethane, is administered for 6 months or less after the transplant, e.g., 5 months or less, e.g., 4 months or less, e.g., 3 months or less, e.g., 2 months or less, e.g., 1 month or less, e.g., 3 weeks or less, e.g., 2 weeks or less, e.g., 1 week or less.
2.96. Any of Method 2 et seq. wherein the patient is a transplant donor.
2.97. Any of Method 2 et seq. wherein the patient is a transplant recipient.
2.98. Any of Method 2 et seq. wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 2.20, 2.21, or 2.23-2.34, e.g., is administered before and/or after the transplant.

In one embodiment, provided is a method (Method 3) for treatment or prophylaxis of transplant rejection, inhibiting rejection of transplanted biological material, or prophylaxis, treatment, or control of edema consequent to a transplant, comprising administering to a patient in need thereof an aquaporin inhibitor, e.g., a compound binding to an aquaporin, e.g. AQP4, e.g., an inhibitor of AQP2 or AQP4, e.g., AQP4, in an amount effective to inhibit the aquaporin, e.g., AQP4, wherein the aquaporin inhibitor is a phenylbenzamide, e.g., a compound of Formula I:

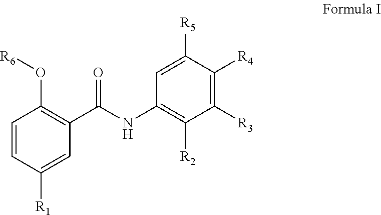

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
3.1. Method 3 comprising treatment or prophylaxis of transplant rejection
3.2. Method 3 comprising inhibiting rejection of transplanted biological material.
3.3. Method 3 comprising prophylaxis, treatment, or control of edema consequent to a transplant.
3.4. Any of Method 3 et seq. wherein the phenylbenzamide is as described in U.S. Patent Publication No. 2010/0274051.
3.5. Any of Method 3 et seq. wherein the phenylbenzamide is as described in U.S. Pat. Nos. 7,626,042 or 7,700,655.
3.6. Any of Method 3 or 3.1-3.3 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
3.7. Method 3.6 wherein R₁ is selected from chloro and bromo; R₃ and R₅ are both trifluoromethyl; and R₂, R₄ and R₆ are all H, e.g., wherein the compound of Formula I is selected from:

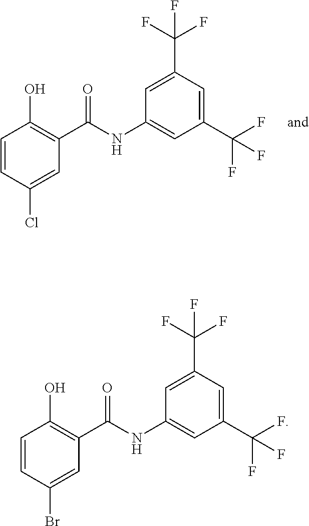 [SMILES: O=C(Nc1cc(C(F)(F)F)cc(C(F)(F)F)c1)c1cc(Cl)ccc1O ; O=C(Nc1cc(C(F)(F)F)cc(C(F)(F)F)c1)c1cc(Br)ccc1O]

3.8. Any of Method 3, 3.1-3.3, or 3.6 wherein R₆ is H or acetyl, e.g., H, e.g., acetyl.
3.9. Any of Method 3, 3.1-3.3, or 3.6 wherein R₁ is selected from chloro and bromo; R₃ and R₅ are both trifluoromethyl; and R₂ and R₄ are H and R₆ is acetyl, e.g., wherein the compound of Formula I is selected from:

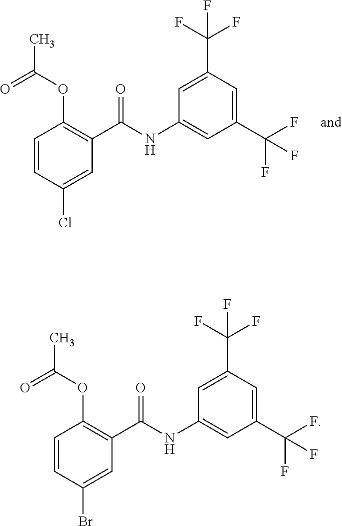 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

3.10. Method 3.7 wherein the compound of Formula I is:

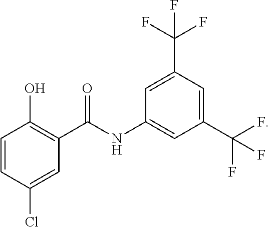 [SMILES: O=C(Nc1cc(C(F)(F)F)cc(C(F)(F)F)c1)c1cc(Cl)ccc1O]

3.11. Method 3.6 wherein R₁, R₃ and R₅ are each chloro, and R₂, R₄ and R₆ are each H.
3.12. Method 3.6 wherein R₁, R₃ and R₅ are each trifluoromethyl, and R₂, R₄ and R₆ are each H.
3.13. Any of Method 3, 3.1-3.3, or 3.6 wherein R₆ is C₁₋₄ acyl (e.g. acetyl).
3.14. Any of Method 3, 3.1-3.3, or 3.6 wherein R₆ is the residue of an amino acid.
3.15. Any of Method 3, 3.1-3.3, or 3.6 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
3.16. Any of Method 3, 3.1-3.3, or 3.6 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
3.17. Any of Method 3, 3.1-3.3, or 3.6 wherein R₆ is a (morpholin-4-yl)carbonyl group.
3.18. Any of Method 3, 3.1-3.3, or 3.6 wherein R₆ is a phosphono (—PO₃), which may be substituted, e.g. dibenzylphosphono (—P(═O)(OCH₂C₆H₅)₂), or unsubstituted (—P(═O)(OH)₂).
3.19. Method 3.18 wherein R₆ is ,,P(═O)(OH)₂.
3.20. Method 3.19 wherein the prodrug of Formula I is 2-((3,5-bis(trifluoromethyl)phenyl)carbamoyl)-4-chlorophenyl dihydrogen phosphate:

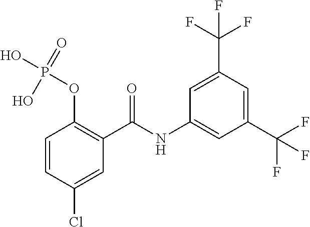 [SMILES: O=C(Nc1cc(C(F)(F)F)cc(C(F)(F)F)c1)c1cc(Cl)ccc1OP(=O)(O)O]

or a pharmaceutically acceptable salt thereof.
3.21. Method 3.20 comprising administering a pharmaceutically acceptable solution comprising a pharmaceutically acceptable salt of 2-((3,5-bis(trifluoromethyl)phenyl)carbamoyl)-4-chlorophenyl dihydrogen phosphate dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
3.22. Method 3.18 wherein the prodrug of Formula I is:

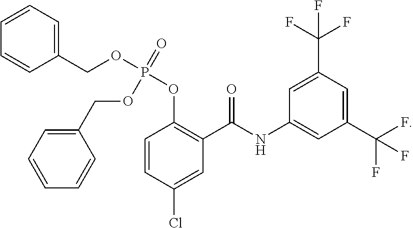 [SMILES: O=C(Nc1cc(C(F)(F)F)cc(C(F)(F)F)c1)c1cc(Cl)ccc1OP(=O)(OCc1ccccc1)OCc1ccccc1]

3.23. Method 3.18 wherein the pharmaceutically acceptable salt prodrug of Formula I is a compound of Formula Ia:

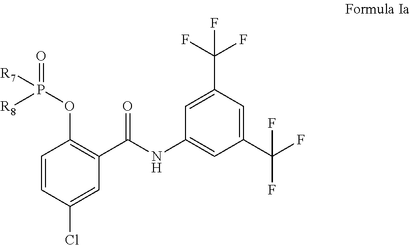

wherein one of R₇ and R₈ is OH and the other is O⁻Q⁺ or both R₇ and R₈ are O⁻Q⁺ wherein each Q⁺ is independently a pharmaceutically acceptable cation.
3.24. Method 3.23 wherein one of R₇ and R₈ is OH and the other is O⁻Q⁺.
3.25. Method 3.23 wherein both R₇ and R₈ are O⁻Q⁺.
3.26. Any of Method 3.23-3.25 wherein each Q⁺ is independently Na⁺ or K⁺.
3.27. Method 3.26 wherein each Q⁺ is Na⁺.
3.28. Method 3.27 wherein the pharmaceutically acceptable salt prodrug of Formula Ia is:

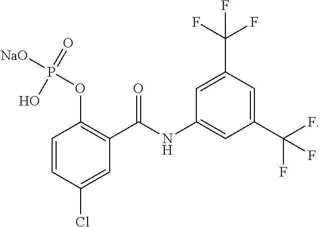

3.29. Method 3.27 wherein the pharmaceutically acceptable salt prodrug of Formula Ia is:

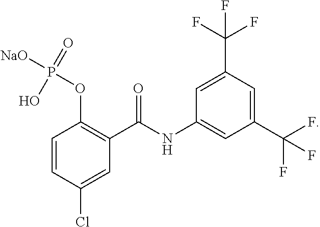

3.30. Any of Method 3.23-3.25 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃, [(HO)ₙR₉]₂NH₂, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
3.31. Method 3.30 wherein each Q⁺ is protonated tris(hydroxymethyl)aminomethane.
3.32. Any of Method 3.23-3.31 comprising administering a pharmaceutically acceptable solution comprising Formula Ia dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
3.33. Method 3.21 or 3.32 wherein the concentration of

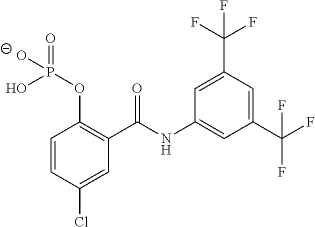 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
3.34. Method 3.21 or 3.32 wherein the concentration of

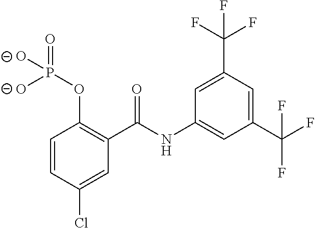 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
3.35. Any of Method 3 or 3.1-3.3 wherein the compound of Formula I is:

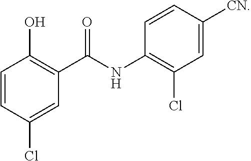 [SMILES: N#Cc1ccc(NC(=O)c2cc(Cl)ccc2O)c(Cl)c1]

3.36. Any of Method 3 or 3.1-3.3 wherein the phenylbenzamide is:

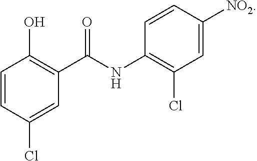 [SMILES: O=C(Nc1ccc([N+](=O)[O-])cc1Cl)c1cc(Cl)ccc1O]

3.37. Any of Method 3 et seq. comprising administering 0.1 or 0.25 mg to 2.0 g of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug thereof in an amount sufficient to provide 0.1 or 0.25 mg to 2.0 g of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
3.38. Any of Method 3 et seq. comprising administering 0.1 or 0.25 mg to 2.0 g of N-[3,5-bis(trifluoromethyl)phenyl]-5-chloro-2-hydroxybenzamide or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
3.39. Any of Method 3 et seq. comprising administering 0.1 or 0.25 mg to 2.0 g of

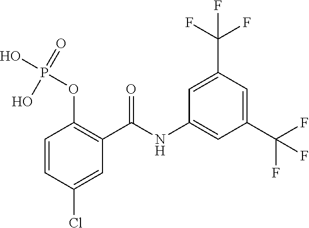 [SMILES: O=C(Nc1cc(C(F)(F)F)cc(C(F)(F)F)c1)c1cc(Cl)ccc1OP(=O)(O)O]

or a pharmaceutically acceptable salt thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering

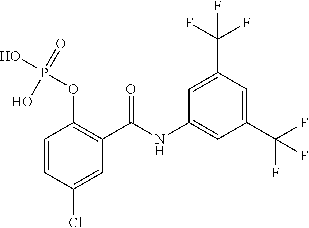 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide 0.1 or 0.25 mg to 2.0 g ofN-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
3.40. Any of Method 3 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising

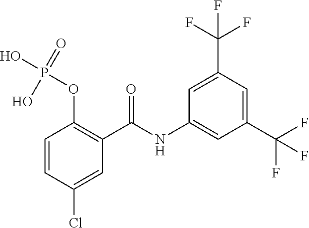 [SMILES: O=C(Nc1cc(C(F)(F)F)cc(C(F)(F)F)c1)c1cc(Cl)ccc1OP(=O)(O)O]

or a pharmaceutically acceptable salt thereof dissolved therein.
3.41. Any of Method 3 et seq. comprising administering 0.1 or 0.25 mg to 2.0 g of the compound of Formula Ia

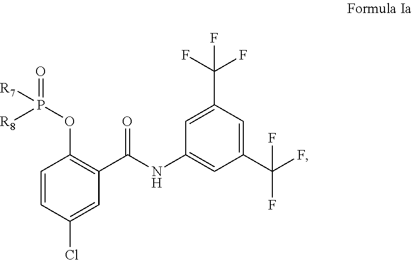

as described in any of Method 3.23-3.31, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the compound of Formula Ia

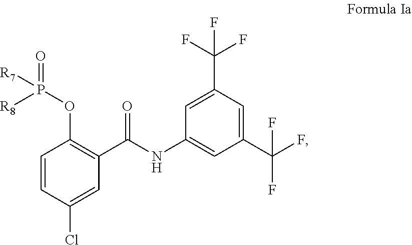

as described in any of Method 3.23-3.31, in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
3.42. Any of Method 3 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising Formula Ia

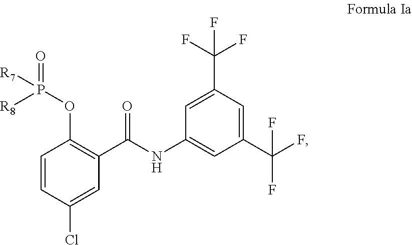

as described in any of Method 3.23-3.31, dissolved therein.
3.43. Any of Method 3.39-3.42 wherein the concentration of

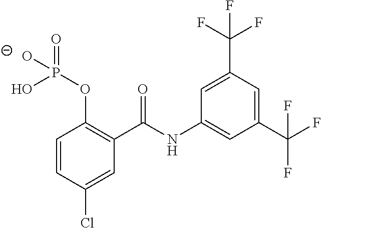 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
3.44. Any of Method 3.39-3.42 wherein the concentration of

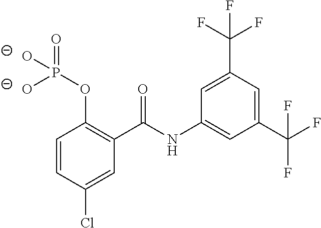 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
3.45. Any of Method 3 et seq. comprising administering a dose of 0.01 or 0.1 or 0.5 mg/kg to 1 or 5 or 10 or 15 mg/kg of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
3.46. Any of Method 3 et seq. comprising administering the pharmaceutically acceptable salt, prodrug, or pharmaceutically acceptable salt prodrug of Formula I, e.g., of N-[3,5-bis(trifluoromethyl)phenyl]-5-chloro-2-hydroxybenzamide, in an amount sufficient to provide a dose of 0.01 or 0.1 or 0.5 mg/kg to 1 or 5 or 10 or 15 mg/kg of the compound of Formula I, e.g., N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
3.47. Any of Method 3 et seq. comprising administering a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide or a pharmaceutically acceptable salt, prodrug (e.g., 2-((3,5-bis(trifluoromethyl)phenyl)carbamoyl)-4-chlorophenyl dihydrogen phosphate), or pharmaceutically acceptable salt prodrug thereof (e.g., Formula Ia as described in any of Method 3.23-3.31), e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
3.48. Any of Method 3 et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 3.23-3.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
3.49. Any of Method 3 et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 3.23-3.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
3.50. Any of Method 3 et seq. comprising administering

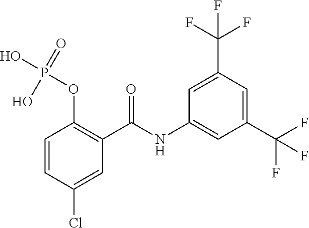 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
3.51. Any of Method 3 et seq. comprising administering

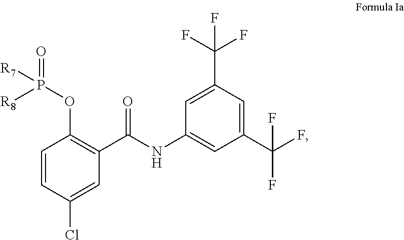

as described in any of Method 3.23-3.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
3.52. Any of Method 3 et seq. wherein the rejection or edema, e.g., transplant rejection, e.g., edema, is consequent to an autograft.
3.53. Any of Method 3 or 3.1-3.51 wherein the rejection or edema, e.g., transplant rejection, e.g., edema, is consequent to a syngeneic graft.
3.54. Any of Method 3 or 3.1-3.51 wherein the rejection or edema, e.g., transplant rejection, e.g., edema, is consequent to an isograft.
3.55. Any of Method 3 or 3.1-3.51 wherein the rejection or edema, e.g., transplant rejection, e.g., edema, is consequent to an allograft.
3.56. Any of Method 3 or 3.1-3.51 wherein the rejection or edema, e.g., transplant rejection, e.g., edema, is consequent to a xenograft.
3.57. Any of Method 3 et seq. wherein the rejection or edema, e.g., transplant rejection, e.g., edema, is consequent to a cell transplant, e.g., hematopoietic stem cell transplant, lymphocyte transplant, or pancreatic islet cell transplant, e.g., hematopoietic stem cell transplant, e.g., lymphocyte transplant, e.g., pancreatic islet cell transplant.
3.58. Any of Method 3 or 3.1-3.56 wherein the rejection or edema, e.g., transplant rejection, e.g., edema, is consequent to a tissue transplant.
3.59. Method 3.58 wherein the tissue is bone, tendon, cartilage, connective tissue, skin, cornea, sclera, heart valve, nerve, or vessel.
3.60. Any of Method 3 or 3.1-3.51 wherein the rejection or edema, e.g., transplant rejection, e.g., edema, is consequent to transplant of an organ or a portion thereof.
3.61. Method 3.60 wherein the organ is a kidney.
3.62. Method 3.60 wherein the organ is the liver.
3.63. Method 3.60 wherein the organ is the pancreas.
3.64. Method 3.60 wherein the organ is a lung.
3.65. Method 3.60 wherein the organ is the heart.
3.66. Method 3.60 wherein the organ is the thymus.
3.67. Method 3.60 wherein the organ is the intestine.
3.68. Method 3.60 wherein the organ is the uterus.
3.69. Any of Method 3 or 3.1-3.51 wherein the rejection or edema, e.g., transplant rejection, e.g., edema, is consequent to a face, limb (e.g., hand), eye, trachea, muscle, or esophagus transplant.
3.70. Any of Method 3 et seq. wherein the rejection or edema, e.g., transplant rejection, e.g., edema, is hyperacute or accelerated rejection, e.g., hyperacute rejection, e.g., accelerated rejection.
3.71. Any of Method 3 or 3.1-3.69 wherein the transplant rejection is acute rejection.
3.72. Any of Method 3 or 3.1-3.69 wherein the transplant rejection is chronic rejection.
3.73. Any of Method 3 et seq. wherein the aquaporin is AQP4.
3.74. Any of Method 3 et seq. wherein the aquaporin is AQP2.
3.75. Any of Method 3 et seq. wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered orally, e.g., tablet, capsule, solution, suspension, or the like.
3.76. Method 3.75 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 3.20, 3.21, or 3.23-3.34, is administered orally.
3.77. Any of Method 3 or 3.1-3.74 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered parenterally.
3.78. Method 3.77 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 3.20, 3.21, or 3.23-3.34, is administered parenterally.
3.79. Method 3.77 or 3.78 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
3.80. Any of Method 3.77-3.79 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 3.20, 3.21, or 3.23-3.34, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
3.81. Any of Method 3.77-3.80 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intravenously, e.g., IV bolus and/or IV infusion, e.g., IV bolus followed by IV infusion.
3.82. Any of Method 3.77-3.81 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 3.20, 3.21, or 3.23-3.34, is administered intravenously, e.g., IV bolus and/or IV infusion, e.g., IV bolus followed by IV infusion.
3.83. Any of Method 3.77-3.80 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intramuscularly, e.g., IM bolus and/or IM infusion, e.g., IM bolus followed by IM infusion.
3.84. Any of Method 3.77-3.81 or 3.83 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 3.20, 3.21, or 3.23-3.34, is administered intramuscularly, e.g., IM bolus and/or IM infusion, e.g., IM bolus followed by IM infusion.
3.85. Any of Method 3.77-3.84 wherein the infusion, e.g., IV or IM, is administered over 10 or 30 minutes to 72 hours, e.g., 30 minutes to 24 hours, e.g., 30 minutes to 12 hours, e.g., 30 minutes to 8 hours, e.g., 30 minutes to 6 hours, e.g., 30 minutes to 4 hours, e.g., 30 minutes to 2 hours, e.g., 30 minutes to 1 hour.
3.86. Any of Method 3 et seq. comprising concurrently or sequentially administering another treatment for transplant rejection.
3.87. Any of Method 3 et seq. comprising concurrently or sequentially administering an immunosuppressant (e.g., a corticosteroid (e.g., prednisone, prednisolone, methylprednisolone, hydrocortisone, dexamethasone), a calcineurin inhibitor (e.g., cyclosporine, tacrolimus), a purine metabolism inhibitor (e.g., azathioprine, mycophenolate mofetil), a rapamycin (e.g., sirolimus, everolimus), an immunosuppressive Ig (e.g., antilymphocyte globulin, antithymocyte globulin, anti-Tac antibody), a monoclonal antibody (mAb) (e.g., OKT3, an anti-IL-2 receptor monoclonal antibody (e.g., basiliximab, daclizumab)), or an agent that inhibits T-cell costimulatory pathways (e.g., a cytotoxic T lymphocyte-associated antigen 4 (CTLA-4)-IgG1 fusion protein, belatacept), or a combination thereof.
3.88. Any of Method 3 et seq. further comprising induction of chimerism using nonmyeloablative pretransplantation treatment (e.g., with cyclophosphamide, thymic irradiation, antithymocte globulin, or cyclosporin, or a combination thereof).
3.89. Any of Method 3 et seq. further comprising total body irradiation.
3.90. Any of Method 3 et seq. wherein the patient is human.
3.91. Any of Method 3 et seq. wherein the onset of action of any of the compounds identified in any of Methods 3, 3.6-3.20, or 3.22-3.31, is fairly rapid.
3.92. Any of Method 3 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 3.20, 3.21, or 3.23-3.34, prior to transplantation, e.g., 12 hours or less, e.g. 8 hours or less, e.g., 6 hours or less, e.g., 3 hours or less, e.g., 2 hours or less, e.g., 1 hour or less, e.g. 30 minutes or less, e.g., 10 or 5 minutes or less, prior to transplantation.
3.93. Any of Method 3 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 3.20, 3.21, or 3.23-3.34, contemporaneously with transplantation.
3.94. Any of Method 3 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 3.20, 3.21, or 3.23-3.34, after transplantation.
3.95. Method 3.94 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 3.20, 3.21, or 3.23-3.34, e.g., comprising administering a pharmaceutically acceptable solution prepared by dissolving 2-((3,5-bis(trifluoromethyl)phenyl)carbamoyl)-4-chlorophenyl dihydrogen phosphate and tris(hydroxymethyl)aminomethane, is administered for 6 months or less after the transplant, e.g., 5 months or less, e.g., 4 months or less, e.g., 3 months or less, e.g., 2 months or less, e.g., 1 month or less, e.g., 3 weeks or less, e.g., 2 weeks or less, e.g., 1 week or less.
3.96. Any of Method 3 et seq. wherein the patient is a transplant donor.
3.97. Any of Method 3 et seq. wherein the patient is a transplant recipient.
3.98. Any of Method 3 et seq. wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 3.20, 3.21, or 3.23-3.34, e.g., is administered before and/or after the transplant.

In another embodiment, provided is a method (Method 4) to inhibit an aquaporin in a patient suffering from transplant rejection, to inhibit an aquaporin to inhibit rejection of transplanted biological material, or to inhibit an aquaporin for prophylaxis, treatment, or control of edema consequent to a transplant, comprising administering to a patient in need thereof an aquaporin inhibitor, e.g., a compound binding to an aquaporin, e.g. AQP4, e.g., an inhibitor of AQP2 or AQP4, e.g., AQP4, in an amount effective to inhibit the aquaporin, e.g., AQP4, wherein the aquaporin inhibitor is a phenylbenzamide, e.g., a compound of Formula I:

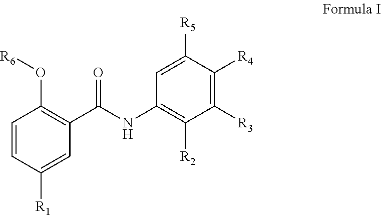

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
4.1. Method 4 comprising inhibiting the aquaporin in the patient suffering from transplant rejection.
4.2. Method 4 comprising inhibiting the aquaporin to inhibit rejection of transplanted biological material.
4.3. Method 4 comprising inhibiting the aquaporin for prophylaxis, treatment, or control edema consequent to a transplant.
4.4. Any of Method 4 et seq. wherein the phenylbenzamide is as described in U.S. Patent Publication No. 2010/0274051.
4.5. Any of Method 4 et seq. wherein the phenylbenzamide is as described in U.S. Pat. Nos. 7,626,042 or 7,700,655.
4.6. Any of Method 4 or 4.1-4.3 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
4.7. Method 4.6 wherein R₁ is selected from chloro and bromo; R₃ and R₅ are both trifluoromethyl; and R₂, R₄ and R₆ are all H, e.g., wherein the compound of Formula I is selected from:

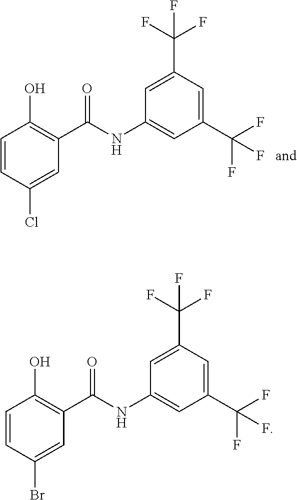 [SMILES: O=C(Nc1cc(C(F)(F)F)cc(C(F)(F)F)c1)c1cc(Cl)ccc1O ; "and" ; O=C(Nc1cc(C(F)(F)F)cc(C(F)(F)F)c1)c1cc(Br)ccc1O]

4.8. Any of Method 4, 4.1-4.3, or 4.6 wherein R₆ is H or acetyl, e.g., H, e.g., acetyl.
4.9. Any of Method 4, 4.1-4.3, or 4.6 wherein R₁ is selected from chloro and bromo; R₃ and R₅ are both trifluoromethyl; and R₂ and R₄ are H and R₆ is acetyl, e.g., wherein the compound of Formula I is selected from:

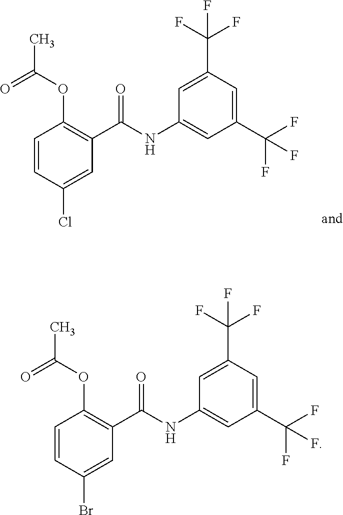 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

4.10. Method 4.7 wherein the compound of Formula I is:

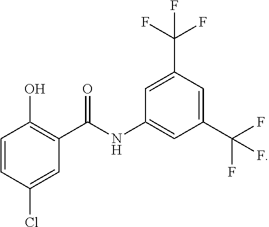 [SMILES: O=C(Nc1cc(C(F)(F)F)cc(C(F)(F)F)c1)c1cc(Cl)ccc1O]

4.11. Any of Method 4, 4.1-4.3, or 4.6 wherein R₁, R₃ and R₅ are each chloro, and R₂, R₄ and R₆ are each H.
4.12. Any of Method 4, 4.1-4.3, or 4.6 wherein R₁, R₃ and R₅ are each trifluoromethyl, and R₂, R₄ and R₆ are each H.
4.13. Any of Method 4, 4.1-4.3, or 4.6 wherein R₆ is C₁₋₄ acyl (e.g. acetyl).
4.14. Any of Method 4, 4.1-4.3, or 4.6 wherein R₆ is the residue of an amino acid.
4.15. Any of Method 4, 4.1-4.3, or 4.6 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
4.16. Any of Method 4, 4.1-4.3, or 4.6 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
4.17. Any of Method 4, 4.1-4.3, or 4.6 wherein R₆ is a (morpholin-4-yl)carbonyl group.
4.18. Any of Method 4, 4.1-4.3, or 4.6 wherein R₆ is a phosphono (—PO₃), which may be substituted, e.g. dibenzylphosphono (—P(═O)(OCH₂C₆H₅)₂), or unsubstituted (—P(═O)(OH)₂).
4.19. Method 4.18 wherein R₆ is ,,P(═O)(OH)₂.
4.20. Method 4.19 wherein the prodrug of Formula I is 2-((3,5-bis(trifluoromethyl)phenyl)carbamoyl)-4-chlorophenyl dihydrogen phosphate:

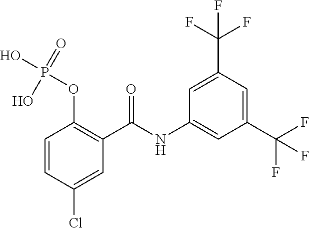 [SMILES: O=C(Nc1cc(C(F)(F)F)cc(C(F)(F)F)c1)c1cc(Cl)ccc1OP(=O)(O)O]

or a pharmaceutically acceptable salt thereof.
4.21. Method 4.20 comprising administering a pharmaceutically acceptable solution comprising a pharmaceutically acceptable salt of 2-((3,5-bis(trifluoromethyl)phenyl)carbamoyl)-4-chlorophenyl dihydrogen phosphate dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
4.22. Method 4.18 wherein the prodrug of Formula I is:

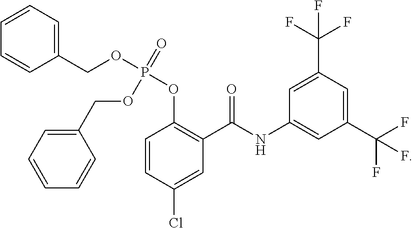 [SMILES: O=C(Nc1cc(C(F)(F)F)cc(C(F)(F)F)c1)c1cc(Cl)ccc1OP(=O)(OCc1ccccc1)OCc1ccccc1]

4.23. Method 4.18 wherein the pharmaceutically acceptable salt prodrug of Formula I is a compound of Formula Ia:

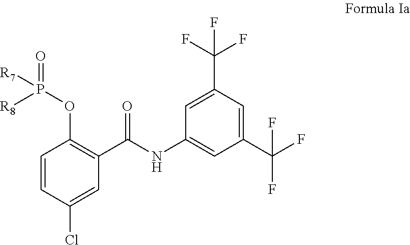

wherein one of R₇ and R₈ is OH and the other is O⁻Q⁺ or both R₇ and R₈ are O⁻Q⁺ wherein each Q⁺ is independently a pharmaceutically acceptable cation.
4.24. Method 4.23 wherein one of R₇ and R₈ is OH and the other is O⁻Q⁺.
4.25. Method 4.23 wherein both R₇ and R₈ are O⁻Q⁺.
4.26. Any of Method 4.23-4.25 wherein each Q⁺ is independently Na⁺ or K⁺.
4.27. Method 4.26 wherein each Q⁺ is Na⁺.
4.28. Method 4.27 wherein the pharmaceutically acceptable salt prodrug of Formula Ia is:

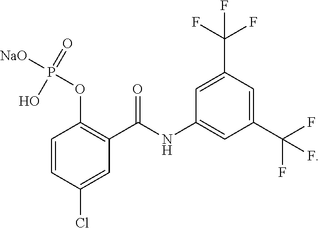

4.29. Method 4.27 wherein the pharmaceutically acceptable salt prodrug of Formula Ia is:

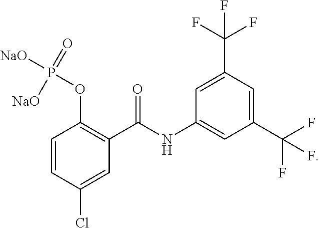

4.30. Any of Method 4.23-4.25 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂⁺, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
4.31. Method 4.30 wherein each Q⁺ is protonated tris(hydroxymethyl)aminomethane.
4.32. Any of Method 4.23-4.31 comprising administering a pharmaceutically acceptable solution comprising Formula Ia dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
4.33. Method 4.21 or 4.32 wherein the concentration of

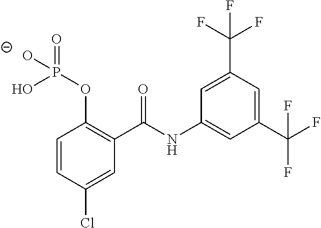 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
4.34. Method 4.21 or 4.32 wherein the concentration of

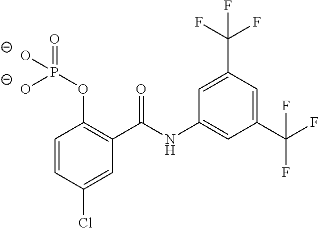 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
4.35. Any of Method 4 or 4.1-4.3 wherein the compound of Formula I is:

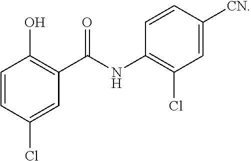 [SMILES: N#Cc1ccc(NC(=O)c2cc(Cl)ccc2O)c(Cl)c1]

4.36. Any of Method 4 or 4.1-4.3 wherein the phenylbenzamide is:

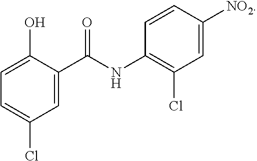 [SMILES: O=C(Nc1ccc([N+](=O)[O-])cc1Cl)c1cc(Cl)ccc1O]

4.37. Any of Method 4 et seq. comprising administering 0.1 or 0.25 mg to 2.0 g of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug thereof in an amount sufficient to provide 0.1 or 0.25 mg to 2.0 g of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
4.38. Any of Method 4 et seq. comprising administering 0.1 or 0.25 mg to 2.0 g of N-[3,5-bis(trifluoromethyl)phenyl]-5-chloro-2-hydroxybenzamide or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
4.39. Any of Method 4 et seq. comprising administering 0.1 or 0.25 mg to 2.0 g of

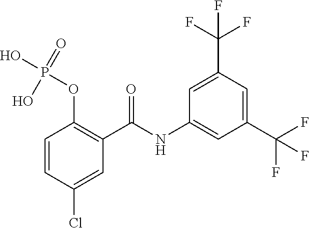 [SMILES: O=C(Nc1cc(C(F)(F)F)cc(C(F)(F)F)c1)c1cc(Cl)ccc1OP(=O)(O)O]

or a pharmaceutically acceptable salt thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering

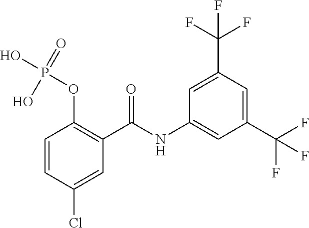 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
4.40. Any of Method 4 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising

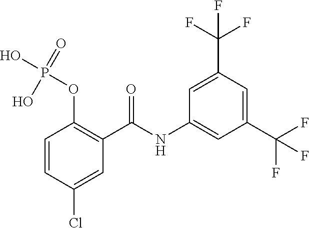 [SMILES: O=C(Nc1cc(C(F)(F)F)cc(C(F)(F)F)c1)c1cc(Cl)ccc1OP(=O)(O)O]

or a pharmaceutically acceptable salt thereof dissolved therein.
4.41. Any of Method 4 et seq. comprising administering 0.1 or 0.25 mg to 2.0 g of the compound of Formula Ia

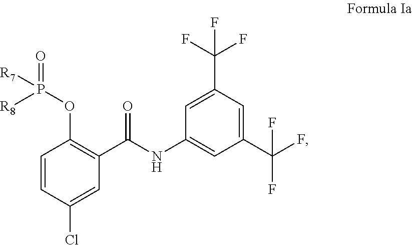

as described in any of Method 4.23-4.31, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the compound of Formula Ia

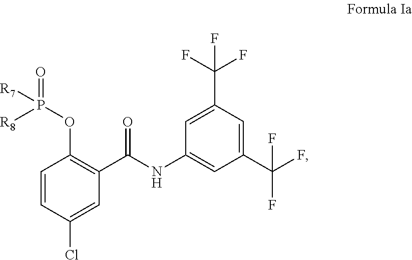

as described in any of Method 4.23-4.31, in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
4.42. Any of Method 4 et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising Formula Ia

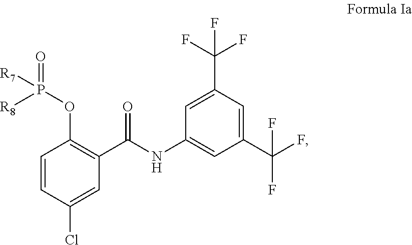

as described in any of Method 4.23-4.31, dissolved therein.
4.43. Any of Method 4.39-4.42 wherein the concentration of

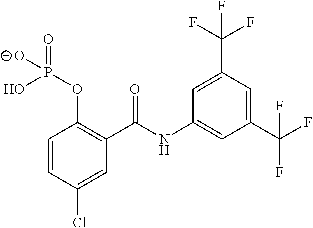 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
4.44. Any of Method 4.39-4.42 wherein the concentration of

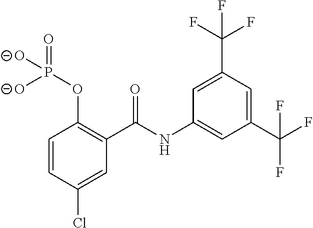 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
4.45. Any of Method 4 et seq. comprising administering a dose of 0.01 or 0.1 or 0.5 mg/kg to 1 or 5 or 10 or 15 mg/kg of the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
4.46. Any of Method 4 et seq. comprising administering the pharmaceutically acceptable salt, prodrug, or pharmaceutically acceptable salt prodrug of Formula I, e.g., of N-[3,5-bis(trifluoromethyl)phenyl]-5-chloro-2-hydroxybenzamide, in an amount sufficient to provide a dose of 0.01 or 0.1 or 0.5 mg/kg to 1 or 5 or 10 or 15 mg/kg of the compound of Formula I, e.g., N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
4.47. Any of Method 4 et seq. comprising administering a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide or a pharmaceutically acceptable salt, prodrug (e.g., 2-((3,5-bis(trifluoromethyl)phenyl)carbamoyl)-4-chlorophenyl dihydrogen phosphate), or pharmaceutically acceptable salt prodrug thereof (e.g., Formula Ia as described in any of Method 4.23-4.31), e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
4.48. Any of Method 4 et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 4.23-4.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
4.49. Any of Method 4 et seq. comprising administering the prodrug or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 4.23-4.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
4.50. Any of Method 4 et seq. comprising administering

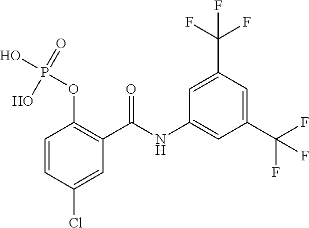 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
4.51. Any of Method 4 et seq. comprising administering

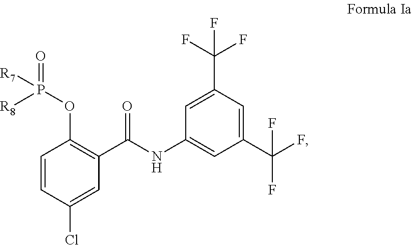

as described in any of Method 4.23-4.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
4.52. Any of Method 4 et seq. wherein the rejection or edema, e.g., transplant rejection, e.g., edema, is consequent to an autograft.
4.53. Any of Method 4 or 4.1-4.51 wherein the rejection or edema, e.g., transplant rejection, e.g., edema, is consequent to a syngeneic graft.
4.54. Any of Method 4 or 4.1-4.51 wherein the rejection or edema, e.g., transplant rejection, e.g., edema, is consequent to an isograft.
4.55. Any of Method 4 or 4.1-4.51 wherein the rejection or edema, e.g., transplant rejection, e.g., edema, is consequent to an allograft.
4.56. Any of Method 4 or 4.1-4.51 wherein the rejection or edema, e.g., transplant rejection, e.g., edema, is consequent to a xenograft.
4.57. Any of Method 4 et seq. wherein the rejection or edema, e.g., transplant rejection, e.g., edema, is consequent to a cell transplant, e.g., hematopoietic stem cell transplant, lymphocyte transplant, or pancreatic islet cell transplant, e.g., hematopoietic stem cell transplant, e.g., lymphocyte transplant, e.g., pancreatic islet cell transplant.
4.58. Any of Method 4 or 4.1-4.56 wherein the rejection or edema, e.g., transplant rejection, e.g., edema, is consequent to a tissue transplant.
4.59. Method 4.58 wherein the tissue is bone, tendon, cartilage, connective tissue, skin, cornea, sclera, heart valve, nerve, or vessel.
4.60. Any of Method 4 or 4.1-4.56 wherein the rejection or edema, e.g., transplant rejection, e.g., edema, is consequent to transplant of an organ or a portion thereof.
4.61. Method 4.60 wherein the organ is a kidney.
4.62. Method 4.60 wherein the organ is the liver.
4.63. Method 4.60 wherein the organ is the pancreas.
4.64. Method 4.60 wherein the organ is a lung.
4.65. Method 4.60 wherein the organ is the heart.
4.66. Method 4.60 wherein the organ is the thymus.
4.67. Method 4.60 wherein the organ is the intestine.
4.68. Method 4.60 wherein the organ is the uterus.
4.69. Any of Method 4 or 4.1-4.56 wherein the rejection or edema, e.g., transplant rejection, e.g., edema, is consequent to a face, limb (e.g., hand), eye, trachea, muscle, or esophagus transplant.
4.70. Any of Method 4 et seq. wherein the transplant rejection is hyperacute or accelerated rejection, e.g., hyperacute rejection, e.g., accelerated rejection.
4.71. Any of Method 4 or 4.1-4.69 wherein the transplant rejection is acute rejection.
4.72. Any of Method 4 or 4.1-4.69 wherein the transplant rejection is chronic rejection.
4.73. Any of Method 4 et seq. wherein the aquaporin is AQP4.
4.74. Any of Method 4 et seq. wherein the aquaporin is AQP2.
4.75. Any of Method 4 et seq. wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered orally, e.g., tablet, capsule, solution, suspension, or the like.
4.76. Method 4.75 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 4.20, 4.21, or 4.23-4.34, is administered orally.
4.77. Any of Method 4 or 4.1-4.74 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered parenterally.
4.78. Method 4.77 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 4.20, 4.21, or 4.23-4.34, is administered parenterally.
4.79. Method 4.77 or 4.78 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
4.80. Any of Method 4.77-4.79 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 4.20, 4.21, or 4.23-4.34, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
4.81. Any of Method 4.77-4.80 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intravenously, e.g., IV bolus and/or IV infusion, e.g., IV bolus followed by IV infusion.
4.82. Any of Method 4.77-4.81 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 4.20, 4.21, or 4.23-4.34, is administered intravenously, e.g., IV bolus and/or IV infusion, e.g., IV bolus followed by IV infusion.
4.83. Any of Method 4.77-4.80 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intramuscularly, e.g., IM bolus and/or IM infusion, e.g., IM bolus followed by IM infusion.
4.84. Any of Method 4.77-4.81 or 4.83 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 4.20, 4.21, or 4.23-4.34, is administered intramuscularly, e.g., IM bolus and/or IM infusion, e.g., IM bolus followed by IM infusion.
4.85. Any of Method 4.77-4.84 wherein the infusion, e.g., IV or IM, is administered over 10 or 30 minutes to 72 hours, e.g., 30 minutes to 24 hours, e.g., 30 minutes to 12 hours, e.g., 30 minutes to 8 hours, e.g., 30 minutes to 6 hours, e.g., 30 minutes to 4 hours, e.g., 30 minutes to 2 hours, e.g., 30 minutes to 1 hour.
4.86. Any of Method 4 et seq. comprising concurrently or sequentially administering another treatment for transplant rejection.
4.87. Any of Method 4 et seq. comprising concurrently or sequentially administering an immunosuppressant (e.g., a corticosteroid (e.g., prednisone, prednisolone, methylprednisolone, hydrocortisone, dexamethasone), a calcineurin inhibitor (e.g., cyclosporine, tacrolimus), a purine metabolism inhibitor (e.g., azathioprine, mycophenolate mofetil), a rapamycin (e.g., sirolimus, everolimus), an immunosuppressive Ig (e.g., antilymphocyte globulin, antithymocyte globulin, anti-Tac antibody), a monoclonal antibody (mAb) (e.g., OKT3, an anti-IL-2 receptor monoclonal antibody (e.g., basiliximab, daclizumab)), or an agent that inhibits T-cell costimulatory pathways (e.g., a cytotoxic T lymphocyte-associated antigen 4 (CTLA-4)-IgG1 fusion protein, belatacept), or a combination thereof.
4.88. Any of Method 4 et seq. further comprising induction of chimerism using nonmyeloablative pretransplantation treatment (e.g., with cyclophosphamide, thymic irradiation, antithymocte globulin, or cyclosporin, or a combination thereof).
4.89. Any of Method 4 et seq. further comprising total body irradiation.
4.90. Any of Method 4 et seq. wherein the patient is human.
4.91. Any of Method 4 et seq. wherein the onset of action of any of the compounds identified in any of Methods 4, 4.6-4.20, or 4.22-4.31, is fairly rapid.
4.92. Any of Method 4 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 4.20, 4.21, or 4.23-4.34, prior to transplantation, e.g., 12 hours or less, e.g. 8 hours or less, e.g., 6 hours or less, e.g., 3 hours or less, e.g., 2 hours or less, e.g., 1 hour or less, 30 minutes or less, e.g., 10 or 5 minutes or less, prior to transplantation.
4.93. Any of Method 4 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 4.20, 4.21, or 4.23-4.34, contemporaneously with transplantation.
4.94. Any of Method 4 et seq. comprising administering the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 4.20, 4.21, or 4.23-4.34, after transplantation.
4.95. Method 4.94 wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 4.20, 4.21, or 4.23-4.34, e.g., comprising administering a pharmaceutically acceptable solution prepared by dissolving 2-((3,5-bis(trifluoromethyl)phenyl)carbamoyl)-4-chlorophenyl dihydrogen phosphate and tris(hydroxymethyl)aminomethane, is administered for 6 months or less after the transplant, e.g., 5 months or less, e.g., 4 months or less, e.g., 3 months or less, e.g., 2 months or less, e.g., 1 month or less, e.g., 3 weeks or less, e.g., 2 weeks or less, e.g., 1 week or less.
4.96. Any of Method 2 or 3 or 2.1-2.95 wherein the patient is a transplant donor.
4.97. Any of Method 2 or 3 or 2.1-2.95 wherein the patient is a transplant recipient.
4.98. Any of Method 4 et seq. wherein the aquaporin inhibitor, e.g., the compound binding to the aquaporin, e.g., the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 4.20, 4.21, or 4.23-4.34, e.g., is administered before and/or after the transplant.

Further provided is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, for use in the treatment or prophylaxis of transplant rejection, inhibiting rejection of transplanted biological material, or prophylaxis, treatment, or control of edema consequent to a transplant, e.g., for use in any of Methods 1, 1.1, et seq.

Further provided is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in the manufacture of a medicament for the treatment or prophylaxis of transplant rejection, inhibiting rejection of transplanted biological material, or prophylaxis, treatment, or control of edema consequent to a transplant, e.g., for use in any of Methods 1, 1.1, et seq.

Further provided is a pharmaceutical composition comprising a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in combination with a pharmaceutically acceptable diluent or carrier for use in the treatment or prophylaxis of transplant rejection, inhibiting rejection of transplanted biological material, or prophylaxis, treatment, or control of edema consequent to a transplant, e.g., for use in any of Methods 1, 1.1, et seq.

Further provided is an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, for use in the treatment or prophylaxis of transplant rejection, inhibiting rejection of transplanted biological material, or prophylaxis, treatment, or control of edema consequent to a transplant, e.g., for use in any of Methods 2, 2.1, et seq.

Further provided is an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in the manufacture of a medicament for the treatment or prophylaxis of transplant rejection, inhibiting rejection of transplanted biological material, or prophylaxis, treatment, or control of edema consequent to a transplant, e.g., for use in any of Methods 2, 2.1, et seq.

Further provided is a pharmaceutical composition comprising an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in combination with a pharmaceutically acceptable diluent or carrier for use in the treatment or prophylaxis of transplant rejection, inhibiting rejection of transplanted biological material, or prophylaxis, treatment, or control of edema consequent to a transplant, e.g., for use in any of Methods 2, 2.1, et seq.

Further provided is use of a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in an amount effective to inhibit an aquaporin for the treatment or prophylaxis of transplant rejection, for inhibiting rejection of transplanted biological material, or for the prophylaxis, treatment, or control of edema consequent to a transplant, e.g., for use in any of Methods 3, 3.1, et seq.

Further provided is use of a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in an amount effective to inhibit an aquaporin in the manufacture of a medicament for the treatment or prophylaxis of transplant rejection, inhibiting rejection of transplanted biological material, or prophylaxis, treatment, or control of edema consequent to a transplant, e.g., for use in any of Methods 3, 3.1, et seq.

Further provided is a pharmaceutical composition comprising a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in an amount effective to inhibit an aquaporin in combination with a pharmaceutically acceptable diluent or carrier for use in the treatment or prophylaxis of transplant rejection, inhibiting rejection of transplanted biological material, or prophylaxis, treatment, or control of edema consequent to a transplant, e.g., for use in any of Methods 3, 3.1, et seq.

Further provided are methods of treating a cell, tissue, or organ donor with a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate as hereinbefore described, using the methods as hereinbefore described, e.g., Methods 1, 1.1, et seq., Methods 2, 2.1, et seq., Methods 3, 3.1, et seq., Methods 4, 4.1, et seq. For example, provided is a method (Method A) for treatment of a cell, tissue, or organ donor comprising administering to the donor, before and/or after removal of the cell, tissue, or organ, an effective amount of a phenylbenzamide, e.g., an effective amount of a compound of Formula I:

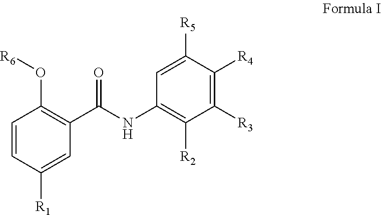

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
A.1. Method A et seq. wherein the phenylbenzamide is as described in U.S. Patent Publication No. 2010/0274051.
A.2. Method A or A.1 wherein the phenylbenzamide is as described in U.S. Pat. Nos. 7,626,042 or 7,700,655.
A.3. Method A wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
A.4. Method A.3 wherein R₁ is selected from chloro and bromo; R₃ and R₅ are both trifluoromethyl; and R₂, R₄ and R₆ are all H, e.g., wherein the compound of Formula I is selected from:

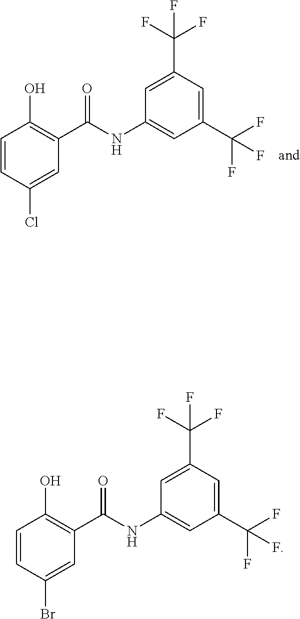 [SMILES: O=C(Nc1cc(C(F)(F)F)cc(C(F)(F)F)c1)c1cc(Cl)ccc1O ; O=C(Nc1cc(C(F)(F)F)cc(C(F)(F)F)c1)c1cc(Br)ccc1O]

A.5. Method A or A.3 wherein R₆ is H or acetyl, e.g., H, e.g., acetyl.
A.6. Method A or A.3 wherein R₁ is selected from chloro and bromo; R₃ and R₅ are both trifluoromethyl; and R₂ and R₄ are H and R₆ is acetyl, e.g., wherein the compound of Formula I is selected from:

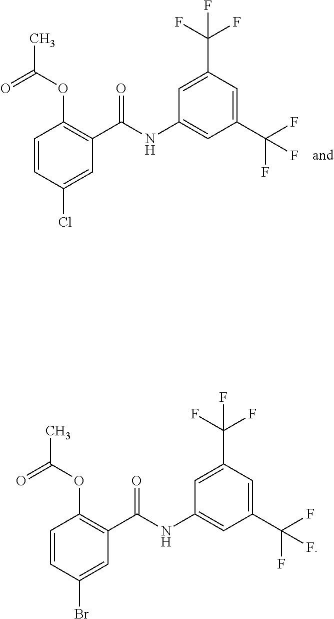 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

A.7. Method A.4 wherein the compound of Formula I is:

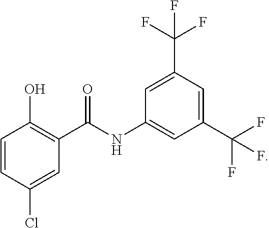 [SMILES: O=C(Nc1cc(C(F)(F)F)cc(C(F)(F)F)c1)c1cc(Cl)ccc1O]

A.8. Method A.3 wherein R₁, R₃ and R₅ are each chloro, and R₂, R₄ and R₆ are each H.
A.9. Method A.3 wherein R₁, R₃ and R₅ are each trifluoromethyl, and R₂, R₄ and R₆ are each H.
A.10. Method A or A.3 wherein R₆ is C₁₋₄ acyl (e.g. acetyl).
A.11. Method A or A.3 wherein R₆ is the residue of an amino acid.
A.12. Method A or A.3 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
A.13. Method A or A.3 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
A.14. Method A or A.3 wherein R₆ is a (morpholin-4-yl)carbonyl group.
A.15. Method A or A.3 wherein R₆ is a phosphono (—PO₃), which may be substituted, e.g. dibenzylphosphono (—P(═O)(OCH₂C₆H₅)₂), or unsubstituted (—P(═O)(OH)₂).
A.16. Method A.15 wherein R₆ is ,,P(═O)(OH)₂.
A.17. Method A.16 wherein the prodrug of Formula I is 2-((3,5-bis(trifluoromethyl)phenyl)carbamoyl)-4-chlorophenyl dihydrogen phosphate:

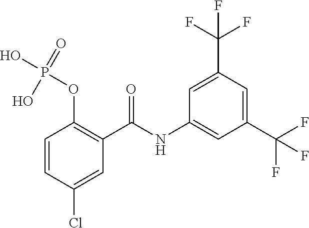 [SMILES: O=C(Nc1cc(C(F)(F)F)cc(C(F)(F)F)c1)c1cc(Cl)ccc1OP(=O)(O)O]

or a pharmaceutically acceptable salt thereof.
A.18. Method A. 17 comprising administering a pharmaceutically acceptable solution comprising a pharmaceutically acceptable salt of 2-((3,5-bis(trifluoromethyl)phenyl)carbamoyl)-4-chlorophenyl dihydrogen phosphate dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
A.19. Method A.15 wherein the prodrug of Formula I is:

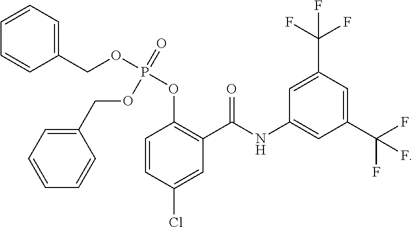 [SMILES: O=C(Nc1cc(C(F)(F)F)cc(C(F)(F)F)c1)c1cc(Cl)ccc1OP(=O)(OCc1ccccc1)OCc1ccccc1]

A.20. Method A. 15 wherein the pharmaceutically acceptable salt prodrug of Formula I is a compound of Formula Ia:

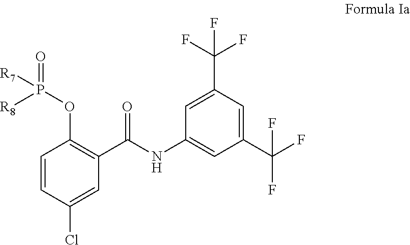

wherein one of R₇ and R₈ is OH and the other is O⁻Q⁺ or both R₇ and R₈ are O⁻Q⁺ wherein each Q⁺ is independently a pharmaceutically acceptable cation.
A.21. Method A.20 wherein one of R₇ and R₈ is OH and the other is O⁻Q⁺.
A.22. Method A.20 wherein both R₇ and R₈ are O⁻Q⁺.
A.23. Any of Method A.20-A.22 wherein each Q⁺ is independently N^{a} or K⁺.
A.24. Method A.23 wherein each Q⁺ is N⁺.
A.25. Method A.24 wherein the pharmaceutically acceptable salt prodrug of Formula Ia is:

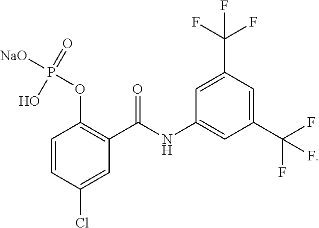

A.26. Method A.24 wherein the pharmaceutically acceptable salt prodrug of Formula Ia is:

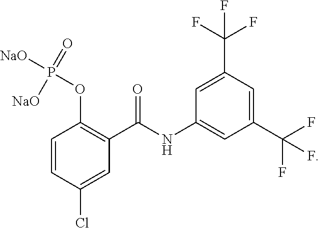

A.27. Any of Method A.20-A.22 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂⁺, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
A.28. Method A.27 wherein each Q⁺ is protonated tris(hydroxymethyl)aminomethane.
A.29. Any of Method A.20-A.28 comprising administering a pharmaceutically acceptable solution comprising Formula Ia dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
A.30. Method A. 18 or A.29 wherein the concentration of

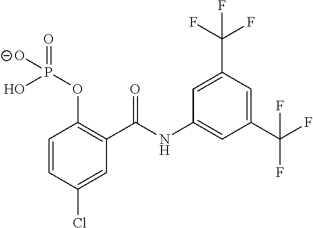 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
A.31. Method A. 18 or A.29 wherein the concentration of

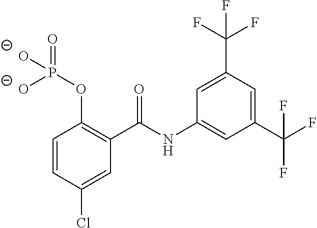 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
A.32. Method A wherein the compound of Formula I is:

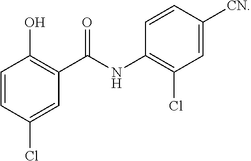 [SMILES: N#Cc1ccc(NC(=O)c2cc(Cl)ccc2O)c(Cl)c1]

A.33. Method A wherein the phenylbenzamide is:

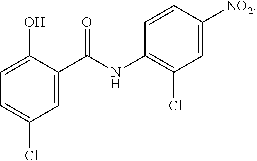 [SMILES: O=C(Nc1ccc([N+](=O)[O-])cc1Cl)c1cc(Cl)ccc1O]

A.34. Any of Method A et seq. comprising administering 0.1 or 0.25 mg to 2.0 g of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug thereof in an amount sufficient to provide 0.1 or 0.25 mg to 2.0 g of the phenylbenzamide, e.g., the compound of Formula I, e.g., N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
A.35. Any of Method A et seq. comprising administering 0.1 or 0.25 mg to 2.0 g of N-[3,5-bis(trifluoromethyl)phenyl]-5-chloro-2-hydroxybenzamide or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the pharmaceutically acceptable salt, prodrug, or pharmaceutically acceptable salt prodrug in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
A.36. Any of Method A et seq. comprising administering 0.1 or 0.25 mg to 2.0 g of

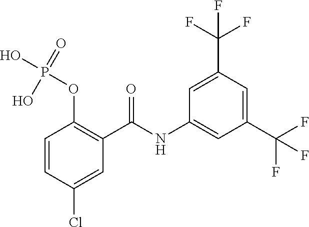 [SMILES: O=C(Nc1cc(C(F)(F)F)cc(C(F)(F)F)c1)c1cc(Cl)ccc1OP(=O)(O)O]

or a pharmaceutically acceptable salt thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering

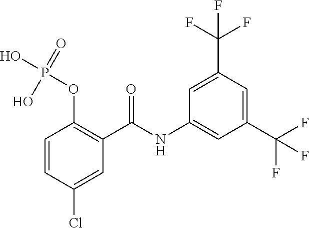 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
A.37. Any of Method A et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising

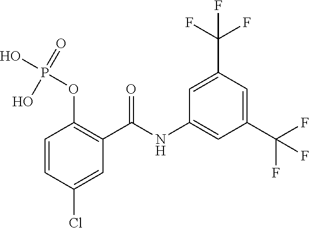 [SMILES: O=C(Nc1cc(C(F)(F)F)cc(C(F)(F)F)c1)c1cc(Cl)ccc1OP(=O)(O)O]

or a pharmaceutically acceptable salt thereof dissolved therein.
A.38. Any of Method A et seq. comprising administering 0.1 or 0.25 mg to 2.0 g of the compound of Formula Ia

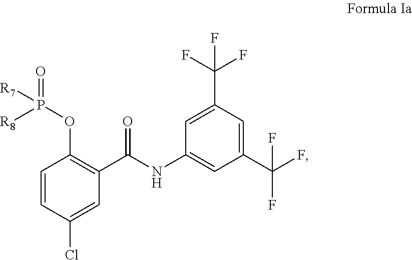

as described in any of Method A.20-A.28, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering the compound of Formula Ia

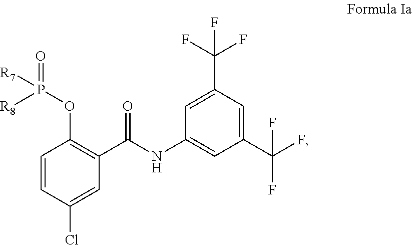

as described in any of Method A.20-A.28, in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
A.39. Any of Method A et seq. comprising administering a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising Formula Ia

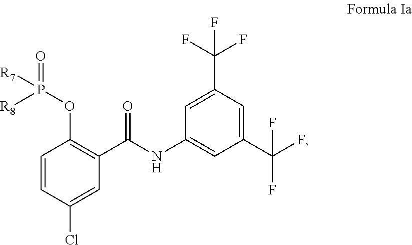

as described in any of Method A.20-A.28, dissolved therein.
A.40. Any of Method A.36-A.39 wherein the concentration of

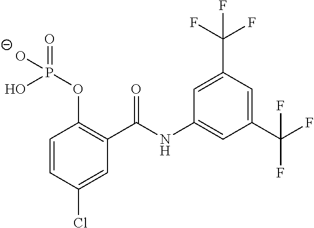 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
A.41. Any of Method A.36-A.39 wherein the concentration of

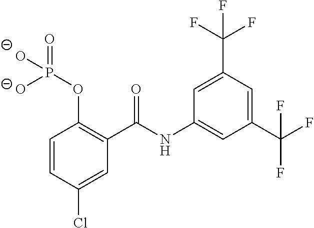 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
A.42. Any of Method A et seq. comprising administering a dose of 0.01 or 0.1 or 0.5 mg/kg to 1 or 5 or 10 or 15 mg/kg of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
A.43. Any of Method A et seq. comprising administering the pharmaceutically acceptable salt, prodrug, or pharmaceutically acceptable salt prodrug of Formula I, e.g., of N-[3,5-bis(trifluoromethyl)phenyl]-5-chloro-2-hydroxybenzamide, in an amount sufficient to provide a dose of 0.01 or 0.1 or 0.5 mg/kg to 1 or 5 or 10 or 15 mg/kg of the compound of Formula I, e.g., N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
A.44. Any of Method A et seq. comprising administering a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide or a pharmaceutically acceptable salt, prodrug (e.g., 2-((3,5-bis(trifluoromethyl)phenyl)carbamoyl)-4-chlorophenyl dihydrogen phosphate), or pharmaceutically acceptable salt prodrug thereof (e.g., Formula Ia as described in any of Method A.20-A.28), e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
A.45. Any of Method A et seq. comprising administering the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method A.20-A.28, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
A.46. Any of Method A et seq. comprising administering the prodrug or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method
A.20-A.28, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
A.47. Any of Method A et seq. comprising administering

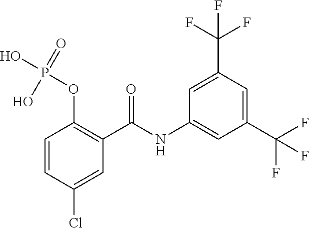 [SMILES: O=C(Nc1cc(C(F)(F)F)cc(C(F)(F)F)c1)c1cc(Cl)ccc1OP(=O)(O)O]

or a pharmaceutically acceptable salt in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
A.48. Any of Method A et seq. comprising administering

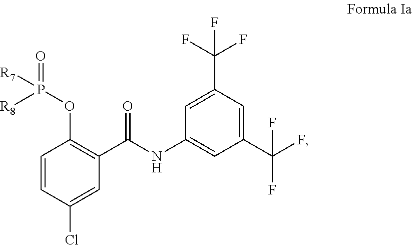

as described in any of Method A.20-A.28, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
A.49. Any of Method A or A.1-A.48 wherein the donor is a tissue donor.
A.50. Method A.49 wherein the tissue is bone, tendon, cartilage, connective tissue, skin, cornea, sclera, heart valve, nerve, and vessel.
A.51. Any of Method A or A.1-A.48 wherein the donor is an organ or portion thereof donor.
A.52. Method A.51 wherein the organ is a kidney.
A.53. Method A.51 wherein the organ is the liver.
A.54. Method A.51 wherein the organ is the pancreas.
A.55. Method A.51 wherein the organ is a lung.
A.56. Method A.51 wherein the organ is the heart.
A.57. Method A.51 wherein the organ is the thymus.
A.58. Method A.51 wherein the organ is the intestine.
A.59. Method A.51 wherein the organ is the uterus.
A.60. Any of Method A or A.1-A.48 wherein the donor is a face, limb (e.g., hand), eye, trachea, muscle, or esophagus donor.
A.61. Any of Method A et seq. wherein the aquaporin is AQP4.
A.62. Any of Method A et seq. wherein the aquaporin is AQP2.
A.63. Any of Method A et seq. wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered orally, e.g., tablet, capsule, solution, suspension, or the like.
A.64. Method A.63 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method A.17, A.18, or A.20-A.31, is administered orally.
A.65. Any of Method A or A.1-1.62 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered parenterally.
A.66. Method A.65 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method A.17, A.18, or A.20-A.31, is administered parenterally.
A.67. Method A.65 or A.66 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
A.68. Any of Methods A.65-1.67 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method A.17, A.18, or A.20-A.31, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
A.69. Any of Method A.65-A.68 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intravenously, e.g., IV bolus and/or IV infusion, e.g., IV bolus followed by IV infusion.
A.70. Any of Method A.65-A.69 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31, is administered intravenously, e.g., IV bolus and/or IV infusion, e.g., IV bolus followed by IV infusion.
A.71. Any of Method A.65-A.68 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intramuscularly, e.g., IM bolus and/or IM infusion, e.g., IM bolus followed by IM infusion.
A.72. Any of Method A.65-A.68 or A.71 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31, is administered intramuscularly, e.g., IM bolus and/or IM infusion, e.g., IM bolus followed by IM infusion.
A.73. Any of Method A.65-A.72 wherein the infusion, e.g., IV or IM, is administered over 10 or 30 minutes to 72 hours, e.g., 30 minutes to 24 hours, e.g., 30 minutes to 12 hours, e.g., 30 minutes to 8 hours, e.g., 30 minutes to 6 hours, e.g., 30 minutes to 4 hours, e.g., 30 minutes to 2 hours, e.g., 30 minutes to 1 hour.
A.74. Any of Method A et seq. comprising concurrently or sequentially administering another treatment for transplant rejection.
A.75. Any of Method A et seq. comprising concurrently or sequentially administering an immunosuppressant (e.g., a corticosteroid (e.g., prednisone, prednisolone, methylprednisolone, hydrocortisone, dexamethasone), a calcineurin inhibitor (e.g., cyclosporine, tacrolimus), a purine metabolism inhibitor (e.g., azathioprine, mycophenolate mofetil), a rapamycin (e.g., sirolimus, everolimus), an immunosuppressive Ig (e.g., antilymphocyte globulin, antithymocyte globulin, anti-Tac antibody), a monoclonal antibody (mAb) (e.g., OKT3, an anti-IL-2 receptor monoclonal antibody (e.g., basiliximab, daclizumab)), or an agent that inhibits T-cell costimulatory pathways (e.g., a cytotoxic T lymphocyte-associated antigen 4 (CTLA-4)-IgG1 fusion protein, belatacept), or a combination thereof.
A.76. Any of Method A et seq. further comprising induction of chimerism using nonmyeloablative pretransplantation treatment (e.g., with cyclophosphamide, thymic irradiation, antithymocte globulin, or cyclosporin, or a combination thereof).
A.77. Any of Method A et seq. further comprising total body irradiation.
A.78. Any of Method A et seq. wherein the patient is human.
A.79. Any of Method A et seq. wherein the onset of action of any of the compounds identified in any of Methods A, A.3-A.17, or A.19-A.28, is fairly rapid.
A.80. Any of Method A et seq. comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31, e.g., comprising administering a pharmaceutically acceptable solution prepared by dissolving 2-((3,5-bis(trifluoromethyl)phenyl)carbamoyl)-4-chlorophenyl dihydrogen phosphate and tris(hydroxymethyl)aminomethane, prior to removal of the cell, tissue, or organ, e.g., 12 hours or less, e.g. 8 hours or less, e.g., 6 hours or less, e.g., 3 hours or less, e.g., 2 hours or less, e.g., 1 hour or less, e.g., 30 minutes or less, e.g., 10 or 5 minutes or less.
A.81. Any of Method A et seq. comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31, contemporaneously with removal of the cell, tissue, or organ.
A.82. Any of Method A et seq. comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31, after removal of the cell, tissue, or organ.
A.83. Method A.82 wherein the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31, is administered for 6 months or less after removal of the cell, tissue, or organ, e.g., 5 months or less, e.g., 4 months or less, e.g., 3 months or less, e.g., 2 months or less, e.g., 1 month or less, e.g., 3 weeks or less, e.g., 2 weeks or less, e.g., 1 week or less.
A.84. Method A.80 wherein the donor is perfused with the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31.
A.85. Method A.80 or A.84 wherein the concentration of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31, is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
A.86. Any of Method A.80, A.84, or A.85 comprising concurrently or sequentially administering a preservation solution, e.g., a preservation solution further comprising the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31.
A.87. Method A.86 wherein the preservation solution comprises an osmotic active agent (e.g., lactogionate, raffinose, citrate, gluconate), an electrolyte (N⁺, K⁺, Ca²⁺, Mg²⁺), an H⁺ ion buffer (phosphate, histidine, N-(2-hydroxyethyl)-piperazine-N′-2-ethanesulfonic acid (HEPES) buffer), a colloid (e.g., albumin, hydroxyethyl starch), a metabolic inhibitor (e.g., allopurinol, antiprotease, chlorpromazine), a metabolite (e.g., adenosine, glutathione), or an antioxidant (e.g., amino steroid, vitamin E, deferoxamine (Desferal), or a combination thereof.
A.88. Method A.86 or A.87 wherein the preservation solution comprises mannitol.
A.89. Any of Method A.86-A.88 wherein the preservation solution is Collins solution, Euro-Collins solution, Ross-Marshall citrate solution, histidine tryptophan ketoglutarate solution, phosphate-buffered sucrose solution, University of Wisconsin solution, Celsior solution, Kyoto ET solution, or IGL-1 solution, e.g., University of Wisconsin solution.
A.90. Any of Method A.86-A.88 wherein the preservation solution is Ringer's solution.
A.91. Any of Method 8 et seq. wherein the cell, tissue, or organ of the organ donor are perfused, e.g., prior and/or contemporaneously with removal of the cell, tissue, or organ, with the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method A.17, A.18, or A.20-A.31.

Further provided is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, for treatment of a cell, tissue, or organ donor, e.g., for use in any of Methods A, A.1, et seq.

Further provided is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in the manufacture of a medicament for treatment of a cell, tissue, or organ donor, e.g., for use in any of Methods A, A.1, et seq.

Further provided is a pharmaceutical composition comprising a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in combination with a pharmaceutically acceptable diluent or carrier for use in treatment of a cell, tissue, or organ donor, e.g., for use in any of Methods A, A.1, et seq.

Phenylbenzamides or produgs thereof, e.g. of Formula I, Formula Ia, or 2-((3,5-bis(trifluoromethyl)phenyl)carbamoyl)-4-chlorophenyl dihydrogen phosphate, as hereinbefore described, may exist in free or salt form, e.g., as acid or base addition salts. In this specification, unless otherwise indicated, language such as “compound of Formula I” or “compound of Formula Ia” or “compound of Formula I or Formula Ia” or “compounds of Formula I or Formula Ia”, is to be understood as embracing the compounds in any form, for example free acid or base addition salt form. Pharmaceutically acceptable salts are known in the art and include salts which are physiologically acceptable at the dosage amount and form to be administered, for example tris(hydroxymethyl)aminomethane salts.

The term “prodrug” is a term of art which refers to a compound, which may be active or inactive itself as a pharmaceutical agent, but which under physiological conditions becomes converted to a desired active drug compound, e.g., a compound of Formula I. Typically, this conversion of a prodrug into its active drug involves the hydrolysis (chemically or enzymatically) of a chemical bond such that the prodrug disassociates into the active drug and a by-product. A pharmaceutically acceptable prodrug is one which undergoes this physiological hydrolysis at an acceptable rate in the desired biological tissue and which releases as a by-product a compound which is pharmaceutically acceptable, e.g., non-toxic at the expected dosage at which the prodrug is to be administered. Suitable prodrugs for the compound of Formula I include, but are not limited to, compounds of Formula I wherein the R₆ group is a physiologically hydrolysable and acceptable acyl or phosphono moiety. The benefits of using a prodrug rather than an active drug can be many. For example, the prodrug may be more metabolically stable, have improved pharmacokinetics (e.g., lower clearance, more desirable volume of distribution, more desirable membrane permeability), more desirable tissue localization, better patient tolerability (e.g., side effects), better shelf-life in a pharmaceutical composition, or improved ease of manufacture (e.g., cost, purity, or analysis). Where the physiologically hydrolysable prodrug moiety is itself susceptible to ionization and salt formation, the prodrug itself can form a salt, and this salt may be a pharmaceutically acceptable prodrug salt.

The term “patient” includes human or non-human (i.e., animal) patient. In a particular embodiment, the invention encompasses both human and nonhuman. In another embodiment, the invention encompasses nonhuman. In another embodiment, the term encompasses human.

The term “fairly rapid” with respect to onset of action means that the time it takes after a compound is administered for a response to be observed is 30 minutes or less, for example 20 minutes or less, for example or 15 minutes or less, for example 10 minutes or less, for example 5 minutes or less, for example 1 minute or less.

“Alkyl” is a saturated hydrocarbon moiety, preferably having one to six carbon atoms, preferably having one to four carbon atoms, which may be linear or branched. A “C₁₋₄-alkyl” is an alkyl having one to four carbon atoms.

“Alkylene” is a saturated hydrocarbon moiety, preferably having one to six carbon atoms, preferably having one to four carbon atoms, which may be linear or branched and which has two points of attachment. A C₁₋₄-alkylene is an alkylene having from one to four carbon atoms. For example, C₁-alkylene is methylene (—CH₂—).

“Carboxy” is —COOH.

“Hydroxy” is —OH.

Pharmaceutical compositions comprising 2-((3,5-bis(trifluoromethyl)phenyl)carbamoyl)-4-chlorophenyl dihydrogen phosphate (e.g., Compositions I and 1.1-1.124), salt solutions (e.g., Salt Solution I and 1.1-1.45), and methods of administration of 2-((3,5-bis(trifluoromethyl)phenyl)carbamoyl)-4-chlorophenyl dihydrogen phosphate which may be used in the methods described herein, e.g., Methods 1, 1.1, et seq, Methods 2, 2.1, et seq., Methods 3, 3.1, et seq., Methods 4, 4.1, et seq., Methods A, A.1, et seq., Methods 5, 5.1, et seq., Methods 6, 6.1, et seq. Methods 7, 7.1, et seq., Methods 8, 8.1, et seq., Methods 9, 9.1 et seq., Methods 10, 9.1 et seq., Methods 11, 9.1, et seq., Methods 12, 9.1 et seq., are described in International Application No. PCT/US14/64441, which is incorporated herein by reference in its entirety.

Methods of making and formulating compounds described herein are set forth in U.S. Patent Publication No. 2010/0274051 A1, U.S. Pat. Nos. 7,700,655 and 7,626,042, and International Application Nos. PCT/US14/64441 and PCT/US14/64447, each of which is incorporated herein by reference in its entirety.

Examples of acylated compounds and methods of making them are provided, e.g., in U.S. Patent Publication No. 2010/0274051 and U.S. Pat. Nos. 7,700,655 and 7,626,042, each of which is incorporated herein by reference in its entirety.

A dose or method of administration of the dose of the present invention is not particularly limited. Dosages employed in practicing the present invention will of course vary depending, e.g. on the particular disease or condition to be treated, the particular compound used, the mode of administration, and the therapy desired. The compounds may be administered by any suitable route, including orally, parenterally, transdermally, or by inhalation. In some cases, an IV infusion or IV bolus may be preferred. In general, satisfactory results, e.g. for the treatment of diseases as hereinbefore set forth are indicated to be obtained on oral administration at dosages of the order from about 0.01 to 15.0 mg/kg. In larger mammals, for example humans, an indicated daily dosage for oral administration will accordingly be in the range of from about 0.75 to 1000 mg per day, conveniently administered once, or in divided doses 2 to 3 times, daily or in sustained release form. Unit dosage forms for oral administration thus for example may comprise from about 0.2 to 75 or 150 mg, e.g. from about 0.2 or 2.0 to 50, 75, 100, 125, 150 or 200 mg of a phenylbenzamide or a pharmaceutically acceptable salt, prodrug, or pharmaceutically acceptable salt prodrug thereof, e.g., a compound of Formula I, Formula Ia, or 2-((3,5-bis(trifluoromethyl)phenyl)carbamoyl)-4-chlorophenyl dihydrogen phosphate, as hereinbefore described, together with a pharmaceutically acceptable diluent or carrier therefor. When the medicament is used via injection (subcutaneously, intramuscularly or intravenously) the dose may be 0.25 to 500 mg per day by bolus or if IV by bolus or infusion.

Further provided is a method (Method 5) of preservation of biological material, e.g., cell, tissue, or organ preservation, comprising contacting the biological material, e.g., the cell, tissue, or organ, e.g., the cell, e.g., the tissue, e.g., the organ, with a phenylbenzamide, e.g., a compound of Formula I:

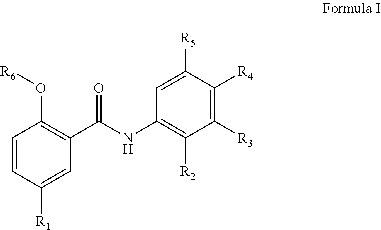

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
5.1. Method 5 wherein the phenylbenzamide is as described in U.S. Patent Publication No. 2010/0274051.
5.2. Method 5 wherein the phenylbenzamide is as described in U.S. Pat. Nos. 7,626,042 or 7,700,655.
5.3. Method 5 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
5.4. Method 5.3 wherein R₁ is selected from chloro and bromo; R₃ and R₅ are both trifluoromethyl; and R₂, R₄ and R₆ are all H, e.g., wherein the compound of Formula I is selected from:

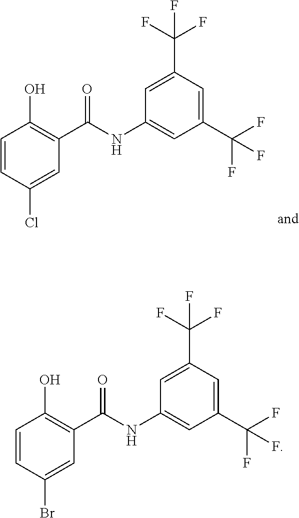 [SMILES: O=C(Nc1cc(C(F)(F)F)cc(C(F)(F)F)c1)c1cc(Cl)ccc1O ; O=C(Nc1cc(C(F)(F)F)cc(C(F)(F)F)c1)c1cc(Br)ccc1O]

5.5. Method 5 or 5.3 wherein R₆ is H or acetyl, e.g., H, e.g., acetyl.
5.6. Method 5 or 5.3 wherein R₁ is selected from chloro and bromo; R₃ and R₅ are both trifluoromethyl; and R₂ and R₄ are H and R₆ is acetyl, e.g., wherein the compound of Formula I is selected from:

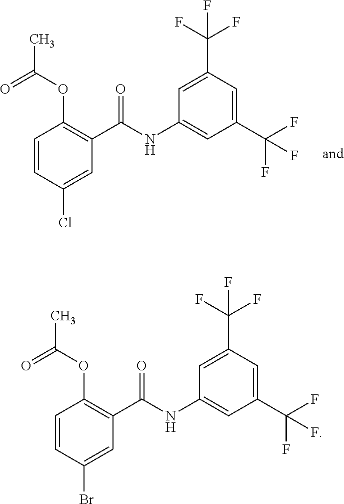 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

5.7. Method 5.4 wherein the compound of Formula I is:

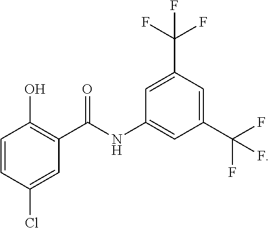 [SMILES: O=C(Nc1cc(C(F)(F)F)cc(C(F)(F)F)c1)c1cc(Cl)ccc1O]

5.8. Method 5.3 wherein R₁, R₃ and R₅ are each chloro, and R₂, R₄ and R₆ are each H.
5.9. Method 5.3 wherein R₁, R₃ and R₅ are each trifluoromethyl, and R₂, R₄ and R₆ are each H.
5.10. Method 5 or 5.3 wherein R₆ is C₁₋₄ acyl (e.g. acetyl).
5.11. Method 5 or 5.3 wherein R₆ is the residue of an amino acid.
5.12. Method 5 or 5.3 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
5.13. Method 5 or 5.3 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
5.14. Method 5 or 5.3 wherein R₆ is a (morpholin-4-yl)carbonyl group.
5.15. Method 5 or 5.3 wherein R₆ is a phosphono (—PO₃), which may be substituted, e.g. dibenzylphosphono (—P(═O)(OCH₂C₆H₅)₂), or unsubstituted (—P(═O)(OH)₂).
5.16. Method 5.15 wherein R₆ is ,,P(═O)(OH)₂.
5.17. Method 5.16 wherein the prodrug of Formula I is 2-((3,5-bis(trifluoromethyl)phenyl)carbamoyl)-4-chlorophenyl dihydrogen phosphate:

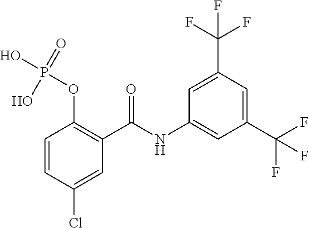 [SMILES: O=C(Nc1cc(C(F)(F)F)cc(C(F)(F)F)c1)c1cc(Cl)ccc1OP(=O)(O)O]

or a pharmaceutically acceptable salt thereof.
5.18. Method 5.15 wherein the prodrug of Formula I is:

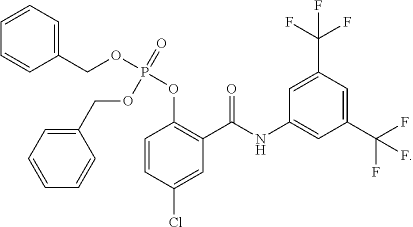 [SMILES: O=C(Nc1cc(C(F)(F)F)cc(C(F)(F)F)c1)c1cc(Cl)ccc1OP(=O)(OCc1ccccc1)OCc1ccccc1]

5.19. Method 5.15 wherein the pharmaceutically acceptable salt prodrug of Formula I is a compound of Formula Ia:

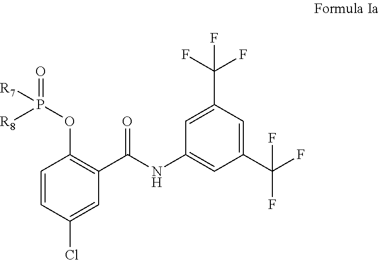

wherein one of R₇ and R₈ is OH and the other is O⁻Q⁺ or both R₇ and R₈ are O⁻Q⁺ wherein each Q⁺ is independently a pharmaceutically acceptable cation.
5.20. Method 5.19 wherein one of R₇ and R₈ is OH and the other is O⁻Q⁺.
5.21. Method 5.19 wherein both R₇ and R₈ are O⁻Q⁺.
5.22. Any of Method 5.19-5.21 wherein each Q⁺ is independently N⁺ or K⁺.
5.23. Method 5.22 wherein each Q⁺ is Na⁺.
5.24. Method 5.23 wherein the pharmaceutically acceptable salt prodrug of Formula Ia is:

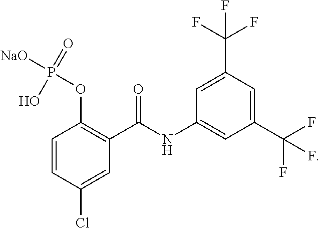

5.25. Method 5.23 wherein the pharmaceutically acceptable salt prodrug of Formula Ia is:

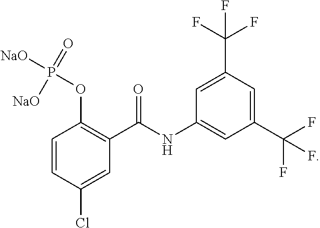

5.26. Any of Method 5.19-5.21 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂⁺, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
5.27. Method 5.26 wherein each Q⁺ is protonated tris(hydroxymethyl)aminomethane.
5.28. Any of Method 5.19-5.27 wherein Formula Ia is dissolved in a solution.
5.29. Method 5.28 wherein the concentration of

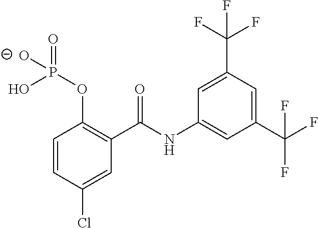 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
5.30. Method 5.28 wherein the concentration of

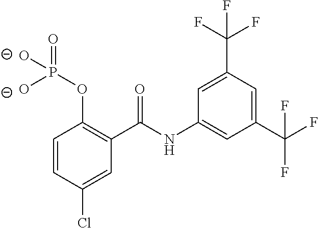 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
5.31. Method 5 wherein the compound of Formula I is:

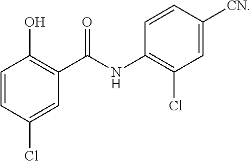 [SMILES: N#Cc1ccc(NC(=O)c2cc(Cl)ccc2O)c(Cl)c1]

5.32. Method 5 wherein the phenylbenzamide is:

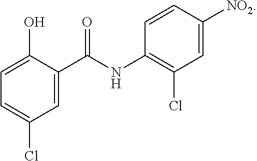 [SMILES: O=C(Nc1ccc([N+](=O)[O-])cc1Cl)c1cc(Cl)ccc1O]

5.33. Any of Method 5 et seq. wherein the compound of Formula I is

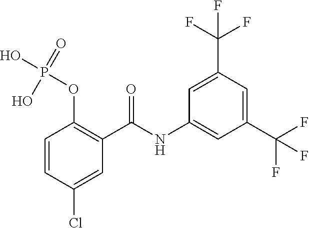 [SMILES: O=C(Nc1cc(C(F)(F)F)cc(C(F)(F)F)c1)c1cc(Cl)ccc1OP(=O)(O)O]

or a pharmaceutically acceptable salt thereof.
5.34. Any of Method 5 et seq. wherein the concentration of

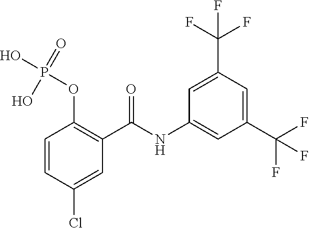 [SMILES: O=C(Nc1cc(C(F)(F)F)cc(C(F)(F)F)c1)c1cc(Cl)ccc1OP(=O)(O)O]

or a pharmaceutically acceptable salt thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
5.35. Any of Method 5 et seq. wherein the cell is a hematopoietic stem cell, lymphocyte, or pancreatic islet cell, e.g., hematopoietic stem cell, e.g., lymphocyte, e.g., pancreatic islet cell.
5.36. Any of Method 5 or 5.1-5.34 wherein the tissue is bone, tendon, cartilage, connective tissue, skin, cornea, sclera, heart valve, nerve, and vessel.
5.37. Any of Method 5 or 5.1-5.34 wherein the organ is a kidney.
5.38. Any of Method 5 or 5.1-5.34 wherein the organ is the liver.
5.39. Any of Method 5 or 5.1-5.34 wherein the organ is the pancreas.
5.40. Any of Method 5 or 5.1-5.34 wherein the organ is a lung.
5.41. Any of Method 5 or 5.1-5.34 wherein the organ is the heart.
5.42. Any of Method 5 or 5.1-5.34 wherein the organ is the thymus.
5.43. Any of Method 5 or 5.1-5.34 wherein the organ is the intestine.
5.44. Any of Method 5 or 5.1-5.34 wherein the organ is the uterus.
5.45. Any of Method 5 or 5.1-5.34 wherein the biological material is a face, limb (e.g., hand), eye, trachea, muscle, or esophagus.
5.46. Any of Method 5 et seq. wherein the aquaporin is AQP4.
5.47. Any of Method 5 et seq. wherein the aquaporin is AQP2.
5.48. Any of Method 5 et seq. wherein the biological material, e.g., cell, tissue, or organ, is perfused with the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof.
5.49. Any of Method 5 et seq. further comprising cooling, e.g., cold storage, e.g., cooling to 10° C. or less, e.g., 4° C. or less, e.g., 3° C. or less, e.g., 2, e.g., 1° C. or less, e.g., 0° C. or less, e.g., −6° C. or less, e.g., 0-10° C.
5.50. Any of Method 5 et seq. further comprising hypothermic perfusion.
5.51. Any of Method 5 et seq. comprising dissolving the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, in an aqueous solution.
5.52. Method 5.51 wherein the solution further comprises an osmotic active agent (e.g., lactogionate, raffinose, citrate, gluconate), an electrolyte (Na⁺, K⁺, Ca²⁺, Mg²⁺), an H⁺ ion buffer (phosphate, histidine, N-(2-hydroxyethyl)-piperazine-N′-2-ethanesulfonic acid (HEPES) buffer), a colloid (e.g., albumin, hydroxyethyl starch), a metabolic inhibitor (e.g., allopurinol, antiprotease, chlorpromazine), a metabolite (e.g., adenosine, glutathione), or an antioxidant (e.g., amino steroid, vitamin E, deferoxamine (Desferal), or a combination thereof.
5.53. Method 5.52 wherein the solution further comprises mannitol.
5.54. Method 5.52 or 5.53 wherein the solution is Collins solution, Euro-Collins solution, Ross-Marshall citrate solution, histidine tryptophan ketoglutarate solution, phosphate-buffered sucrose solution, University of Wisconsin solution, Celsior solution, Kyoto ET solution, or IGL-1 solution, e.g., University of Wisconsin solution.
5.55. Method 5.51 or 5.52 wherein the solution is Ringer's solution.
5.56. Any of Method 5 et seq. wherein the concentration of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
5.57. Any of Method 5 et seq. wherein the concentration of N-[3,5-bis(trifluoromethyl)phenyl]-5-chloro-2-hydroxybenzamide is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
5.58. Method 5 et seq. wherein the biological material, e.g., cell, tissue, or organ, remains viable for at least 72 hours, e.g., at least 48 hours, e.g., at least 36 hours, e.g., at least 30 hours, e.g., at least 24 hours, e.g., at least 18 hours, e.g., at least 16 hours, e.g., at least 12 hours, e.g., at least 8 hours, e.g., at least 6 hours.

Further provided is a method (Method 6) of preservation of biological material, e.g., cell, tissue, or organ preservation, comprising contacting the biological material, e.g., the cell, tissue, or organ, e.g., the cell, e.g., the tissue, e.g., the organ, with an effective amount of a an aquaporin inhibitor, e.g., a compound binding to an aquaporin, e.g., AQP4, e.g., an inhibitor of AQP2 or AQP4, e.g., AQP4, wherein the aquaporin inhibitor is a phenylbenzamide, e.g., a compound of Formula I:

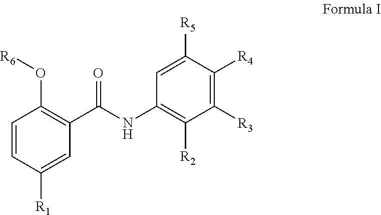

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
6.1. Method 6 wherein the phenylbenzamide is as described in U.S. Patent Publication No. 2010/0274051.
6.2. Method 6 wherein the phenylbenzamide is as described in U.S. Pat. Nos. 7,626,042 or 7,700,655.
6.3. Method 6 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
6.4. Method 6.3 wherein R₁ is selected from chloro and bromo; R₃ and R₅ are both trifluoromethyl; and R₂, R₄ and R₆ are all H, e.g., wherein the compound of Formula I is selected from:

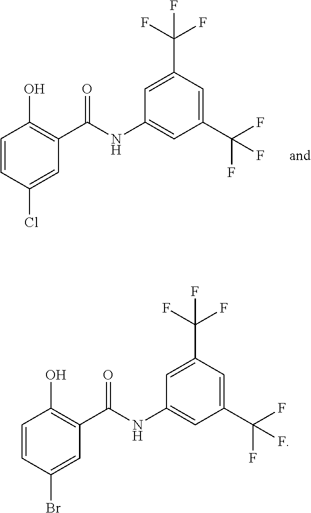 [SMILES: O=C(Nc1cc(C(F)(F)F)cc(C(F)(F)F)c1)c1cc(Cl)ccc1O ; O=C(Nc1cc(C(F)(F)F)cc(C(F)(F)F)c1)c1cc(Br)ccc1O]

6.5. Method 6 or 6.3 wherein R₆ is H or acetyl, e.g., H, e.g., acetyl.
6.6. Method 6 or 6.3 wherein R₁ is selected from chloro and bromo; R₃ and R₅ are both trifluoromethyl; and R₂ and R₄ are H and R₆ is acetyl, e.g., wherein the compound of Formula I is selected from:

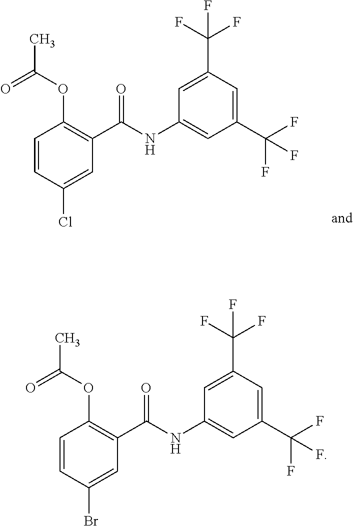 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

6.7. Method 6.4 wherein the compound of Formula I is:

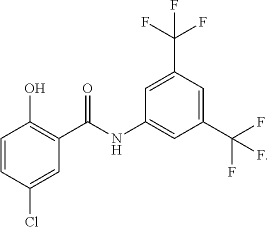 [SMILES: O=C(Nc1cc(C(F)(F)F)cc(C(F)(F)F)c1)c1cc(Cl)ccc1O]

6.8. Method 6.3 wherein R₁, R₃ and R₅ are each chloro, and R₂, R₄ and R₆ are each H.
6.9. Method 6.3 wherein R₁, R₃ and R₅ are each trifluoromethyl, and R₂, R₄ and R₆ are each H.
6.10. Method 6 or 6.3 wherein R₆ is C₁₋₄ acyl (e.g. acetyl).
6.11. Method 6 or 6.3 wherein R₆ is the residue of an amino acid.
6.12. Method 6 or 6.3 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
6.13. Method 6 or 6.3 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
6.14. Method 6 or 6.3 wherein R₆ is a (morpholin-4-yl)carbonyl group.
6.15. Method 6 or 6.3 wherein R₆ is a phosphono (—PO₃), which may be substituted, e.g. dibenzylphosphono (—P(═O)(OCH₂C₆H₅)₂), or unsubstituted (—P(═O)(OH)₂).
6.16. Method 6.15 wherein R₆ is ,,P(═O)(OH)₂.
6.17. Method 6.16 wherein the prodrug of Formula I is 2-((3,5-bis(trifluoromethyl)phenyl)carbamoyl)-4-chlorophenyl dihydrogen phosphate:

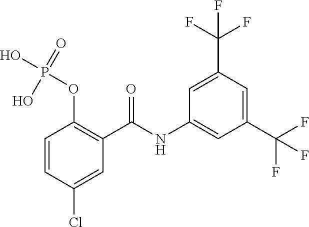 [SMILES: O=C(Nc1cc(C(F)(F)F)cc(C(F)(F)F)c1)c1cc(Cl)ccc1OP(=O)(O)O]

or a pharmaceutically acceptable salt thereof.
6.18. Method 6.15 wherein the prodrug of Formula I is:

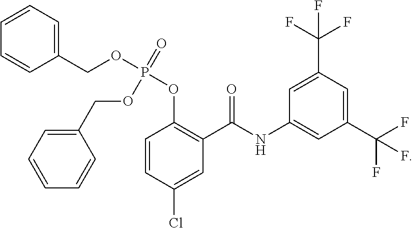 [SMILES: O=C(Nc1cc(C(F)(F)F)cc(C(F)(F)F)c1)c1cc(Cl)ccc1OP(=O)(OCc1ccccc1)OCc1ccccc1]

6.19. Method 6.15 wherein the pharmaceutically acceptable salt prodrug of Formula I is a compound of Formula Ia:

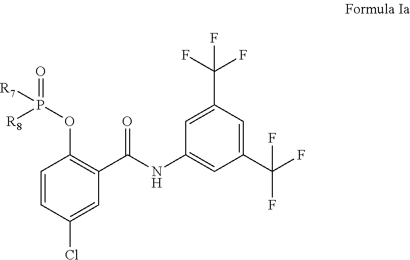

wherein one of R₇ and R₈ is OH and the other is O⁻Q⁺ or both R₇ and R₈ are O⁻Q⁺ wherein each Q⁺ is independently a pharmaceutically acceptable cation.
6.20. Method 6.19 wherein one of R₇ and R₈ is OH and the other is O⁻Q⁺.
6.21. Method 6.19 wherein both R₇ and R₈ are O⁻Q⁺.
6.22. Any of Method 6.19-6.21 wherein each Q⁺ is independently Na⁺ or K⁺.
6.23. Method 6.22 wherein each Q⁺ is Na⁺.
6.24. Method 6.23 wherein the pharmaceutically acceptable salt prodrug of Formula Ia is:

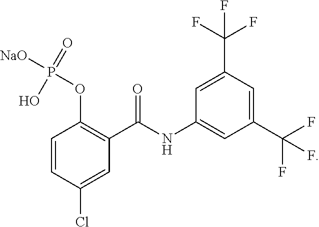

6.25. Method 6.23 wherein the pharmaceutically acceptable salt prodrug of Formula Ia is:

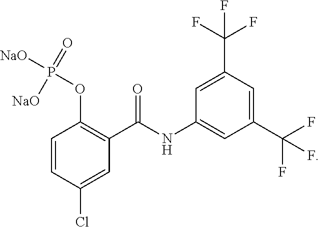

6.26. Any of Method 6.19-6.21 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
6.27. Method 6.26 wherein each Q⁺ is protonated tris(hydroxymethyl)aminomethane.
6.28. Any of Method 6.19-6.27 wherein Formula Ia is dissolved in a solution.
6.29. Method 6.28 wherein the concentration of

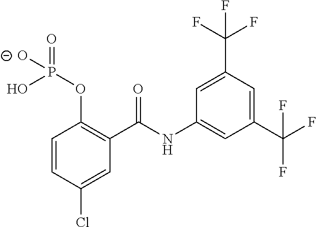 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
6.30. Method 6.28 wherein the concentration of

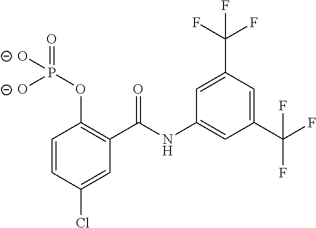 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
6.31. Method 6 wherein the compound of Formula I is:

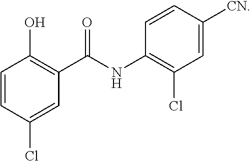 [SMILES: N#Cc1ccc(NC(=O)c2cc(Cl)ccc2O)c(Cl)c1]

6.32. Method 6 wherein the phenylbenzamide is:

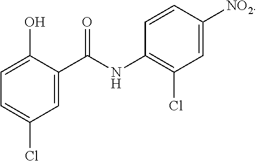 [SMILES: O=C(Nc1ccc([N+](=O)[O-])cc1Cl)c1cc(Cl)ccc1O]

6.33. Any of Method 6 et seq. wherein the compound of Formula I is

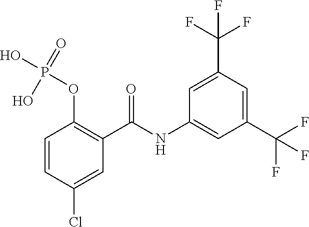 [SMILES: O=C(Nc1cc(C(F)(F)F)cc(C(F)(F)F)c1)c1cc(Cl)ccc1OP(=O)(O)O]

or a pharmaceutically acceptable salt thereof.
6.34. Any of Method 6 et seq. wherein the concentration of

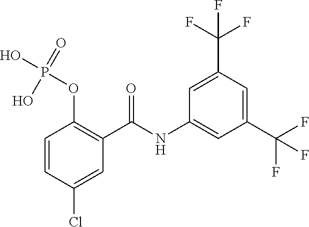 [SMILES: O=C(Nc1cc(C(F)(F)F)cc(C(F)(F)F)c1)c1cc(Cl)ccc1OP(=O)(O)O]

or a pharmaceutically acceptable salt thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
6.35. Any of Method 6 et seq. wherein the cell is a hematopoietic stem cell, lymphocyte, or pancreatic islet cell, e.g., hematopoietic stem cell, e.g., lymphocyte, e.g., pancreatic islet cell.
6.36. Any of Method 6 or 6.1-6.34 wherein the tissue is bone, tendon, cartilage, connective tissue, skin, cornea, sclera, heart valve, nerve, and vessel.
6.37. Any of Method 6 or 6.1-6.34 wherein the organ is a kidney.
6.38. Any of Method 6 or 6.1-6.34 wherein the organ is the liver.
6.39. Any of Method 6 or 6.1-6.34 wherein the organ is the pancreas.
6.40. Any of Method 6 or 6.1-6.34 wherein the organ is a lung.
6.41. Any of Method 6 or 6.1-6.34 wherein the organ is the heart.
6.42. Any of Method 6 or 6.1-6.34 wherein the organ is the thymus.
6.43. Any of Method 6 or 6.1-6.34 wherein the organ is the intestine.
6.44. Any of Method 6 or 6.1-6.34 wherein the organ is the uterus.
6.45. Any of Method 6 or 6.1-6.34 wherein the biological material is a face, limb (e.g., hand), eye, trachea, muscle, or esophagus.
6.46. Any of Method 6 et seq. wherein the aquaporin is AQP4.
6.47. Any of Method 6 et seq. wherein the aquaporin is AQP2.
6.48. Any of Method 6 et seq. wherein the biological material, e.g., cell, tissue, or organ, is perfused with the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof.
6.49. Any of Method 6 et seq. further comprising cooling, e.g., cold storage, e.g., cooling to 10° C. or less, e.g., 4° C. or less, e.g., 3° C. or less, e.g., 2, e.g., 1° C. or less, e.g., 0° C. or less, e.g., −6° C. or less, e.g., 0-10° C.
6.50. Any of Method 6 et seq. further comprising hypothermic perfusion.
6.51. Any of Method 6 et seq. comprising dissolving the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, in an aqueous solution.
6.52. Method 6.51 wherein the solution further comprises an osmotic active agent (e.g., lactogionate, raffinose, citrate, gluconate), an electrolyte (Na⁺, K⁺, Ca²⁺, Mg²⁺), an H⁺ ion buffer (phosphate, histidine, N-(2-hydroxyethyl)-piperazine-N′-2-ethanesulfonic acid (HEPES) buffer), a colloid (e.g., albumin, hydroxyethyl starch), a metabolic inhibitor (e.g., allopurinol, antiprotease, chlorpromazine), a metabolite (e.g., adenosine, glutathione), or an antioxidant (e.g., amino steroid, vitamin E, deferoxamine (Desferal), or a combination thereof.
6.53. Method 6.52 wherein the solution further comprises mannitol.
6.54. Method 6.52 wherein the solution is Collins solution, Euro-Collins solution, Ross-Marshall citrate solution, histidine tryptophan ketoglutarate solution, phosphate-buffered sucrose solution, University of Wisconsin solution, Celsior solution, Kyoto ET solution, or IGL-1 solution, e.g., University of Wisconsin solution.
6.55. Method 6.51 or 6.52 wherein the solution is Ringer's solution.
6.56. Any of Method 6 et seq. wherein the concentration of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
6.57. Any of Method 6 et seq. wherein the concentration of N-[3,5-bis(trifluoromethyl)phenyl]-5-chloro-2-hydroxybenzamide is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
6.58. Method 6 et seq. wherein the biological material, e.g., cell, tissue, or organ, remains viable for at least 72 hours, e.g., at least 48 hours, e.g., at least 36 hours, e.g., at least 30 hours, e.g., at least 24 hours, e.g., at least 18 hours, e.g., at least 16 hours, e.g., at least 12 hours, e.g., at least 8 hours, e.g., at least 6 hours.

Further provided is a method (Method 7) of preservation of biological material, e.g., cell, tissue, or organ preservation, comprising contacting the biological material, e.g., the cell, tissue, or organ, e.g., the cell, e.g., the tissue, e.g., the organ, with an aquaporin inhibitor, e.g., a compound binding to an aquaporin, e.g., AQP4, e.g., an inhibitor of AQP2 or AQP4, e.g., AQP4, in an amount effective to inhibit the aquaporin, wherein the aquaporin inhibitor is a phenylbenzamide, e.g., a compound of Formula I:

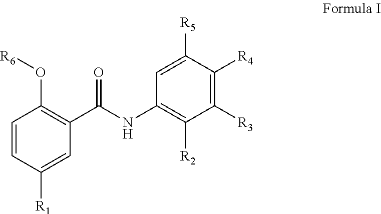

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
7.1. Method 7 wherein the phenylbenzamide is as described in U.S. Patent Publication No. 2010/0274051.
7.2. Method 7 wherein the phenylbenzamide is as described in U.S. Pat. Nos. 7,626,042 or 7,700,655.
7.3. Method 7 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
7.4. Method 7.3 wherein R₁ is selected from chloro and bromo; R₃ and R₅ are both trifluoromethyl; and R₂, R₄ and R₆ are all H, e.g., wherein the compound of Formula I is selected from:

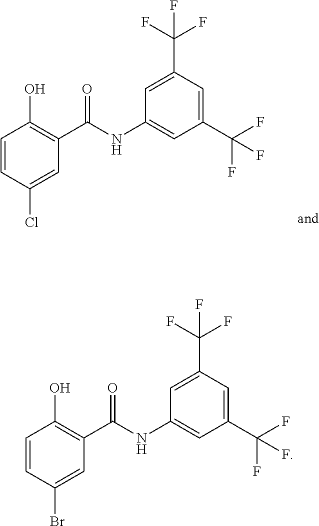 [SMILES: O=C(Nc1cc(C(F)(F)F)cc(C(F)(F)F)c1)c1cc(Cl)ccc1O ; O=C(Nc1cc(C(F)(F)F)cc(C(F)(F)F)c1)c1cc(Br)ccc1O]

7.5. Method 7 or 7.3 wherein R₆ is H or acetyl, e.g., H, e.g., acetyl.
7.6. Method 7 or 7.3 wherein R₁ is selected from chloro and bromo; R₃ and R₅ are both trifluoromethyl; and R₂ and R₄ are H and R₆ is acetyl, e.g., wherein the compound of Formula I is selected from:

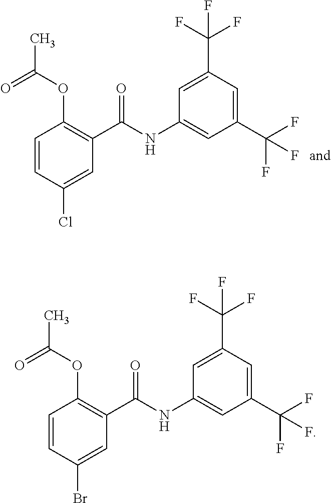 [SMILES: CC(=O)Oc1ccc(Cl)cc1C(=O)Nc1cc(C(F)(F)F)cc(C(F)(F)F)c1 ; "and" ; CC(=O)Oc1ccc(Br)cc1C(=O)Nc1cc(C(F)(F)F)cc(C(F)(F)F)c1]

7.7. Method 7.4 wherein the compound of Formula I is:

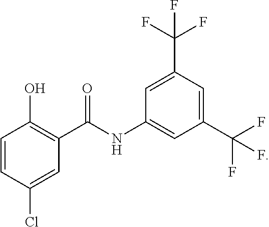 [SMILES: O=C(Nc1cc(C(F)(F)F)cc(C(F)(F)F)c1)c1cc(Cl)ccc1O]

7.8. Method 7.3 wherein R₁, R₃ and R₅ are each chloro, and R₂, R₄ and R₆ are each H.
7.9. Method 7.3 wherein R₁, R₃ and R₅ are each trifluoromethyl, and R₂, R₄ and R₆ are H.
7.10. Method 7 or 7.3 wherein R₆ is C₁₋₄ acyl (e.g. acetyl).
7.11. Method 7 or 7.3 wherein R₆ is the residue of an amino acid.
7.12. Method 7 or 7.3 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
7.13. Method 7 or 7.3 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
7.14. Method 7 or 7.3 wherein R₆ is a (morpholin-4-yl)carbonyl group.
7.15. Method 7 or 7.3 wherein R₆ is a phosphono (—PO₃), which may be substituted, e.g. dibenzylphosphono (—P(═O)(OCH₂C₆H₅)₂), or unsubstituted (—P(═O)(OH)₂).
7.16. Method 7.15 wherein R₆ is ,,P(═O)(OH)₂.
7.17. Method 7.16 wherein the prodrug of Formula I is 2-((3,5-bis(trifluoromethyl)phenyl)carbamoyl)-4-chlorophenyl dihydrogen phosphate:

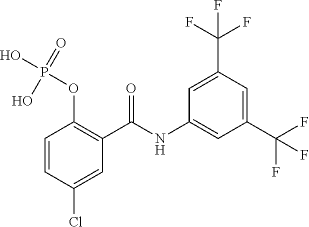 [SMILES: O=C(Nc1cc(C(F)(F)F)cc(C(F)(F)F)c1)c1cc(Cl)ccc1OP(=O)(O)O]

or a pharmaceutically acceptable salt thereof.
7.18. Method 7.15 wherein the prodrug of Formula I is:

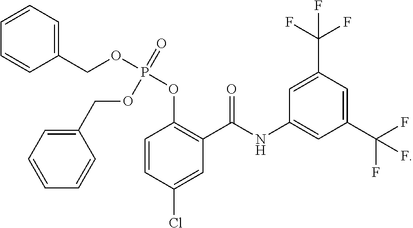 [SMILES: O=C(Nc1cc(C(F)(F)F)cc(C(F)(F)F)c1)c1cc(Cl)ccc1OP(=O)(OCc1ccccc1)OCc1ccccc1]

7.19. Method 7.15 wherein the pharmaceutically acceptable salt prodrug of Formula I is a compound of Formula Ia:

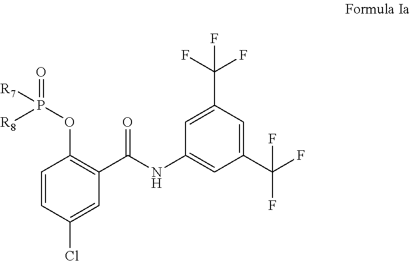

wherein one of R₇ and R₈ is OH and the other is O⁻Q⁺ or both R₇ and R₈ are O⁻Q⁺ wherein each Q⁺ is independently a pharmaceutically acceptable cation.
7.20. Method 7.19 wherein one of R₇ and R₈ is OH and the other is O⁻Q⁺.
7.21. Method 7.19 wherein both R₇ and R₈ are O⁻Q⁺.
7.22. Any of Method 7.19-7.21 wherein each Q⁺ is independently N⁺ or K⁺.
7.23. Method 7.22 wherein each Q⁺ is Na⁺.
7.24. Method 7.23 wherein the pharmaceutically acceptable salt prodrug of Formula Ia is:

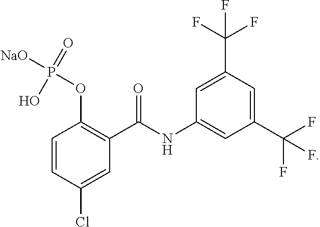

7.25. Method 7.23 wherein the pharmaceutically acceptable salt prodrug of Formula Ia is:

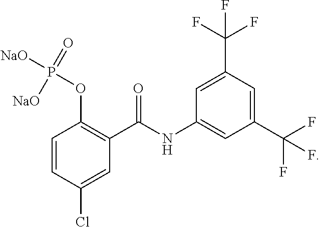

7.26. Any of Method 7.19-7.21 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂⁺, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
7.27. Method 7.26 wherein each Q⁺ is protonated tris(hydroxymethyl)aminomethane.
7.28. Any of Method 7.19-7.27 wherein Formula Ia is dissolved in a solution.
7.29. Method 7.28 wherein the concentration of

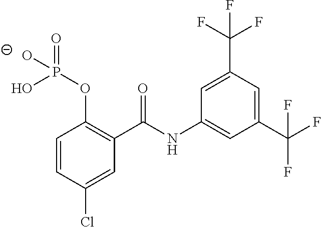 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
7.30. Method 7.28 wherein the concentration of

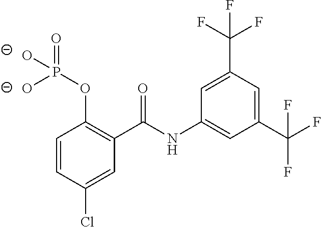 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
7.31. Method 7 wherein the compound of Formula I is:

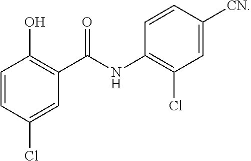 [SMILES: N#Cc1ccc(NC(=O)c2cc(Cl)ccc2O)c(Cl)c1]

7.32. Method 7 wherein the phenylbenzamide is:

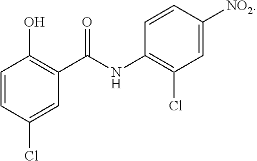 [SMILES: O=C(Nc1ccc([N+](=O)[O-])cc1Cl)c1cc(Cl)ccc1O]

7.33. Any of Method 7 et seq. wherein the compound of Formula I is

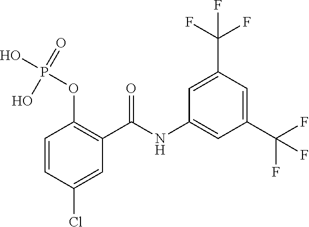 [SMILES: O=C(Nc1cc(C(F)(F)F)cc(C(F)(F)F)c1)c1cc(Cl)ccc1OP(=O)(O)O]

or a pharmaceutically acceptable salt thereof.
7.34. Any of Method 7 et seq. wherein the concentration of

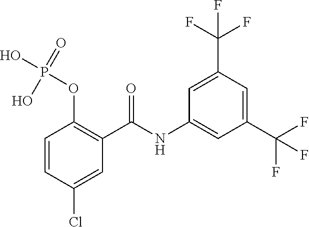 [SMILES: O=C(Nc1cc(C(F)(F)F)cc(C(F)(F)F)c1)c1cc(Cl)ccc1OP(=O)(O)O]

or a pharmaceutically acceptable salt thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
7.35. Any of Method 7 et seq. wherein the cell is a hematopoietic stem cell, lymphocyte, or pancreatic islet cell, e.g., hematopoietic stem cell, e.g., lymphocyte, e.g., pancreatic islet cell.
7.36. Any of Method 7 or 7.1-7.34 wherein the tissue is bone, tendon, cartilage, connective tissue, skin, cornea, sclera, heart valve, nerve, and vessel.
7.37. Any of Method 7 or 7.1-7.34 wherein the organ is a kidney.
7.38. Any of Method 7 or 7.1-7.34 wherein the organ is the liver.
7.39. Any of Method 7 or 7.1-7.34 wherein the organ is the pancreas.
7.40. Any of Method 7 or 7.1-7.34 wherein the organ is a lung.
7.41. Any of Method 7 or 7.1-7.34 wherein the organ is the heart.
7.42. Any of Method 7 or 7.1-7.34 wherein the organ is the thymus.
7.43. Any of Method 7 or 7.1-7.34 wherein the organ is the intestine.
7.44. Any of Method 7 or 7.1-7.34 wherein the organ is the uterus.
7.45. Any of Method 7 or 7.1-7.34 wherein the biological material is a face, limb (e.g., hand), eye, trachea, muscle, or esophagus.
7.46. Any of Method 7 et seq. wherein the aquaporin is AQP4.
7.47. Any of Method 7 et seq. wherein the aquaporin is AQP2.
7.48. Any of Method 7 et seq. wherein the biological material, e.g., cell, tissue, or organ, is perfused with the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof.
7.49. Any of Method 7 et seq. further comprising cooling, e.g., cold storage, e.g., cooling to 10° C. or less, e.g., 4° C. or less, e.g., 3° C. or less, e.g., 2, e.g., 1° C. or less, e.g., 0° C. or less, e.g., −6° C. or less, e.g., 0-10° C.
7.50. Any of Method 7 et seq. further comprising hypothermic perfusion.
7.51. Any of Method 7 et seq. comprising dissolving the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, in an aqueous solution.
7.52. Method 7.51 wherein the solution further comprises an osmotic active agent (e.g., lactogionate, raffinose, citrate, gluconate), an electrolyte (N^{a}, K+, Ca²⁺, Mg²⁺), an H⁺ ion buffer (phosphate, histidine, N-(2-hydroxyethyl)-piperazine-N′-2-ethanesulfonic acid (HEPES) buffer), a colloid (e.g., albumin, hydroxyethyl starch), a metabolic inhibitor (e.g., allopurinol, antiprotease, chlorpromazine), a metabolite (e.g., adenosine, glutathione), or an antioxidant (e.g., amino steroid, vitamin E, deferoxamine (Desferal), or a combination thereof.
7.53. Method 7.52 wherein the solution further comprises mannitol.
7.54. Method 7.52 wherein the solution is Collins solution, Euro-Collins solution, Ross-Marshall citrate solution, histidine tryptophan ketoglutarate solution, phosphate-buffered sucrose solution, University of Wisconsin solution, Celsior solution, Kyoto ET solution, or IGL-1 solution, e.g., University of Wisconsin solution.
7.55. Method 7.51 or 7.52 wherein the solution is Ringer's solution.
7.56. Any of Method 7 et seq. wherein the concentration of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
7.57. Any of Method 7 et seq. wherein the concentration of N-[3,5-bis(trifluoromethyl)phenyl]-5-chloro-2-hydroxybenzamide is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
7.58. Method 7 et seq. wherein the biological material, e.g., cell, tissue, or organ, remains viable for at least 72 hours, e.g., at least 48 hours, e.g., at least 36 hours, e.g., at least 30 hours, e.g., at least 24 hours, e.g., at least 18 hours, e.g., at least 16 hours, e.g., at least 12 hours, e.g., at least 8 hours, e.g., at least 6 hours.

Further provided is a method (Method 8) to inhibit an aquaporin to preserve biological material, e.g., cell, tissue, or organ preservation, comprising contacting the biological material, e.g., the cell, tissue, or organ, e.g., the cell, e.g., the tissue, e.g., the organ, with an effective amount of an aquaporin inhibitor, e.g., a compound binding to an aquaporin, e.g., AQP4, e.g., an inhibitor of AQP2 or AQP4, e.g., AQP4, in an amount effective to inhibit the aquaporin, wherein the aquaporin inhibitor is a phenylbenzamide, e.g., a compound of Formula I:

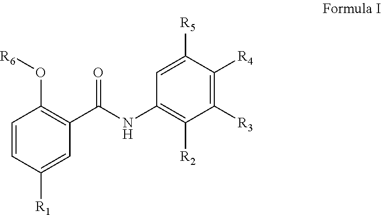

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof, for example,
8.1. Method 8 wherein the phenylbenzamide is as described in U.S. Patent Publication No. 2010/0274051.
8.2. Method 8 wherein the phenylbenzamide is as described in U.S. Pat. Nos. 7,626,042 or 7,700,655.
8.3. Method 8 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
8.4. Method 8.3 wherein R₁ is selected from chloro and bromo; R₃ and R₅ are both trifluoromethyl; and R₂, R₄ and R₆ are all H, e.g., wherein the compound of Formula I is selected from:

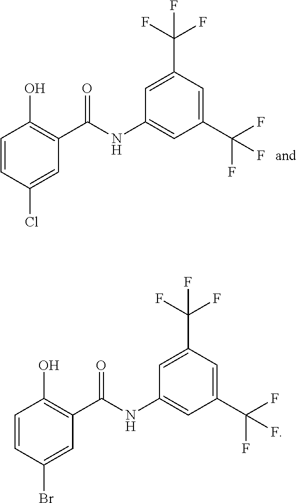 [SMILES: O=C(Nc1cc(C(F)(F)F)cc(C(F)(F)F)c1)c1cc(Cl)ccc1O ; "and" ; O=C(Nc1cc(C(F)(F)F)cc(C(F)(F)F)c1)c1cc(Br)ccc1O]

8.5. Method 8 or 8.3 wherein R₆ is H or acetyl, e.g., H, e.g., acetyl.
8.6. Method 8 or 8.3 wherein R₁ is selected from chloro and bromo; R₃ and R₅ are both trifluoromethyl; and R₂ and R₄ are H and R₆ is acetyl, e.g., wherein the compound of Formula I is selected from:

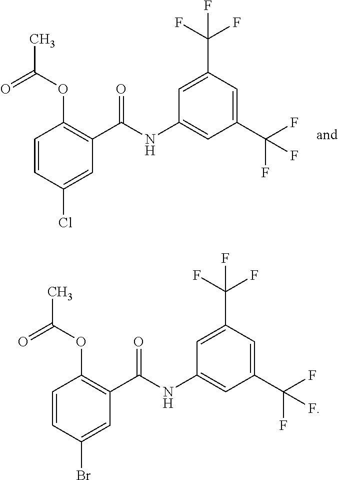 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

8.7. Method 8.4 wherein the compound of Formula I is:

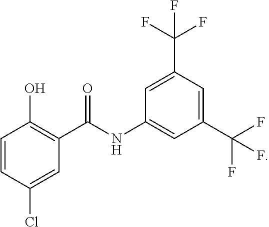 [SMILES: O=C(Nc1cc(C(F)(F)F)cc(C(F)(F)F)c1)c1cc(Cl)ccc1O]

8.8. Method 8.3 wherein R₁, R₃ and R₅ are each chloro, and R₂, R₄ and R₆ are each H.
8.9. Method 8.3 wherein R₁, R₃ and R₅ are each trifluoromethyl, and R₂, R₄ and R₆ are each H.
8.10. Method 8 or 8.3 wherein R₆ is C₁₋₄ acyl (e.g. acetyl).
8.11. Method 8 or 8.3 wherein R₆ is the residue of an amino acid.
8.12. Method 8 or 8.3 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
8.13. Method 8 or 8.3 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
8.14. Method 8 or 8.3 wherein R₆ is a (morpholin-4-yl)carbonyl group.
8.15. Method 8 or 8.3 wherein R₆ is a phosphono (—PO₃), which may be substituted, e.g. dibenzylphosphono (—P(═O)(OCH₂C₆H₅)₂), or unsubstituted (—P(═O)(OH)₂).
8.16. Method 8.15 wherein R₆ is ,,P(═O)(OH)₂.
8.17. Method 8.16 wherein the prodrug of Formula I is 2-((3,5-bis(trifluoromethyl)phenyl)carbamoyl)-4-chlorophenyl dihydrogen phosphate:

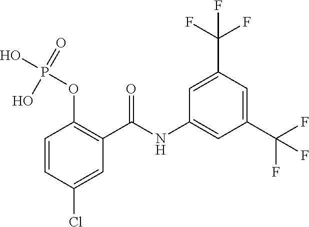 [SMILES: O=C(Nc1cc(C(F)(F)F)cc(C(F)(F)F)c1)c1cc(Cl)ccc1OP(=O)(O)O]

or a pharmaceutically acceptable salt thereof.
8.18. Method 8.15 wherein the prodrug of Formula I is:

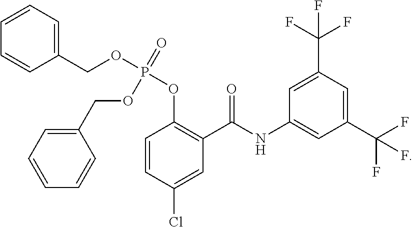 [SMILES: O=C(Nc1cc(C(F)(F)F)cc(C(F)(F)F)c1)c1cc(Cl)ccc1OP(=O)(OCc1ccccc1)OCc1ccccc1]

8.19. Method 8.15 wherein the pharmaceutically acceptable salt prodrug of Formula I is a compound of Formula Ia:

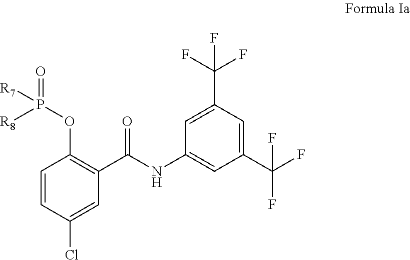

wherein one of R₇ and R₈ is OH and the other is O⁻Q⁺ or both R₇ and R₈ are O⁻Q⁺ wherein each Q⁺ is independently a pharmaceutically acceptable cation.
8.20. Method 8.19 wherein one of R₇ and R₈ is OH and the other is O⁻Q⁺.
8.21. Method 8.19 wherein both R₇ and R₈ are O⁻Q⁺.
8.22. Any of Method 8.19-8.21 wherein each Q⁺ is independently N⁺ or K⁺.
8.23. Method 8.22 wherein each Q⁺ is Na⁺.
8.24. Method 8.23 wherein the pharmaceutically acceptable salt prodrug of Formula Ia is:

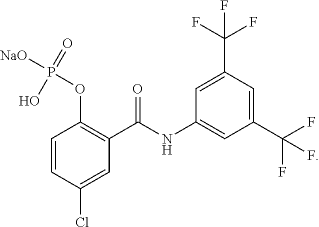

8.25. Method 8.23 wherein the pharmaceutically acceptable salt prodrug of Formula Ia is:

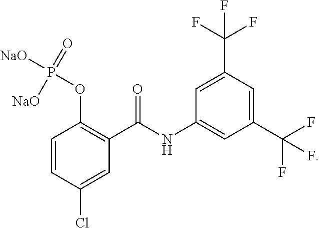

8.26. Any of Method 8.19-8.21 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
8.27. Method 8.26 wherein each Q⁺ is protonated tris(hydroxymethyl)aminomethane.
8.28. Any of Method 8.19-8.27 wherein Formula Ia is dissolved in a solution.
8.29. Method 8.28 wherein the concentration of

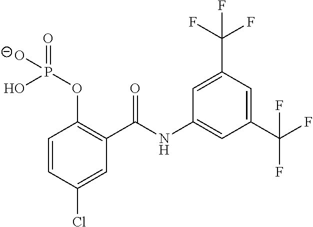 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
8.30. Method 8.28 wherein the concentration of

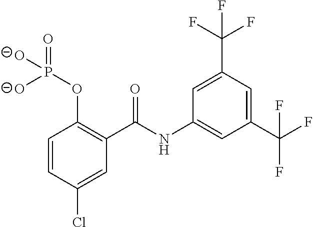 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
8.31. Method 8 wherein the compound of Formula I is:

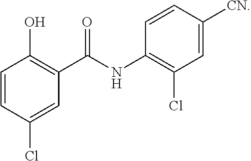 [SMILES: N#Cc1ccc(NC(=O)c2cc(Cl)ccc2O)c(Cl)c1]

8.32. Method 8 wherein the phenylbenzamide is:

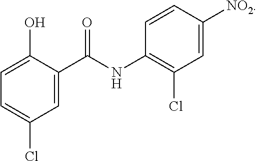 [SMILES: O=C(Nc1ccc([N+](=O)[O-])cc1Cl)c1cc(Cl)ccc1O]

8.33. Any of Method 8 et seq. wherein the compound of Formula I is

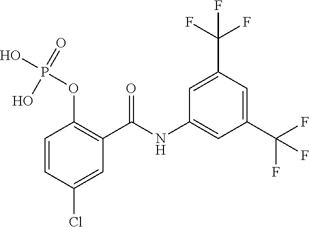 [SMILES: O=C(Nc1cc(C(F)(F)F)cc(C(F)(F)F)c1)c1cc(Cl)ccc1OP(=O)(O)O]

or a pharmaceutically acceptable salt thereof.
8.34. Any of Method 8 et seq. wherein the concentration of

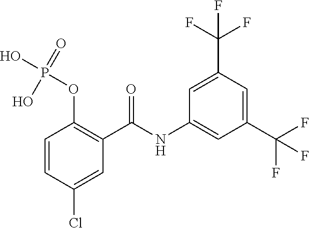 [SMILES: O=C(Nc1cc(C(F)(F)F)cc(C(F)(F)F)c1)c1cc(Cl)ccc1OP(=O)(O)O]

or a pharmaceutically acceptable salt thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
8.35. Any of Method 8 et seq. wherein the cell is a hematopoietic stem cell, lymphocyte, or pancreatic islet cell, e.g., hematopoietic stem cell, e.g., lymphocyte, e.g., pancreatic islet cell.
8.36. Any of Method 8 or 8.1-8.34 wherein the tissue is bone, tendon, cartilage, connective tissue, skin, cornea, sclera, heart valve, nerve, and vessel.
8.37. Any of Method 8 or 8.1-8.34 wherein the organ is a kidney.
8.38. Any of Method 8 or 8.1-8.34 wherein the organ is the liver.
8.39. Any of Method 8 or 8.1-8.34 wherein the organ is the pancreas.
8.40. Any of Method 8 or 8.1-8.34 wherein the organ is a lung.
8.41. Any of Method 8 or 8.1-8.34 wherein the organ is the heart.
8.42. Any of Method 8 or 8.1-8.34 wherein the organ is the thymus.
8.43. Any of Method 8 or 8.1-8.34 wherein the organ is the intestine.
8.44. Any of Method 8 or 8.1-8.34 wherein the organ is the uterus.
8.45. Any of Method 8 or 8.1-8.34 wherein the biological material is a face, limb (e.g., hand), eye, trachea, muscle, or esophagus.
8.46. Any of Method 8 et seq. wherein the aquaporin is AQP4.
8.47. Any of Method 8 et seq. wherein the aquaporin is AQP2.
8.48. Any of Method 8 et seq. wherein the biological material, e.g., cell, tissue, or organ, is perfused with the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof.
8.49. Any of Method 8 et seq. further comprising cooling, e.g., cold storage, e.g., cooling to 10° C. or less, e.g., 4° C. or less, e.g., 3° C. or less, e.g., 2, e.g., 1° C. or less, e.g., 0° C. or less, e.g., −6° C. or less, e.g., 0-10° C.
8.50. Any of Method 8 et seq. further comprising hypothermic perfusion.
8.51. Any of Method 8 et seq. comprising dissolving the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, in an aqueous solution.
8.52. Method 8.51 wherein the solution further comprises an osmotic active agent (e.g., lactogionate, raffinose, citrate, gluconate), an electrolyte (Na⁺, K⁺, Ca²⁺, Mg²⁺), an H⁺ ion buffer (phosphate, histidine, N-(2-hydroxyethyl)-piperazine-N′-2-ethanesulfonic acid (HEPES) buffer), a colloid (e.g., albumin, hydroxyethyl starch), a metabolic inhibitor (e.g., allopurinol, antiprotease, chlorpromazine), a metabolite (e.g., adenosine, glutathione), or an antioxidant (e.g., amino steroid, vitamin E, deferoxamine (Desferal), or a combination thereof.
8.53. Method 8.52 wherein the solution further comprises mannitol.
8.54. Method 8.52 wherein the solution is Collins solution, Euro-Collins solution, Ross-Marshall citrate solution, histidine tryptophan ketoglutarate solution, phosphate-buffered sucrose solution, University of Wisconsin solution, Celsior solution, Kyoto ET solution, or IGL-1 solution, e.g., University of Wisconsin solution.
8.55. Method 8.51 or 8.52 wherein the solution is Ringer's solution.
8.56. Any of Method 8 et seq. wherein the concentration of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
8.57. Any of Method 8 et seq. wherein the concentration of N-[3,5-bis(trifluoromethyl)phenyl]-5-chloro-2-hydroxybenzamide is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
8.58. Method 8 et seq. wherein the biological material, e.g., cell, tissue, or organ, remains viable for at least 72 hours, e.g., at least 48 hours, e.g., at least 36 hours, e.g., at least 30 hours, e.g., at least 24 hours, e.g., at least 18 hours, e.g., at least 16 hours, e.g., at least 12 hours, e.g., at least 8 hours, e.g., at least 6 hours.

In a further embodiment, the invention provides a method for treatment or prophylaxis of transplant rejection, inhibiting rejection of transplanted biological material, or prophylaxis, treatment, or control of edema consequent to a transplant (e.g a method according to any of Methods 1, et seq., 2, et seq., 3, et seq. or 4, et seq.) wherein prior to transplantation, the biological material to be transplanted is treated in accordance with any of Methods 5, et seq., 6, et seq., 7, et seq., or 8, et seq.

Further provided is a method (Method 9) of protecting a heart during heart surgery, e.g., open heart surgery, comprising contacting the heart of a patient in need thereof with a phenylbenzamide, e.g., a compound of Formula I (as described below), before, during, and/or after the surgery. As used herein, “protecting” refers to any action taken to prevent or ameliorate damage occurring to the heart tissue during heart surgery. This includes, but is not limited to, preventing or ameliorating cellular edema, hypoxia, apoptosis, necrosis or dysfunction, e.g., dysfunction of electrical conduction, contraction, or metabolism.

Further provided is a method (Method 10) of protecting a heart during heart surgery, e.g., open heart surgery, comprising contacting the heart of a patient in need thereof with an effective amount of an aquaporin inhibitor, e.g., a compound binding to an aquaporin, e.g., AQP4, e.g., an inhibitor of AQP2 or AQP4, e.g., AQP4, wherein the aquaporin inhibitor is a phenylbenzamide, e.g., a compound of Formula I (as described below), before, during, and/or after the surgery.

Further provided is a method (Method 11) of protecting the heart during heart surgery, e.g., open heart surgery, with an aquaporin inhibitor, e.g., a compound binding to an aquaporin, e.g., AQP4, e.g., an inhibitor of AQP2 or AQP4, e.g., AQP4, comprising contacting the heart of a patient in need thereof with the aquaporin inhibitor in an amount effective to inhibit the aquaporin before, during, and/or after surgery, wherein the aquaporin inhibitor is a phenylbenzamide, e.g., a compound of Formula I (as described below).

Further provided is a method (Method 12) to inhibit an aquaporin to protect a heart during heart surgery, e.g., open heart surgery, comprising administering to a patient in need thereof before, during, and/or after surgery an aquaporin inhibitor, e.g., a compound binding to an aquaporin, e.g. AQP4, e.g., an inhibitor of AQP2 or AQP4, e.g., AQP4, in an amount effective to inhibit the aquaporin, e.g., AQP4, wherein the aquaporin inhibitor is a phenylbenzamide, e.g., a compound of Formula I (as described below).

A compound of Formula I for use in any of Methods 9, 10, 11, or 12 is as follows:

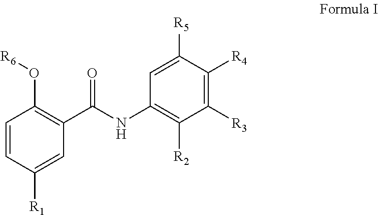

wherein R₁, R₂, R₃, R₄, and R₅ are selected from H, halogen, halogenated C₁₋₄ alkyl (e.g., trifluoromethyl), and cyano; and

### R₆ is H;

or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof.

In another embodiment, further provided are Methods 9, 10, 11, and 12 as follows:
9.1. Any of Method 9, 10, 11 or 12 wherein the phenylbenzamide is as described in U.S. Patent Publication No. 2010/0274051.
9.2. Any of Method 9, 10, 11, or 12 wherein the phenylbenzamide is as described in U.S. Pat. Nos. 7,626,042 or 7,700,655.
9.3. Any of Method 9, 10, 11, or 12 wherein R₁ is selected from trifluoromethyl, chloro, fluoro, and bromo; R₃ and R₅ are the same or different and selected from trifluoromethyl, chloro, fluoro, and bromo; and R₂ and R₄ are both H.
9.4. Method 9.3 wherein R₁ is selected from chloro and bromo; R₃ and R₅ are both trifluoromethyl; and R₂, R₄ and R₆ are all H, e.g., wherein the compound of Formula I is selected from:

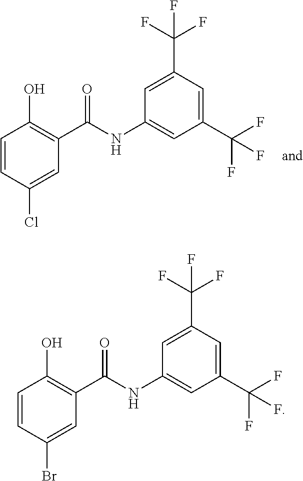 [SMILES: O=C(Nc1cc(C(F)(F)F)cc(C(F)(F)F)c1)c1cc(Cl)ccc1O ; "and" ; O=C(Nc1cc(C(F)(F)F)cc(C(F)(F)F)c1)c1cc(Br)ccc1O]

9.5. Any of Method 9, 10, 11, or 12 or 9.3 wherein R₆ is H or acetyl, e.g., H, e.g., acetyl.
9.6. Any of Method 9, 10, 11, or 12 or 9.3 wherein R₁ is selected from chloro and bromo; R₃ and R₅ are both trifluoromethyl; and R₂ and R₄ are H and R₆ is acetyl, e.g., wherein the compound of Formula I is selected from:

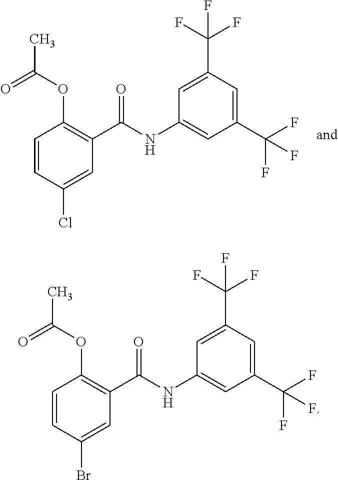 [SMILES: CC(=O)Oc1ccc(Cl)cc1C(=O)Nc1cc(C(F)(F)F)cc(C(F)(F)F)c1 ; CC(=O)Oc1ccc(Br)cc1C(=O)Nc1cc(C(F)(F)F)cc(C(F)(F)F)c1]

9.7. Method 9.4 wherein the compound of Formula I is:

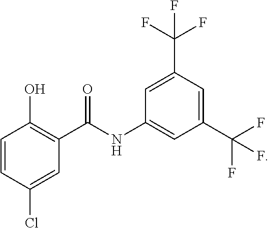 [SMILES: O=C(Nc1cc(C(F)(F)F)cc(C(F)(F)F)c1)c1cc(Cl)ccc1O]

9.8. Method 9.3 wherein R₁, R₃ and R₅ are each chloro, and R₂, R₄ and R₆ are each H.
9.9. Method 9.3 wherein R₁, R₃ and R₅ are each trifluoromethyl, and R₂, R₄ and R₆ are each H.
9.10. Any of Method 9, 10, 11, or 12 or 9.3 wherein R₆ is C₁₋₄ acyl (e.g. acetyl).
9.11. Any of Method 9, 10, 11, or 12 or 9.3 wherein R₆ is the residue of an amino acid.
9.12. Any of Method 9, 10, 11 or 12 or 9.3 wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom, e.g., wherein said 5 to 6-membered non-aromatic heterocyclic ring is selected from 1-pyrrolidinyl group, piperidino group, morpholino group, and 1-piperazinyl group, and said heterocyclic ring may be substituted with one or more substituents, e.g., independently selected from an alkyl group, an alkyl-oxy-carbonyl group, and a carboxy group; for example wherein R₆ is (morpholin-4-yl)carbonyl.
9.13. Any of Method 9, 10, 11, or 12 or 9.3 wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.
9.14. Any of Method 9, 10, 11, or 12 or 9.3 wherein R₆ is a (morpholin-4-yl)carbonyl group.
9.15. Any of Method 9, 10, 11, or 12 or 9.3 wherein R₆ is a phosphono (—PO₃), which may be substituted, e.g. dibenzylphosphono (—P(═O)(OCH₂C₆H₅)₂), or unsubstituted (—P(═O)(OH)₂).
9.16. Method 9.15 wherein R₆ is ,,P(═O)(OH)₂.
9.17. Method 9.16 wherein the prodrug of Formula I is 2-((3,5-bis(trifluoromethyl)phenyl)carbamoyl)-4-chlorophenyl dihydrogen phosphate:

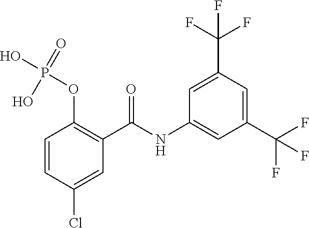 [SMILES: O=C(Nc1cc(C(F)(F)F)cc(C(F)(F)F)c1)c1cc(Cl)ccc1OP(=O)(O)O]

or a pharmaceutically acceptable salt thereof.
9.18. Method 9.17 comprising administering before, during, and/or after surgery, a pharmaceutically acceptable solution comprising a pharmaceutically acceptable salt of 2-((3,5-bis(trifluoromethyl)phenyl)carbamoyl)-4-chlorophenyl dihydrogen phosphate dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
9.19. Method 9.15 wherein the prodrug of Formula I is:

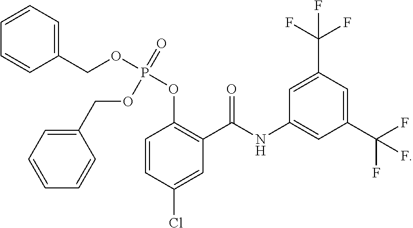 [SMILES: O=C(Nc1cc(C(F)(F)F)cc(C(F)(F)F)c1)c1cc(Cl)ccc1OP(=O)(OCc1ccccc1)OCc1ccccc1]

9.20. Method 9.15 wherein the pharmaceutically acceptable salt prodrug of Formula I is a compound of Formula Ia:

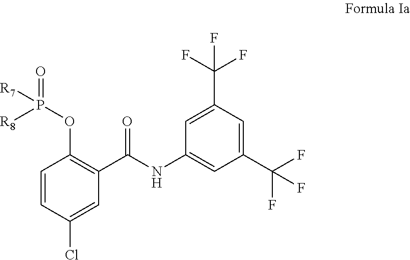

wherein one of R₇ and R₈ is OH and the other is O⁻Q⁺ or both R₇ and R₈ are O⁻Q⁺ wherein each Q⁺ is independently a pharmaceutically acceptable cation.
9.21. Method 9.20 wherein one of R₇ and R₈ is OH and the other is O⁻Q⁺.
9.22. Method 9.20 wherein both R₇ and R₈ are O⁻Q⁺.
9.23. Any of Method 9.20-9.22 wherein each Q⁺ is independently Na⁺ or K⁺.
9.24. Method 9.23 wherein each Q⁺ is N⁺.
9.25. Method 9.24 wherein the pharmaceutically acceptable salt prodrug of Formula Ia is:

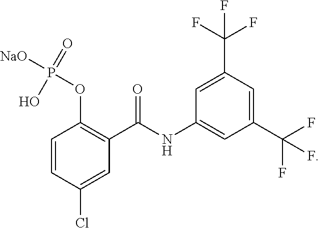

9.26. Method 9.24 wherein the pharmaceutically acceptable salt prodrug of Formula Ia is:

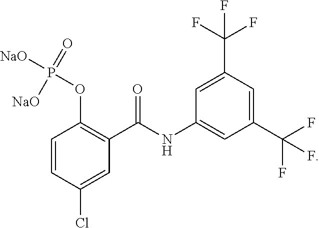

9.27. Any of Method 9.20-9.22 wherein each Q⁺ is independently an optionally substituted ammonium or iminium, e.g., protonated morpholine, mono- or di-protonated piperazine, protonated benethamine, mono- or di-protonated benzathine, trimethylglycine, mono or di-protonated chloroprocaine, mono or di-protonated hydrabamine, a mono or di-protonated amino acid (e.g., a mono- or di-protonated arginine or a mono- or di-protonated lysine), or a protonated mono- and/or poly-hydroxyalkylamine, e.g., (HO)ₙR₉NH₃⁺, [(HO)ₙR₉]₂NH₂⁺, or [(HO)ₙR₉]₃NH⁺, wherein each R₉ is independently C₁₋₈-alkyl (e.g., C₁₋₆-alkyl, e.g., C₁₋₄-alkyl, e.g., —CH₂CH₃, e.g., —CH₃) and n is 0 or each R₉ is independently C₁₋₈-alkylene (e.g., C₁₋₆-alkylene, e.g., C₁₋₄-alkylene, e.g., ,,CH₂,,CH₂,,, e.g., ,,C(CH₂)₃,,, e.g., one R₉ is ,,CH₃ and another R₉ is ,,(CH₂)₆,,) and each n is independently 1-8 (e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.
9.28. Method 9.27 wherein each Q⁺ is protonated tris(hydroxymethyl)aminomethane.
9.29. Any of Method 9.20-9.28 comprising administering before, during, and/or after surgery, a pharmaceutically acceptable solution comprising Formula Ia dissolved therein, e.g., wherein the pharmaceutically acceptable solution comprises sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride.
9.30. Method 9.18 or 9.29 wherein the concentration of

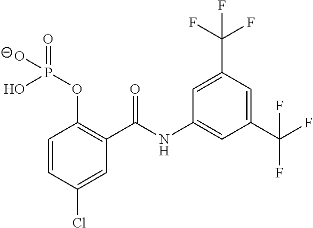 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
9.31. Method 9.18 or 9.29 wherein the concentration of

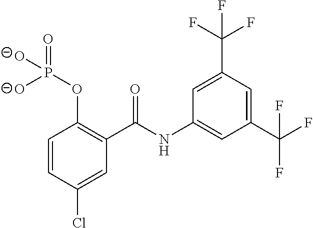 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
9.32. Any of Method 9, 10, 11, or 12 wherein the compound of Formula I is:

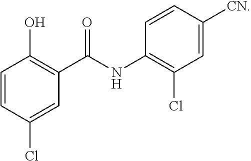 [SMILES: N#Cc1ccc(NC(=O)c2cc(Cl)ccc2O)c(Cl)c1]

9.33. Any of Method 9 wherein the phenylbenzamide is:

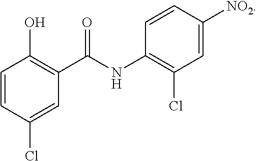 [SMILES: O=C(Nc1ccc([N+](=O)[O-])cc1Cl)c1cc(Cl)ccc1O]

9.34. Any of Method 9, 10, 11, or 12 or 9.1-9.33 comprising administering before, during, and/or after surgery, 0.1 or 0.25 mg to 2.0 g of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering before, during, and/or after surgery, the pharmaceutically acceptable salt, prodrug, or pharmaceutically acceptable salt prodrug thereof in an amount sufficient to provide 0.1 or 0.25 mg to 2.0 g of the phenylbenzamide, e.g., the compound of Formula I, e.g., N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
9.35. Any of Method 9, 10, 11, or 12 or 9.1-9.34 comprising administering before, during, and/or after surgery, 0.1 or 0.25 mg to 2.0 g of N-[3,5-bis(trifluoromethyl)phenyl]-5-chloro-2-hydroxybenzamide or a pharmaceutically acceptable salt, prodrug, or pharmaceutically acceptable salt prodrug thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering before, during, and/or after surgery, the pharmaceutically acceptable salt, prodrug, or pharmaceutically acceptable salt prodrug in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
9.36. Any of Method 9, 10, 11, or 12 or 9.1-9.35 comprising administering before, during, and/or after surgery, 0.1 or 0.25 mg to 2.0 g of

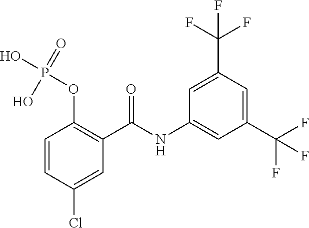 [SMILES: O=C(Nc1cc(C(F)(F)F)cc(C(F)(F)F)c1)c1cc(Cl)ccc1OP(=O)(O)O]

or a pharmaceutically acceptable salt thereof, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering before, during, and/or after surgery,

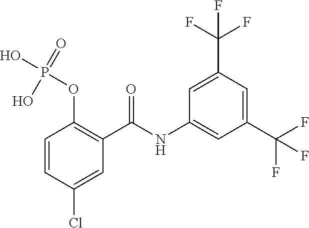 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
9.37. Any of Method 9, 10, 11, or 12 or 9.1-9.36 comprising administering before, during, and/or after surgery, a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising

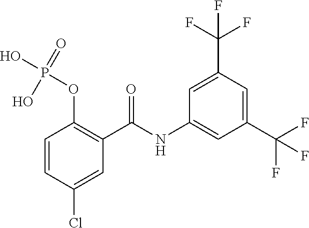 [SMILES: O=C(Nc1cc(C(F)(F)F)cc(C(F)(F)F)c1)c1cc(Cl)ccc1OP(=O)(O)O]

or a pharmaceutically acceptable salt thereof dissolved therein.
9.38. Any of Method 9, 10, 11, or 12 or 9.1-9.37 comprising administering before, during, and/or after surgery, 0.1 or 0.25 mg to 2.0 g of the compound of Formula Ia

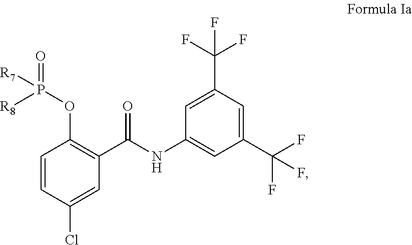

as described in any of Method 9.20-9.28, e.g., from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg, or comprising administering before, during, and/or after surgery, the compound of Formula Ia

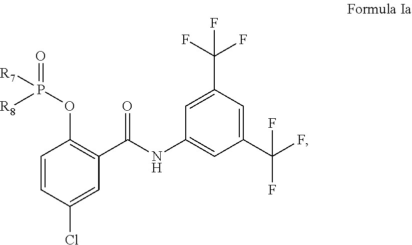

as described in any of Method 9.20-9.28, in an amount sufficient to provide 0.1 or 0.25 mg to 2.0 g of N-[3,5-bis(trifluoromethyl)phenyl]-5-chloro-2-hydroxybenzamide, e.g., in an amount sufficient to provide from 0.1 or 0.25 mg to 75 or 600 mg, e.g., from 0.1 or 0.25 or 1 or 2 or 5 or 10 or 15 or 20 mg to 50, 75, 100, 125, 150, 200, 300, 350, 400, 500, or 600 mg, or 1 g, 1.5 g, or 2.0 g, e.g., from 5 to 50, 75, 100, 125, 150, 200, 300, 350, 400, 500, or 600 mg, or 1 g, 1.5 g, or 2 g, e.g., from 5 to 500 mg, e.g., from 5 to 300 or 350 mg, e.g., from 5 to 200 mg, e.g., from 25 to 500 mg, e.g., from 25 to 300 or 350 mg, e.g., from 25 to 200 mg, e.g., from 15, 20, 30, 35, 50, or 100 to 150, 200, 300, 350, 400, 450, 500, 550, or 600 mg, e.g., from 0.5 or 1 mg to 50 mg, e.g., from 0.5 or 1 mg to 20 mg, e.g., from 0.5 or 1 mg to 10 mg, e.g., from 1 or 2 or 5 mg to 10 or 20 mg, e.g., from 1 or 2 or 3 or 4 to 5 mg, e.g., about 35 mg, e.g., about 350 mg.
9.39. Any of Method 9, 10, 11, or 12 or 9.1-9.38 comprising administering before, during, and/or after surgery, a pharmaceutically acceptable solution, e.g., sterile water for injection, a sterile solution comprising dextrose (e.g., dextrose injection 5%), a sterile solution comprising sodium chloride (e.g., 0.9% sodium chloride injection), a sterile solution comprising benzyl alcohol (e.g., bacteriostatic water for injection with benzyl alcohol or bacteriostatic sodium chloride for injection with benzyl alcohol), or Lactated Ringer's, e.g., sterile water for injection, e.g., a sterile solution comprising sodium chloride, comprising Formula Ia

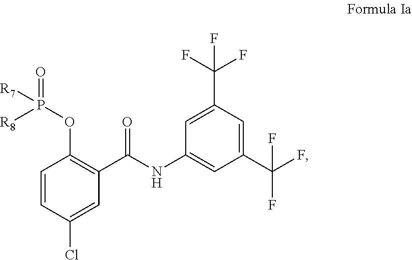

as described in any of Method 9.20-9.28, dissolved therein.
9.40. Any of Method 9.36-9.39 wherein the concentration of

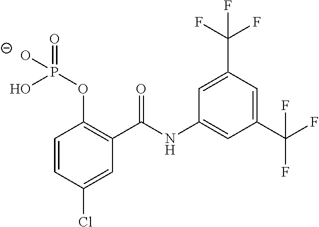 [SMILES: O=C(Nc1cc(C(F)(F)F)cc(C(F)(F)F)c1)c1cc(Cl)ccc1OP(=O)([O-])O]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
9.41. Any of Method 9.36-9.39 wherein the concentration of

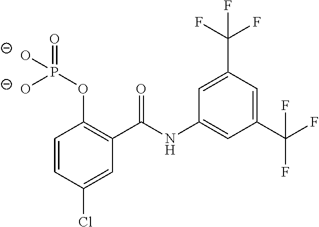 [SMILES: O=C(Nc1cc(C(F)(F)F)cc(C(F)(F)F)c1)c1cc(Cl)ccc1OP(=O)([O-])[O-]]

is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
9.42. Any of Method 9, 10, 11, or 12 or 9.1-9.41 comprising administering before, during, and/or after surgery, a dose of 0.01 or 0.1 or 0.5 mg/kg to 1 or 5 or 10 or 15 mg/kg of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
9.43. Any of Method 9, 10, 11, or 12 or 9.1-9.42 comprising administering before, during, and/or after surgery, the pharmaceutically acceptable salt, prodrug, or pharmaceutically acceptable salt prodrug of Formula I, e.g., of N-[3,5-bis(trifluoromethyl)phenyl]-5-chloro-2-hydroxybenzamide, in an amount sufficient to provide a dose of 0.01 or 0.1 or 0.5 mg/kg to 1 or 5 or 10 or 15 mg/kg of the compound of Formula I, e.g., N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
9.44. Any of Method 9, 10, 11, or 12 or 9.1-9.43 comprising administering before, during, and/or after surgery, a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide or a pharmaceutically acceptable salt, prodrug (e.g., 2-((3,5-bis(trifluoromethyl)phenyl)carbamoyl)-4-chlorophenyl dihydrogen phosphate), or pharmaceutically acceptable salt prodrug thereof (e.g., Formula Ia as described in any of Method 1.23-1.31), e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
9.45. Any of Method 9, 10, 11, or 12 or 9.1-9.44 comprising administering before, during, and/or after surgery, the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 9.20-9.28, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
9.46. Any of Method 9, 10, 11, or 12 or 9.1-9.45 comprising administering before, during, and/or after surgery, the pharmaceutically acceptable salt, prodrug, or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 9.20-9.28, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
9.47. Any of Method 9, 10, 11, or 12 or 9.1-9.46 comprising administering before, during, and/or after surgery,

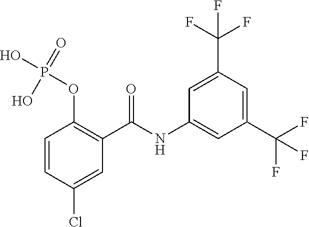 [SMILES: O=C(Nc1cc(C(F)(F)F)cc(C(F)(F)F)c1)c1cc(Cl)ccc1OP(=O)(O)O]

or a pharmaceutically acceptable salt thereof in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
9.48. Any of Method 9, 10, 11, or 12 or 9.1-9.47 comprising administering before, during, and/or after surgery,

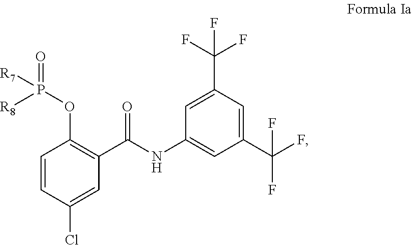

as described in any of Method 9.20-9.28, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide, e.g., a dose of 0.05 to 1 or 5 mg/kg, e.g., a dose of 0.05 to 0.1, 0.2, 0.3, 0.4, 0.5, 1, 5, 10 or 20 mg/kg, e.g., a dose of 0.5 to 1, 2, 3, 4, 5 or 10 or 20 mg/kg, e.g., a dose of 1 to 2, 3, 4, 5, 10, 20 or 50 mg/kg.
9.49. Any of Method 9, 10, 11, or 12 or 9.1-9.48 wherein the aquaporin is AQP4.
9.50. Any of Method 9, 10, 11, or 12 or 9.1-9.49 wherein the aquaporin is AQP2.
9.51. Any of Method 9, 10, 11, or 12 or 9.1-9.50 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered orally, e.g., tablet, capsule, solution, suspension, or the like.
9.52. Method 9.51 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 9.17, 9.18, or 9.20-9.31, is administered orally.
9.53. Any of Method 9, 10, 11, or 12 or 9.1-9.51 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered parenterally.
9.54. Method 9.53 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 9.17, 9.18, or 9.20-9.31, is administered parenterally.
9.55. Method 9.53 or 9.54 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
9.56. Any of Method 9.53-9.55 wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 9.17, 9.18, or 9.20-9.31, is administered by injection, e.g., subcutaneously, intramuscularly, intravenously, or intrathecally, e.g., intramuscularly or intravenously, e.g., a bolus injected subcutaneously, intramuscularly, intravenously, or intrathecally.
9.57. Any of Method 9.53-9.56 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intravenously, e.g., IV bolus and/or IV infusion, e.g., IV bolus followed by IV infusion.
9.58. Any of Method 9.53-9.57 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 9.17, 9.18, or 9.20-9.31, is administered intravenously, e.g., IV bolus and/or IV infusion, e.g., IV bolus followed by IV infusion.
9.59. Any of Method 9.53-9.56 wherein the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, is administered intramuscularly, e.g., IM bolus and/or IM infusion, e.g., IM bolus followed by IM infusion.
9.60. Any of Method 9.53-9.56 or 9.59 wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 9.17, 9.18, or 9.20-9.31, is administered intramuscularly, e.g., IM bolus and/or IM infusion, e.g., IM bolus followed by IM infusion.
9.61. Any of Method 9.53-9.60 wherein the infusion, e.g., IV or IM, is administered over 10 or 30 minutes to 72 hours, e.g., 30 minutes to 24 hours, e.g., 30 minutes to 12 hours, e.g., 30 minutes to 8 hours, e.g., 30 minutes to 6 hours, e.g., 30 minutes to 4 hours, e.g., 30 minutes to 2 hours, e.g., 30 minutes to 1 hour.
9.62. Any of Method 9, 10, 11, or 12 or 9.1-9.61 wherein the patient is human.
9.63. Any of Method 9, 10, 11, or 12 or 9.1-9.62 wherein the onset of action of any of the compounds identified in any of Methods 9, 9.3-9.17, or 9.19-9.28, is fairly rapid.
9.64. Any of Method 9, 10, 11, or 12 or 9.1-9.63 comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 9.17, 9.18, or 9.20-9.31, prior to the surgery, e.g., 12 hours or less, e.g. 8 hours or less, e.g., 6 hours or less, e.g., 3 hours or less, e.g., 2 hours or less, e.g., 1 hour or less, e.g., 30 minutes or less, e.g., 10 or 5 minutes or less, prior to surgery.
9.65. Any of Method 9, 10, 11, or 12 or 9.1-9.64 comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 9.17, 9.18, or 9.20-9.31, during surgery.
9.66. Any of Method 9, 10, 11, or 12 or 9.1-9.64 comprising administering the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 9.17, 9.18, or 9.20-9.31, after the surger.
9.67. Method 9.66 wherein the inhibitor of AQP2 or AQP4, e.g., the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 9.17, 9.18, or 9.20-9.31, e.g., comprising administering a pharmaceutically acceptable solution prepared by dissolving 2-((3,5-bis(trifluoromethyl)phenyl)carbamoyl)-4-chlorophenyl dihydrogen phosphate and tris(hydroxymethyl)aminomethane, is administered for 6 months or less after the surgery, e.g., 5 months or less, e.g., 4 months or less, e.g., 3 months or less, e.g., 2 months or less, e.g., 1 month or less, e.g., 3 weeks or less, e.g., 2 weeks or less, e.g., 1 week or less.
9.68. Any of Method 9, 10, 11, or 12 or 9.1-9.67 further comprising cooling of the heart, e.g., with a perfusion in a bath, e.g., cooling to 37° C. or less, e.g., 35° C. or less, e.g., 33° C. or less, e.g., 32° C. or less, e.g., 30° C. or less.
9.69. Any of Method 9, 10, 11, or 12 or 9.1-9.68 comprising dissolving the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof, in an aqueous solution, which solution is administered to the patient.
9.70. Method 9.69 wherein the solution further comprises an osmotic active agent (e.g., lactogionate, raffinose, citrate, gluconate), an electrolyte (Na⁺, K⁺, Ca²⁺, Mg²⁺), an H⁻ ion buffer (phosphate, histidine, N-(2-hydroxyethyl)-piperazine-N′-2-ethanesulfonic acid (HEPES) buffer), a colloid (e.g., albumin, hydroxyethyl starch), a metabolic inhibitor (e.g., allopurinol, antiprotease, chlorpromazine), a metabolite (e.g., adenosine, glutathione), or an antioxidant (e.g., amino steroid, vitamin E, deferoxamine (Desferal), or a combination thereof.
9.71. Method 9.70 wherein the solution further comprises mannitol.
9.72. Any of Method 9, 10, 11, or 12 or 9.1-9.71 wherein the concentration of the phenylbenzamide, e.g., the compound of Formula I, e.g., N-[3,5-bis(trifluoromethyl)phenyl]-5-chloro-2-hydroxybenzamide, or a pharmaceutically acceptable salt, prodrug, or pharmaceutically acceptable salt prodrug thereof is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
9.73. Any of Method 9, 10, 11, or 12 or 9.1-9.72 wherein the concentration of N-[3,5-bis(trifluoromethyl)phenyl]-5-chloro-2-hydroxybenzamide is 0.01 or 0.02 or 0.05 or 0.1 or 0.5 or 1 or 2 to 250 mM, e.g., from 0.01 or 0.1 or 0.5 to 1, 2, 5, 10, 15, 20, 25, 40, 50, 60, 75, 100, 125, 150, 175, 200, 250 mM, or 1000 mM, e.g. from 1 to 2, 5, 10, 15, 20, 25, 40, 50 or 60 mM, e.g., from 5, 10, 15, 20, 25, or 50 to 100, 200, 250, 300, 400, 500, or 1000 mM, e.g., about 2, 20, or 200 mM, e.g., about 5, 10, 50, 500, 500, or 1000 mM.
9.74. Method 9, 10, 11, or 12 or 9.1-9.73 wherein the solution is administered to the patient (e.g., as a bolus or continuous infusion) before surgery begins, or both before and during surgery, or before, during and after surgery.
9.75. Method 9, 10, 11, or 12 or 9.1-9.74 wherein the heart is bathed in the solution during the surgery.
9.76. Method 9, 10, 11, or 12 or 9.1-9.75 wherein the heart is perfused with the solution during the surgery.

“Before, during, and/or after surgery” includes each separately and in any combination thereof. For example, the phenylbenzamide may be administered before surgery or during surgery or after surgery. In addition, for example, the phenylbenzamide may be administered before and during surgery or before, during, and after surgery, or during and after surgery.

Further provided is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, for use in the protection of the heart during heart surgery, e.g., open heart surgery, e.g., for use in any of Methods 9, 9.1, et seq.

Further provided is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in the manufacture of a medicament for use in the protection of the heart during heart surgery, e.g., open heart surgery, e.g., for use in any of Methods 9, 9.1, et seq.

Further provided is a pharmaceutical composition comprising a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in combination with a pharmaceutically acceptable diluent or carrier for use in the protection of the heart during heart surgery, e.g., open heart surgery, e.g., for use in any of Methods 9, 9.1, et seq.

Further provided is an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, for use in the protection of the heart during heart surgery, e.g., open heart surgery, e.g., for use in any of Methods 10, 9.1, et seq.

Further provided is an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in the manufacture of a medicament for the protection of the heart during heart surgery, e.g., open heart surgery, e.g., for use in any of Methods 10, 9.1, et seq.

Further provided is a pharmaceutical composition comprising an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in combination with a pharmaceutically acceptable diluent or carrier for use in the protection of the heart during heart surgery, e.g., open heart surgery, e.g., for use in any of Methods 10, 9.1, et seq.

Further provided is use of a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in an amount effective to inhibit an aquaporin for the protection of the heart during heart surgery, e.g., open heart surgery, e.g., for use in any of Methods 11, 9.1, et seq.

Further provided is use of a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in an amount effective to inhibit an aquaporin in the manufacture of a medicament for the protection of the heart during heart surgery, e.g., open heart surgery, e.g., for use in any of Methods 11, 9.1, et seq.

Further provided is a pharmaceutical composition comprising a phenylbenzamide, e.g., a compound of Formula I or a pharmaceutically acceptable salt, prodrug, or pharmaceutically acceptable salt prodrug thereof, e.g., Formula Ia, N-[3,5-bis(trifluoromethyl)phenyl]-5-chloro-2-hydroxybenzamide, or 2-((3,5-bis(trifluoromethyl)phenyl)carbamoyl)-4-chlorophenyl dihydrogen phosphate, as hereinbefore described, in an amount effective to inhibit an aquaporin in combination with a pharmaceutically acceptable diluent or carrier for use in the protection of the heart during heart surgery, e.g., open heart surgery, e.g., for use in any of Methods 11, 9.1, et seq.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Example 1: Cardiac Allograft Rejection Model

The ability of an APQ inhibitor of the present disclosure to protect against transplant rejection is determined in an intraabdominal heterotopic vascularized cardiac transplantation model between genetically mismatched mice. Four conditions are compared: the use of an APQ4 inhibitor alone, the use of a CTLA4 Ig alone, the combination of an APQ4 inhibitor with a CTLA4 Ig, and a control.

APQ4 Inhibitor Protocol

The donor mouse (BalbC, fully genetically mismatched with respect to the recipient mouse which is B6,, C57BL/6) is given a 10 mg/kg IP bolus of 2-((3,5-bis(trifluoromethyl)phenyl)carbamoyl)-4-chlorophenyl dihydrogen phosphate in TrisBase 30 minutes prior to surgery.

Before removing the heart, the donor is perfused with Ringer's solution containing 10 μM N-[3,5-bis(trifluoromethyl)phenyl]-5-chloro-2-hydroxybenzamide. After removal from the donor, the heart is stored at 0-4° C. for 8 hours in Ringer's solution containing 10 μM N-[3,5-bis(trifluoromethyl)phenyl]-5-chloro-2-hydroxybenzamide (Cold Ischemic Storage).

30 Minutes prior to surgery, the recipient mouse is given a 10 mg/kg IP bolus of 2-((3,5-bis(trifluoromethyl)phenyl)carbamoyl)-4-chlorophenyl dihydrogen phosphate in TrisBase. The heart is then transplanted into the recipient mouse (B6 mouse) using the vascularized heterotopic cardiac transplantation model. After closing, the recipient mouse receives 10 mg/kg IP injection of 2-((3,5-bis(trifluoromethyl)phenyl)carbamoyl)-4-chlorophenyl dihydrogen phosphate in TrisBase every 6 hours for 5 days.

CTLA4 Ig Protocol

The recipient mouse (B6) is administered a single dose of Belatacept IP at 10 mg/kg on the day of the transplant surgery as well as a single dose of Belatacept IP at 10 mg/kg 24 hours after surgery.

As a negative control, both the donor and recipient mice receive IP administration of TrisBase with neither an APQ4 inhibitor nor a CTLA4 Ig. Results

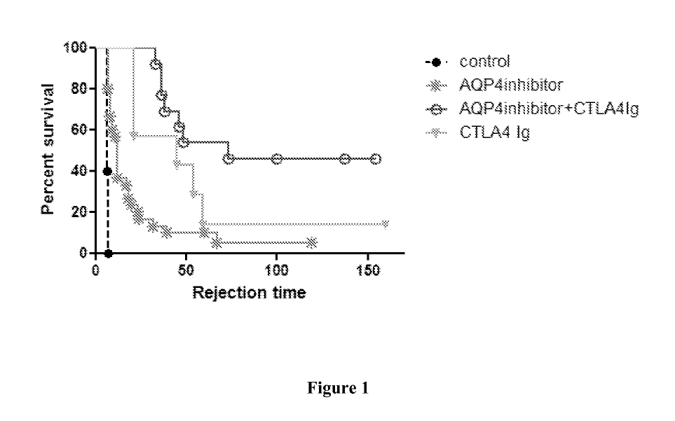

The recipient mice are monitored until their hearts stops beating. The results (FIG. 1) demonstrate that the blockade of APQ4 in addition to CTLA4 Ig treatment results in significantly improved survival. The combination displays a synergistic effect that exceeds the benefit obtained from either treatment regimen alone. For the control mice, all mice are dead within 7 days of surgery. Treatment with CTLA4 Ig alone results in approximately 60% survival after 20 days, about 40% survival after 50 days, and about 13% survival from about 60 days through the end of monitoring at 150 days. Treatment with the APQ4 inhibitor alone results in about 30% survival after 20 days, about 10% survival after 50 days, and about 5% survival from about 60 days through the end of monitoring at 120 days. The combination of the APQ4 protocol with the CTLA4 Ig protocol results in 100% survival through about 30 days, about 60% survival after 50 days, and about 45% survival from about 70 days through the end of monitoring at 150 days.

### Example 2: Cardiac Explant Experiment

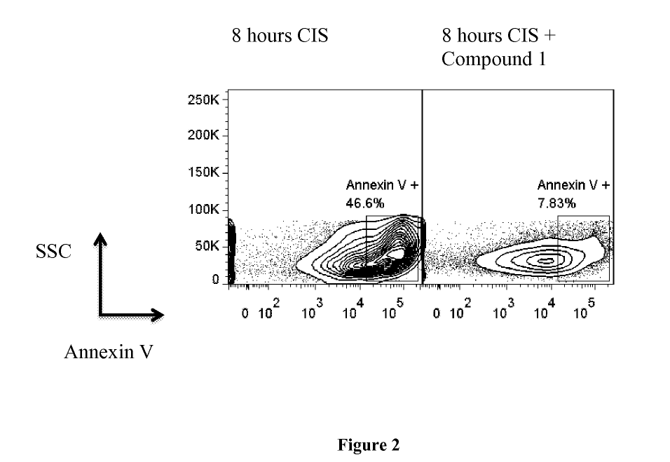

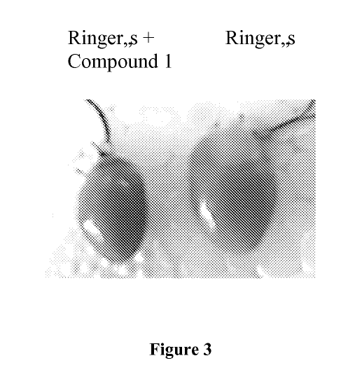

This experiment is a model for determining the extent of apoptosis occurring in an explanted heart from BALB/c mice. Hearts are perfused with Ringer's solution either supplemented with 10 μM of N-[3,5-bis(trifluoromethyl)phenyl]-5-chloro-2-hydroxybenzamide (Compound 1), or without such supplementation. The explanted hearts are then placed in Cold Ischemic Storage (at 0-4° C.) in their respective buffer solutions for 8 hours. The hearts are then photographed and triturated for analysis of apoptosis using Annexin V flow cytometry. Annexin V is used to label phosphatidylserine on the surface of cells, and this is a conventional marker for cells that have undergone apoptosis. It is found that hearts maintained in Ringer's solution for 8 hours in cold storage show about 46% apoptotic cells. In contrast, hearts maintained in the APQ4-inhibitor supplemented Ringer's solution show only about 8% apoptotic cells. See FIG. 2. In addition, FIG. 3 shows that a heart maintained in un-supplemented Ringer's solution shows considerable swelling compared to a heart maintained in supplemented solution.

1. A method for treatment or prophylaxis of transplant rejection, inhibiting rejection of transplanted biological material, or prophylaxis, treatment, or control of edema consequent to a transplant, comprising administering to a patient in need thereof an effective amount of a phenylbenzamide, e.g., an effective amount of a compound of Formula I: 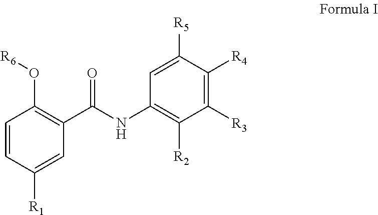
wherein R₁, R₂, R₃, R₄, and R₅ are selected from H, halogen, halogenated C₁₋₄ alkyl (e.g., trifluoromethyl), and cyano; and
R₆ is H;
or a pharmaceutically acceptable salt, prodrug {e.g., wherein R₆ is a physiologically hydrolyzable and acceptable acyl (e.g., acetyl) or a physiologically hydrolyzable and acceptable phosphono (—PO₃), which may be substituted, e.g. dibenzylphosphono (—P(═O)(OCH₂C₆H₅)₂), or unsubstituted (—P(═O)(OH)₂)}, or a pharmaceutically acceptable salt prodrug (e.g., —PO₃²⁻Q⁺Q⁺ or —PO₃²⁻Q²⁺, wherein Q is a pharmaceutically acceptable cation) thereof.

2. A method for treatment or prophylaxis of transplant rejection, inhibiting rejection of transplanted biological material, or prophylaxis, treatment, or control of edema consequent to a transplant, comprising administering to a patient in need thereof an effective amount of an aquaporin inhibitor, e.g., a compound binding to an aquaporin, e.g. AQP4, e.g., an inhibitor of AQP2 or AQP4, e.g., AQP4, wherein the aquaporin inhibitor is a phenylbenzamide, e.g., a compound of Formula I as recited in claim 1.

3. A method for treatment or prophylaxis of transplant rejection, inhibiting rejection of transplanted biological material, or prophylaxis, treatment, or control of edema consequent to a transplant, comprising administering to a patient in need thereof an aquaporin inhibitor, e.g., a compound binding to an aquaporin, e.g. AQP4, e.g., an inhibitor of AQP2 or AQP4, e.g., AQP4, in an amount effective to inhibit the aquaporin, e.g., AQP4, wherein the aquaporin inhibitor is a phenylbenzamide, e.g., a compound of Formula I, as recited in claim 1.

4. A method to inhibit an aquaporin in a patient suffering from transplant rejection, to inhibit an aquaporin to inhibit rejection of transplanted biological material, or to inhibit an aquaporin for prophylaxis, treatment, or control of edema consequent to a transplant, comprising administering to a patient in need thereof an aquaporin inhibitor, e.g., a compound binding to an aquaporin, e.g. AQP4, e.g., an inhibitor of AQP2 or AQP4, e.g., AQP4, in an amount effective to inhibit the aquaporin, e.g., AQP4, wherein the aquaporin inhibitor is a phenylbenzamide, e.g., a compound of Formula I, as recited in claim 1.

5. A method for treatment of a cell, tissue, or organ donor comprising administering to the donor, before and/or after removal of the cell, tissue, or organ, an effective amount of a phenylbenzamide, e.g., an effective amount of a compound of Formula I, as recited in claim 1.

6. A method of preservation of biological material, e.g., cell, tissue, or organ preservation, comprising contacting the biological material with a phenylbenzamide, e.g., a compound of Formula I, as recited in claim 1.

7. A method of preservation of biological material, e.g., cell, tissue, or organ preservation, comprising contacting the biological material with an effective amount of an aquaporin inhibitor, e.g., a compound binding to an aquaporin, e.g., AQP4, e.g., an inhibitor of AQP2 or AQP4, e.g., AQP4, wherein the aquaporin inhibitor is a phenylbenzamide, e.g., a compound of Formula I, as recited in claim 1.

8. A method of preservation of biological material, e.g., cell, tissue, or organ preservation, comprising contacting the biological material with an aquaporin inhibitor, e.g., a compound binding to an aquaporin, e.g., AQP4, e.g., an inhibitor of AQP2 or AQP4, e.g., AQP4, in an amount effective to inhibit the aquaporin, wherein the aquaporin inhibitor is a phenylbenzamide, e.g., a compound of Formula I, as recited in claim 1.

9. A method to inhibit an aquaporin to preserve biological material, e.g., cell, tissue, or organ preservation, comprising contacting the biological material with an effective amount of an aquaporin inhibitor, e.g., a compound binding to an aquaporin, e.g., AQP4, e.g., an inhibitor of AQP2 or AQP4, e.g., AQP4, in an amount effective to inhibit the aquaporin, wherein the aquaporin inhibitor is a phenylbenzamide, e.g., a compound of Formula I, as recited in claim 1.

10. A method of protecting a heart during heart surgery, comprising contacting the heart with a phenylbenzamide, e.g., a compound of Formula I, as recited in claim 1.

11. The method of any one of claims 1-4 comprising treatment or prophylaxis of transplant rejection.

12. The method of any one of claims 1-4 comprising rejection of transplanted biological material.

13. The method of any one of claims 1-4 comprising prophylaxis, treatment, or control of edema consequent to a transplant.

14. The method of any one of claims 1-13, wherein the phenylbenzamide is as described in U.S. Patent Publication No. 2010/0274051.

15. The method of any one of claims 1-13, wherein the phenylbenzamide is as described in in U.S. Pat. Nos. 7,626,042 or 7,700,655.

16. The method of any one of claims 1-13, wherein R₁ is selected from trifluoromethyl, chloro, fluoro, and bromo; R₃ and R₅ are the same or different and selected from trifluoromethyl, chloro, fluoro, and bromo; and R₂ and R₄ are both H.

17. The method of claim 16, wherein R₁ is selected from chloro and bromo; R₃ and R₅ are both trifluoromethyl; and R₂, R₄ and R₆ are all H, e.g., wherein the compound of Formula I is selected from:

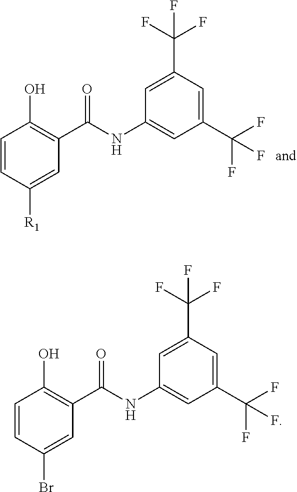

18. The method of any one of claims 1-13, wherein R₆ is acetyl.

19. The method of any one of claims 1-13, wherein R₁ is selected from chloro and bromo; R₃ and R₅ are both trifluoromethyl; and R₂ and R₄ are H and R₆ is acetyl, e.g., wherein the compound of Formula I is selected from:

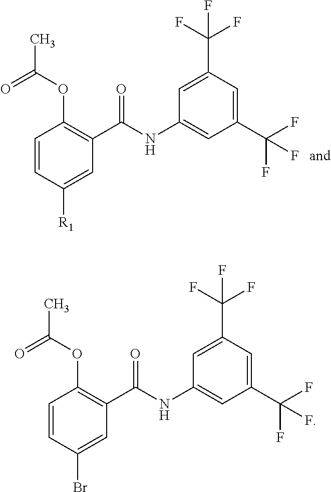

20. The method of claim 17, wherein the compound of Formula I is:

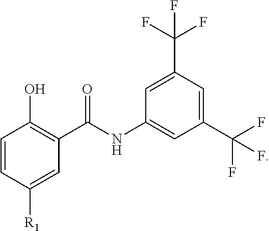

21. The method of any one of claims 1-13, wherein R₁, R₃ and R₅ are each chloro, and R₂, R₄ and R₆ are each H.

22. The method of any one of claims 1-13, wherein R₁, R₃ and R₅ are each trifluoromethyl, and R₂, R₄ and R₆ are each H.

23. The method of any one of claims 1-13, wherein R₆ is C₁₋₄ acyl (e.g. acetyl).

24. The method of any one of claims 1-13, wherein R₆ is the residue of an amino acid.

25. The method of any one of claims 1-13, wherein R₆ is a 5 to 6-membered non-aromatic heterocyclic ring-carbonyl group, for example a 5 to 6-membered non-aromatic heterocyclic ring-carbonyl group which comprises at least one nitrogen atom as ring-constituting atoms (ring forming atoms) of said heterocyclic ring and binds to the carbonyl group at the nitrogen atom.

26. The method of any one of claims 1-13, wherein R₆ is a N,N-di-substituted carbamoyl group, wherein two substituents of said carbamoyl group may combine to each other, together with the nitrogen atom to which they bind, to form a nitrogen-containing heterocyclic group which may be substituted.

27. The method of any one of claims 1-13, wherein R₆ is a (morpholin-4-yl)carbonyl group.

28. The method of any one of claims 1-13, wherein R₆ is a phosphono (—PO₃), which may be substituted, e.g. dibenzylphosphono (—P(═O)(OCH₂C₆H₅)₂), or unsubstituted (—P(═O)(OH)₂).

29. The method of claim 28, wherein R₆ is ,,P(═O)(OH)₂.

30. The method of claim 29, wherein the prodrug of Formula I is 2-((3,5-bis(trifluoromethyl)phenyl)carbamoyl)-4-chlorophenyl dihydrogen phosphate: 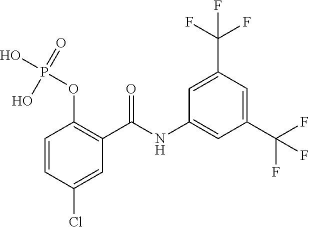 [SMILES: O=C(Nc1cc(C(F)(F)F)cc(C(F)(F)F)c1)c1cc(Cl)ccc1OP(=O)(O)O]
or a pharmaceutically acceptable salt thereof.

31. The method of claim 30 wherein the pharmaceutically acceptable salt of 2-((3,5-bis(trifluoromethyl)phenyl)carbamoyl)-4-chlorophenyl dihydrogen phosphate is dissolved in a pharmaceutically acceptable solution.

32. The method of claim 28, wherein the prodrug of Formula I is:

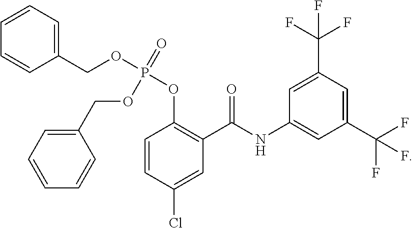 [SMILES: O=C(Nc1cc(C(F)(F)F)cc(C(F)(F)F)c1)c1cc(Cl)ccc1OP(=O)(OCc1ccccc1)OCc1ccccc1]

33. The method of claim 28 or 30, wherein the pharmaceutically acceptable salt prodrug of Formula I is a compound of Formula Ia: 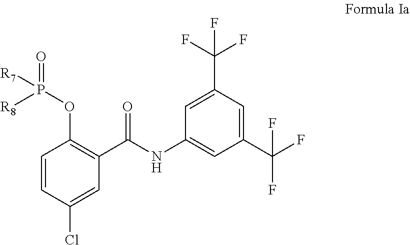
wherein one of R₇ and R₈ is OH and the other is O⁻Q⁺ or both R₇ and R₈ are O⁻Q⁺ wherein each Q⁺ is independently a pharmaceutically acceptable cation.

34. The method of claim 33, wherein one of R₇ and R₈ is OH and the other is O⁻Q⁺.

35. The method of claim 33, wherein both R₇ and R₈ are O⁻Q⁺.

36. The method of any one of claims 33-35, wherein each Q⁺ is independently Na⁺ or K⁺.

37. The method of claim 36, wherein each Q⁺ is Na⁺.

38. The method of claim 37, wherein the pharmaceutically acceptable salt prodrug of Formula Ia is:

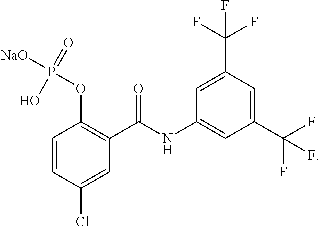

39. The method of claim 37, wherein the pharmaceutically acceptable salt prodrug of Formula Ia is:

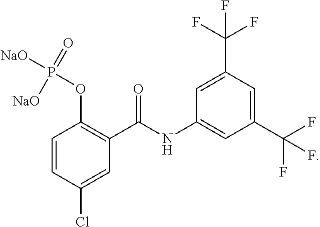

40. The method of any of claims 33-35, wherein each Q⁺ is independently an optionally substituted ammonium or iminium, e.g., a protonated mono- and/or poly-hydroxyalkylamine, e.g., H₃NR₂₀⁺, H₂NR₂₀R₂₁⁺, HNR₂₀R₂₁R₂₂⁺ wherein each R₂₀, R₂₁, and R₂₂ are independently C₁₋₈-alkyl (e.g., C₁₋₆-alkyl, e.g. C₁₋₄-alkyl, e.g., C₂-alkyl, e.g., —CH₃) optionally substituted with one or more —OH (e.g., optionally substituted with 1-8 —OH, e.g., 1, 2, 3, 4, 5, or 6), e.g., protonated tris(hydroxymethyl)aminomethane, protonated meglumine, protonated dimethylethanolamine, protonated diethylamine, protonated diethylethanolamine, and/or protonated diethanolamine), e.g., any of the preceding wherein the optionally substituted ammonium or iminium has a pKa between 6, 7, 8, 9, or 10 and 11, e.g., between 6, 7, 8, or 9 and 10, e.g., between 7 and 9, e.g., between 8 and 9.

41. The method of claim 40, wherein each Q⁺ is protonated tris(hydroxymethyl)aminomethane, e.g., the method of claim 34 wherein the compound of Formula Ia is 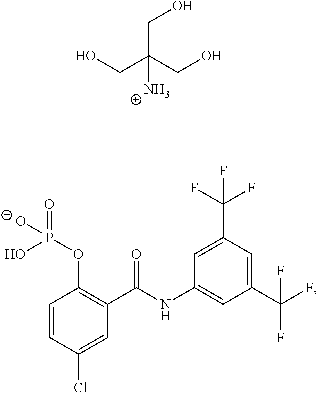 [SMILES: [NH3+]C(CO)(CO)CO ; O=C(Nc1cc(C(F)(F)F)cc(C(F)(F)F)c1)c1cc(Cl)ccc1OP(=O)([O-])O]
e.g., the method of claim 40 wherein the compound of Formula Ia is

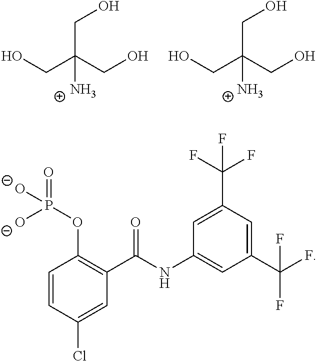 [SMILES: [NH3+]C(CO)(CO)CO ; [NH3+]C(CO)(CO)CO ; O=C(Nc1cc(C(F)(F)F)cc(C(F)(F)F)c1)c1cc(Cl)ccc1OP(=O)([O-])[O-]]

42. The method of any one of claims 33-41 wherein Formula Ia is dissolved in a pharmaceutically acceptable solution.

43. The method of claim 31 or 42, wherein the concentration of 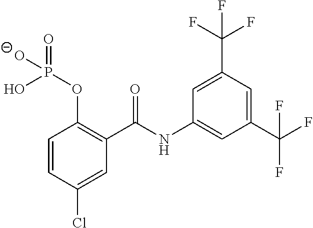 [SMILES: O=C(Nc1cc(C(F)(F)F)cc(C(F)(F)F)c1)c1cc(Cl)ccc1OP(=O)([O-])O]
is 0.01 or 0.02 or 0.05 or 0.1 or 0.5 or 1 or 2 to 250 mM.

44. The method of claim 31 or 42, wherein the concentration of 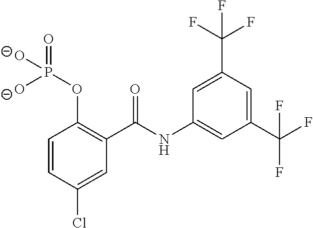 [SMILES: O=C(Nc1cc(C(F)(F)F)cc(C(F)(F)F)c1)c1cc(Cl)ccc1OP(=O)([O-])[O-]]
is 0.01 or 0.02 or 0.05 or 0.1 or 0.5 or 1 or 2 to 250 mM.

45. The method of any one of claims 1-13, wherein the compound of Formula I is:

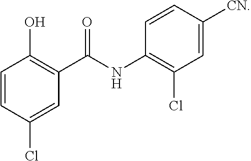 [SMILES: N#Cc1ccc(NC(=O)c2cc(Cl)ccc2O)c(Cl)c1]

46. The method of any one of claims 1-13, wherein the phenylbenzamide is:

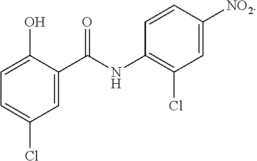 [SMILES: O=C(Nc1ccc([N+](=O)[O-])cc1Cl)c1cc(Cl)ccc1O]

47. The method of any one of claims 1-5 or 10-46 comprising administering 0.1 or 0.25 mg to 2.0 g of the compound of Formula I.

48. The method of any one of claims 1-5 or 10-47 comprising administering 0.1 or 0.25 mg to 2.0 g of N-[3,5-bis(trifluoromethyl)phenyl]-5-chloro-2-hydroxybenzamide or a pharmaceutically acceptable salt, prodrug, or pharmaceutically acceptable salt prodrug thereof.

49. The method of any one of claims 1-5 or 10-48 comprising administering 0.1 or 0.25 mg to 2.0 g of 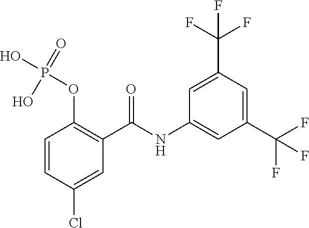 [SMILES: O=C(Nc1cc(C(F)(F)F)cc(C(F)(F)F)c1)c1cc(Cl)ccc1OP(=O)(O)O]
or a pharmaceutically acceptable salt thereof or comprising administering 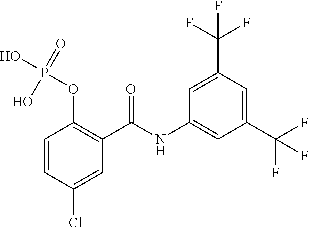 [SMILES: O=C(Nc1cc(C(F)(F)F)cc(C(F)(F)F)c1)c1cc(Cl)ccc1OP(=O)(O)O]
or a pharmaceutically acceptable salt thereof in an amount sufficient to provide 0.1 or 0.25 mg to 2.0 g of N-[3,5-bis(trifluoromethyl)phenyl]-5-chloro-2-hydroxybenzamide.

50. The method of any one of claims 1-5 or 10-49 comprising administering a pharmaceutically acceptable solution comprising 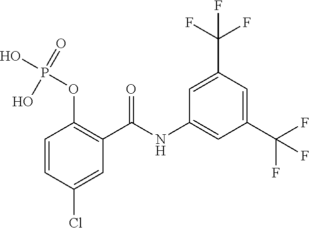 [SMILES: O=C(Nc1cc(C(F)(F)F)cc(C(F)(F)F)c1)c1cc(Cl)ccc1OP(=O)(O)O]
or a pharmaceutically acceptable salt thereof dissolved therein.

51. The method of any one claims 1-5 of 10-50 comprising administering a pharmaceutically acceptable solution comprising 0.1 or 0.25 mg to 2.0 g of the compound of Formula Ia 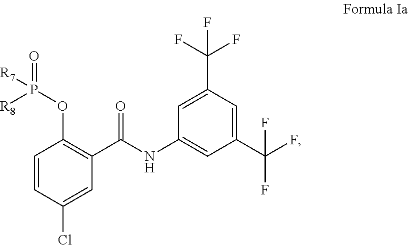
as described in any of Method 2.23-2.31 or comprising administering the compound of Formula Ia 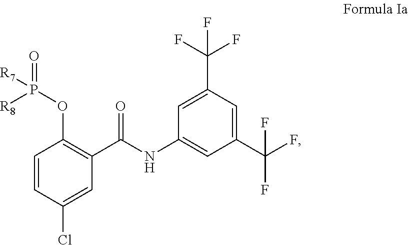
as described in any of Method 2.23-2.31, in an amount sufficient to provide 0.1 or 0.25 mg to 2.0 g of N-[3,5-bis(trifluoromethyl)phenyl]-5-chloro-2-hydroxybenzamide.

52. The method of any one of claims 1-5 of 10-51 comprising administering a pharmaceutically acceptable solution comprising Formula Ia 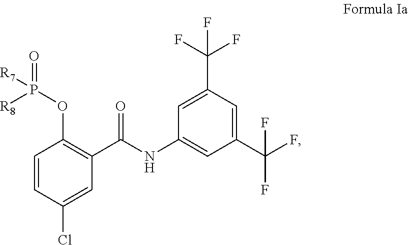
as described in any of Method 2.23-2.31, dissolved therein.

53. The method of any one of claims 49-52, wherein the concentration of 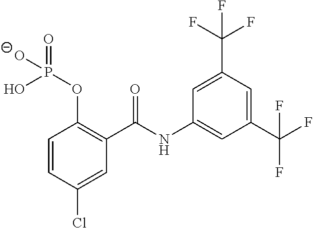 [SMILES: O=C(Nc1cc(C(F)(F)F)cc(C(F)(F)F)c1)c1cc(Cl)ccc1OP(=O)([O-])O]
is 0.01 or 0.02 or 0.05 or 0.1 or 0.5 or 1 or 2 to 250 mM.

54. The method of any one of claims 49-52, wherein the concentration of 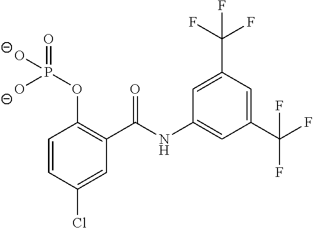 [SMILES: O=C(Nc1cc(C(F)(F)F)cc(C(F)(F)F)c1)c1cc(Cl)ccc1OP(=O)([O-])[O-]]
is 0.01 or 0.02 or 0.05 or 0.1 or 0.5 or 1 or 2 to 250 mM.

55. The method of any one of claims 1-5 of 10-54 comprising administering a dose of 0.01 or 0.1 or 0.5 mg/kg to 1 or 5 or 10 or 15 mg/kg of the aquaporin.

56. The method of any one of claims 1-5 of 10-55 comprising administering the pharmaceutically acceptable salt, prodrug, or pharmaceutically acceptable salt prodrug of Formula I, e.g., of N-[3,5-bis(trifluoromethyl)phenyl]-5-chloro-2-hydroxybenzamide, in an amount sufficient to provide a dose of 0.01 or 0.1 or 0.5 mg/kg to 1 or 5 or 10 or 15 mg/kg of the compound of Formula I.

57. The method of any one of claims 1-5 of 10-56 comprising administering a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide or a pharmaceutically acceptable salt, prodrug (e.g., 2-((3,5-bis(trifluoromethyl)phenyl)carbamoyl)-4-chlorophenyl dihydrogen phosphate), or pharmaceutically acceptable salt prodrug thereof (e.g., Formula a as described in any of Method 1.23-1.31).

58. The method of any one of claims 1-5 of 10-57 comprising administering the prodrug or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 1.23-1.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide.

59. The method of any one of claims 1-5 of 10-58 comprising administering the prodrug or pharmaceutically acceptable salt prodrug of N-[3,5-bis(trifluoromethyl)phenyl]-5-chloro-2-hydroxybenzamide, e.g., 2-((3,5-bis(trifluoromethyl)phenyl)carbamoyl)-4-chlorophenyl dihydrogen phosphate or Formula Ia as described in any of Method 1.23-1.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide.

60. The method of any one of claims 1-5 of 10-59 comprising administering 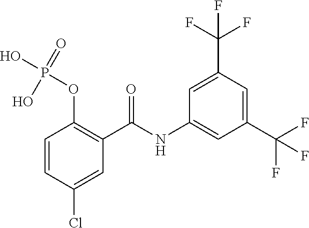 [SMILES: O=C(Nc1cc(C(F)(F)F)cc(C(F)(F)F)c1)c1cc(Cl)ccc1OP(=O)(O)O]
or a pharmaceutically acceptable salt in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide.

61. The method of any one of claims 1-5 of 10-60 comprising administering 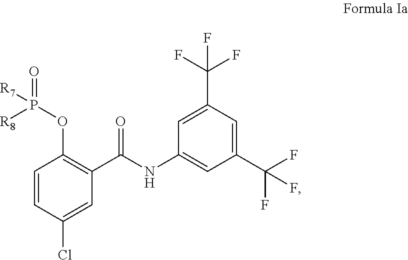
as described in any of Method 1.23-1.31, in an amount sufficient to provide a dose of 0.01 or 0.1 or 0.5 mg/kg to 1 or 5 or 10 or 15 mg/kg of N-[3,5-bis(trifluoromethyl)phenyl]-5-chloro-2-hydroxybenzamide.

62. The method of any one of claims 1-4 or 11-61, wherein the transplant rejection is consequent to an autograft.

63. The method of any one of claims 1-4 or 11-61, wherein the transplant rejection is consequent to a syngeneic graft.

64. The method of any one claims 1-4 or 11-61, wherein the transplant rejection is consequent to an isograft.

65. The method of any one of claims 1-4 or 11-61, wherein the transplant rejection is consequent to an allograft.

66. The method of any one of claims 1-4 or 11-61, wherein the transplant rejection is consequent to a xenograft.

67. The method of any one of claims 1-4 or 11-66, wherein the transplant rejection is consequent to a cell transplant, e.g., hematopoietic stem cell transplant, lymphocyte transplant, or pancreatic islet cell transplant, e.g., hematopoietic stem cell transplant, e.g., lymphocyte transplant, e.g., pancreatic islet cell transplant.

68. The method of any one of claims 1-4 or 11-66, wherein the transplant rejection is consequent to a tissue transplant.

69. The method of claim 68, wherein the tissue is bone, tendon, cartilage, connective tissue, skin, cornea, sclera, heart valve, nerve, and vessel.

70. The method of any one of claims 1-4 or 11-66, wherein the transplant rejection is consequent to transplant of an organ or a portion thereof.

71. The method of claim 70, wherein the organ is a kidney.

72. The method of claim 70, wherein the organ is the liver.

73. The method of claim 70, wherein the organ is the pancreas.

74. The method of claim 70, wherein the organ is a lung.

75. The method of claim 70, wherein the organ is the heart.

76. The method of claim 70, wherein the organ is the thymus.

77. The method of claim 70, wherein the organ is the intestine.

78. The method of claim 70, wherein the organ is the uterus.

79. The method of any one of claims 1-4 or 11-66, wherein the transplant rejection is consequent to a face, limb (e.g., hand), eye, trachea, muscle, or esophagus transplant.

80. The method of any of the preceding claims, wherein the transplant rejection is hyperacute or accelerated rejection, e.g., hyperacute rejection, e.g., accelerated rejection.

81. The method of any one of claims 1-4 or 11-66, wherein the transplant rejection is acute rejection.

82. The method of any one of claims 1-4 or 11-66, wherein the transplant rejection is chronic rejection.

83. The method of any one of the preceding claims, wherein the aquaporin is AQP4.

84. The method of any one of the preceding claims, wherein the aquaporin is AQP2.

85. The method of any one of claims 1-5 or 10-84, wherein the aquaporin inhibitor is administered orally, e.g., tablet, capsule, solution, suspension, or the like.

86. The method of claim 85, wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 1.20, 1.21, or 1.23-1.34, is administered orally.

87. The method of any one of claims 1-5 or 10-84, wherein the aquaporin inhibitor is administered parenterally.

88. The method of claim 87, wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 1.20, 1.21, or 1.23-1.34, is administered parenterally.

89. The method of claim 87 or 88, wherein the aquaporin inhibitor is administered by injection.

90. The method of any one of claims 87-89, wherein N-[3,5-bis(trifluoromethyl)phenyl]-5-chloro-2-hydroxybenzamide or a pharmaceutically acceptable salt, prodrug, or pharmaceutically acceptable salt prodrug thereof, e.g., as described in any of Method 1.20, 1.21, or 1.23-1.34, is administered by injection.

91. The method of any one of claims 87-90, wherein the aquaporin inhibitor is administered intravenously.

92. The method of any of claims 87-91, wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 1.20, 1.21, or 1.23-1.34, is administered intravenously.

93. The method of any one of claims 87-90, wherein the aquaporin inhibitor is administered intramuscularly, e.g., IM bolus and/or IM infusion, e.g., IM bolus followed by IM infusion.

94. The method of any one of claims 87-90 or 93, wherein N-[3,5-bis(trifluoromethyl)phenyl]-5-chloro-2-hydroxybenzamide, or a pharmaceutically acceptable salt, prodrug, or pharmaceutically acceptable salt prodrug thereof, e.g., as described in any of Method 1.20, 1.21, or 1.23-1.34, is administered intramuscularly.

95. The method of any one of claims 87-94, wherein the infusion, e.g., IV or IM, is administered over 10 or 30 minutes to 72 hours.

96. The method of any one of claims 1-5 or 11-95 comprising concurrently or sequentially administering another treatment for transplant rejection.

97. The method of any one claims 1-5 or 11-96 comprising concurrently or sequentially administering an immunosuppressant (e.g., a corticosteroid (e.g., prednisone, prednisolone, methylprednisolone, hydrocortisone, dexamethasone), a calcineurin inhibitor (e.g., cyclosporine, tacrolimus), a purine metabolism inhibitor (e.g., azathioprine, mycophenolate mofetil), a rapamycin (e.g., sirolimus, everolimus), an immunosuppressive Ig (e.g., antilymphocyte globulin, antithymocyte globulin, anti-Tac antibody), a monoclonal antibody (mAb) (e.g., OKT3, an anti-IL-2 receptor monoclonal antibody (e.g., basiliximab, daclizumab)), or an agent that inhibits T-cell costimulatory pathways (e.g., a cytotoxic T lymphocyte-associated antigen 4 (CTLA-4)-IgG1 fusion protein, belatacept), or a combination thereof.

98. The method of any one of claims 1-5 or 11-97 further comprising induction of chimerism using nonmyeloablative pretransplantation treatment (e.g., with cyclophosphamide, thymic irradiation, antithymocte globulin, or cyclosporin, or a combination thereof).

99. The method of any one claims 1-5 or 11-98 further comprising total body irradiation.

100. The method of any one of claims 1-5 or 10-99, wherein the patient is human.

101. The method of any one of the preceding claims, wherein the onset of action of any of the compounds identified in any of Methods 1, 1.6-1.20, or 1.11-1.31, Methods 2, 2.6-1.20, or 2.22-2.31, Methods 3, 3.6-3.20, or 3.22-3.31, Methods 4, 4.6-4.20, or 4.22-4.31, is fairly rapid.

102. The method of any one of claims 1-4 or 11-101 comprising administering the aquaporin inhibitor prior to transplantation.

103. The method of any one of claims 1-4 or 11-102 comprising administering the aquaporin inhibitor contemporaneously with transplantation.

104. The method of any one of claims 1-4 or 11-103 comprising administering the aquaporin inhibitor after transplantation.

105. The method of claim 104, wherein the aquaporin inhibitor is administered for 6 months or less after the transplant, e.g., 5 months or less, e.g., 4 months or less, e.g., 3 months or less, e.g., 2 months or less, e.g., 1 month or less, e.g., 3 weeks or less, e.g., 2 weeks or less, e.g., 1 week or less.
